(19) United States
(12) Patent Application Publication
(10) Pub. No.: US 2017/0080102 A1
(43) Pub. Date: Mar. 23, 2017

(54) METHODS FOR CHARACTERIZING AND TREATING ACUTE MYELOID LEUKEMIA

(71) Applicant: ImmunoGen, Inc., Waltham, MA (US)

(72) Inventors: Kathleen R. Whiteman, Wilmington, MA (US); Paul Noordhuis, Leiden (NL); Yelena Kovtun, Stow, MA (US); Robert J. Lutz, Wayland, MA (US); Gerrit Jan Schuurhuis, Hoofddorp (NL); Russell Marlin Walker, Cambridge, MA (US)

(21) Appl. No.: 15/311,632

(22) PCT Filed: May 19, 2015
(86) PCT No.: PCT/US15/31580

§ 371 Date: Nov. 16, 2016

Related U.S. Application Data
(60) Provisional application No. 62/001,015, filed on May 20, 2014.
(60) Provisional application No. 62/011,456, filed on Jun. 12, 2014.
(60) Provisional application No. 62/075,715, filed on Nov. 5, 2014.

(51) Int. Cl. A61K 39/00; C12Q 1/68; A61K 31/5517; G01N 33/574

(52) U.S. Cl. CPC A61K 47/48384; A61K 47/4863; A61K 47/48715; A61K 31/5517; G01N 33/57426; C12Q 1/6886; C12Q 2600/158; G01N 2333/82; G01N 2333/70596

(57) ABSTRACT

The invention features methods for characterizing and treating acute myeloid leukemia (AML) (e.g., newly diagnosed, relapsed, and refractory AML) in a subject using immunoconjugates of the invention. In one aspect, the invention generally features a method of treating acute myeloid leukemia in a subject (e.g., a human), the method involving administering an effective amount of an immunoconjugate to a pre-selected subject, where the immunoconjugate contains a humanized or chimeric antibody or fragment conjugated to a cytotoxic benzodiazepine dimer compound via a cleavable disulfide linker.

### CROSS-REFERENCE TO RELATED APPLICATIONS

This application claims priority to and the benefit of U.S. Provisional Patent Application Ser. No. 62/001,015, filed May 20, 2014; 62/011,456, filed Jun. 12, 2014; and 62/075,715, filed Nov. 5, 2014, respectively. The entire contents of each of these applications is hereby incorporated by reference herein.

### BACKGROUND OF THE INVENTION

Acute myeloid leukemia (AML) is associated with the accumulation of abnormal blast cells in bone marrow. Acute myeloid leukemia (AML) is one of the most common types of leukemia among adults. In the United States alone, over 18,000 new cases of AML are identified each year, and more than 10,000 deaths are associated with AML. Despite high initial response rates to chemotherapy, many acute myeloid leukemia (AML) patients fail to achieve complete remission. In fact, the majority of patients with AML relapse within 3-5 years from diagnosis. AML relapse is thought to be due to the outgrowth of persistent leukemic stem cells (LSC). Accordingly, improved methods for characterizing AML in a subject and identifying an efficacious therapy, as well as improved methods for treating AML relapse, are urgently required.

### SUMMARY OF THE INVENTION

As described below, the present invention features methods for characterizing and treating acute myeloid leukemia (AML) (e.g., newly diagnosed, relapsed, and refractory AML) in a subject using immunoconjugates of the invention.

In one aspect, the invention generally features a method of treating acute myeloid leukemia in a subject (e.g., a human), the method involving administering an effective amount of an immunoconjugate to a pre-selected subject, where the immunoconjugate contains a humanized or chimeric antibody or fragment conjugated to a cytotoxic benzodiazepine dimer compound via a cleavable disulfide linker represented by the following structural formula:

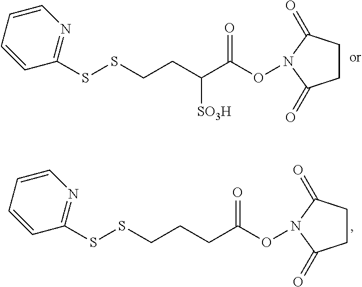 [SMILES: O=C(ON1C(=O)CCC1=O)C(CCSSc1ccccn1)S(=O)(=O)O ; O=C(CCCSSc1ccccn1)ON1C(=O)CCC1=O]

where the antibody contains a heavy chain variable region containing one or more complementarity determining regions that is any one or more of SEQ ID NOs: 1-3; and/or a light chain variable region containing one or more complementarity determining regions that is any one or more of SEQ ID NOs: 4-6; and the cytotoxic benzodiazepine dimer compound represented by one of the following structural formulas or a pharmaceutically acceptable salt thereof:

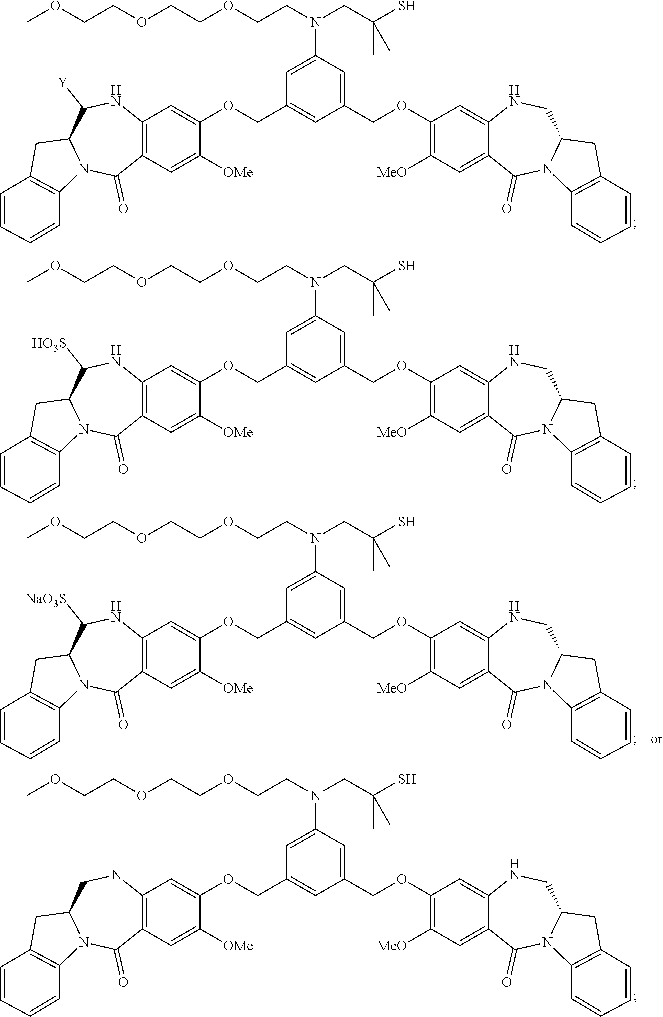

where Y is —SO₃M and M is H or a pharmaceutically acceptable cation and where the pre-selection involves detecting CD33 in a biological sample of the subject.

In another aspect, the invention features a method of treating acute myeloid leukemia in a subject, the method involving administering an effective amount of an immunoconjugate to a subject determined to have about 1,000 CD33 antigens per cell in a biological sample, where the immunoconjugate contains a humanized or chimeric antibody or fragment conjugated to a cytotoxic benzodiazepine dimer compound via a cleavable disulfide linker represented by the following structural formula:

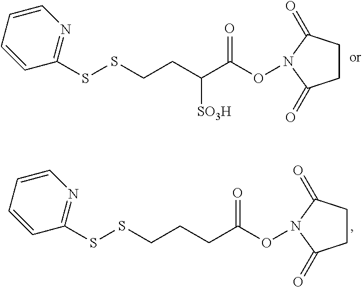 [SMILES: O=C(ON1C(=O)CCC1=O)C(CCSSc1ccccn1)S(=O)(=O)O ; O=C(CCCSSc1ccccn1)ON1C(=O)CCC1=O]

where the antibody contains a heavy chain variable region containing one or more complementarity determining regions that is any one or more of SEQ ID NOs: 1-3; and/or a light chain variable region containing one or more complementarity determining regions that is any one or more of SEQ ID NOs: 4-6; and the cytotoxic benzodiazepine dimer compound represented by one of the following structural formulas or a pharmaceutically acceptable salt thereof:

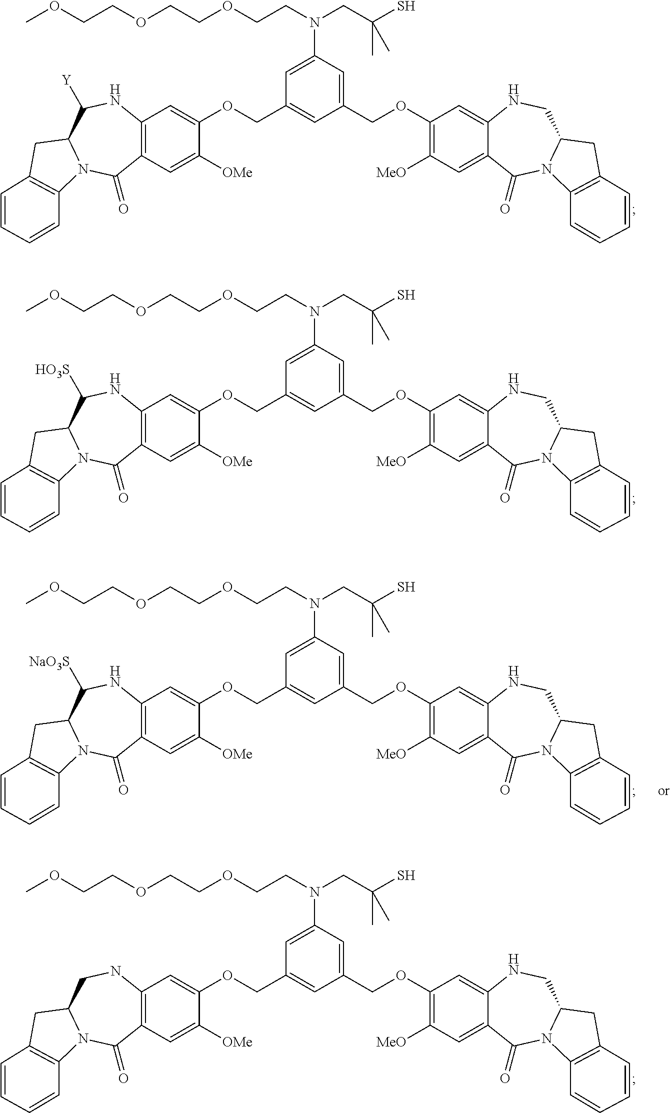

where Y is —SO₃M and M is H or a pharmaceutically acceptable cation and where the pre-selection involves detecting CD33 in a biological sample of the subject.

In another aspect, the invention features a method of treating a subject having FLT3-ITD positive acute myeloid leukemia, the method involving administering an effective amount of an immunoconjugate to a pre-selected subject, where the immunoconjugate contains a humanized or chimeric antibody or fragment conjugated to a cytotoxic benzodiazepine dimer compound via a cleavable disulfide linker represented by the following structural formula:

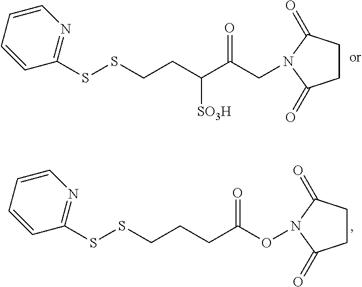 [SMILES: O=C(CN1C(=O)CCC1=O)C(CCSSc1ccccn1)S(=O)(=O)O ; O=C(CCCSSc1ccccn1)ON1C(=O)CCC1=O]

where the antibody contains a heavy chain variable region containing one or more complementarity determining regions that is any one or more of SEQ ID NOs: 1-3; and/or a light chain variable region containing one or more complementarity determining regions that is any one or more of SEQ ID NOs: 4-6; and the cytotoxic benzodiazepine dimer compound represented by one of the following structural formulas or a pharmaceutically acceptable salt thereof:

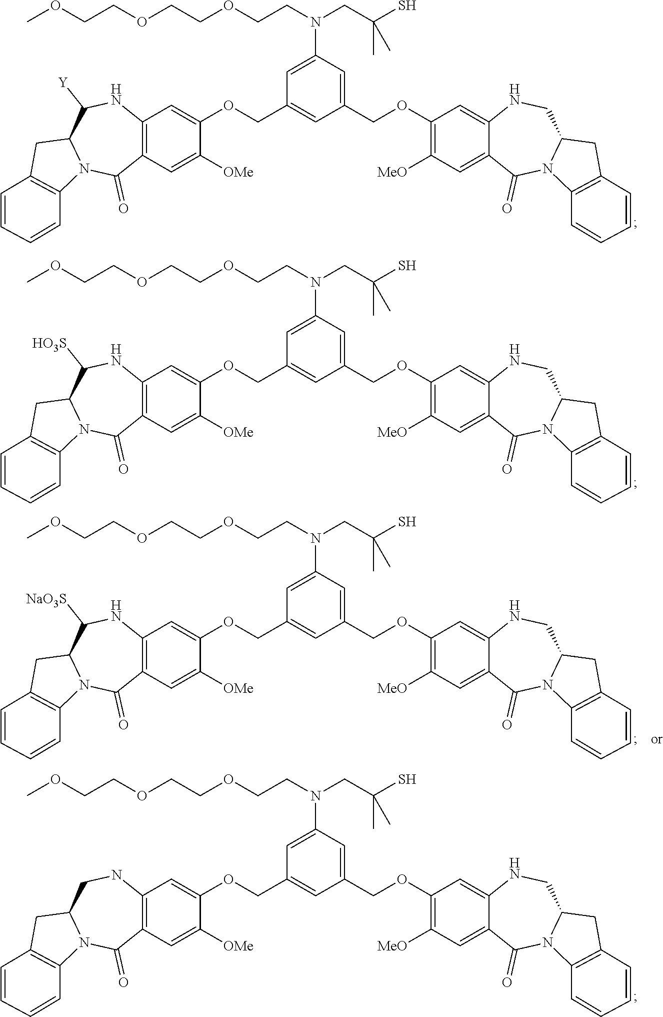

where Y is —SO₃M and M is H or a pharmaceutically acceptable cation and where the pre-selection comprises detecting FLT3-ITD in a biological sample of the subject.

In another aspect, the invention features a method of treating a subject having acute myeloid leukemia, the method comprising administering an effective amount of an immunoconjugate to a pre-selected subject determined to have FLT3-ITD positive acute myeloid leukemia, where the immunoconjugate contains a humanized or chimeric antibody or fragment conjugated to a cytotoxic benzodiazepine dimer compound via a cleavable disulfide linker represented by the following structural formula:

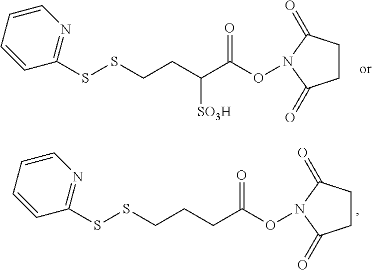

where the antibody contains a heavy chain variable region containing one or more complementarity determining regions that is any one or more of SEQ ID NOs: 1-3; and/or a light chain variable region containing one or more complementarity determining regions that is any one or more of SEQ ID NOs: 4-6; and the cytotoxic benzodiazepine dimer compound represented by one of the following structural formulas or a pharmaceutically acceptable salt thereof:

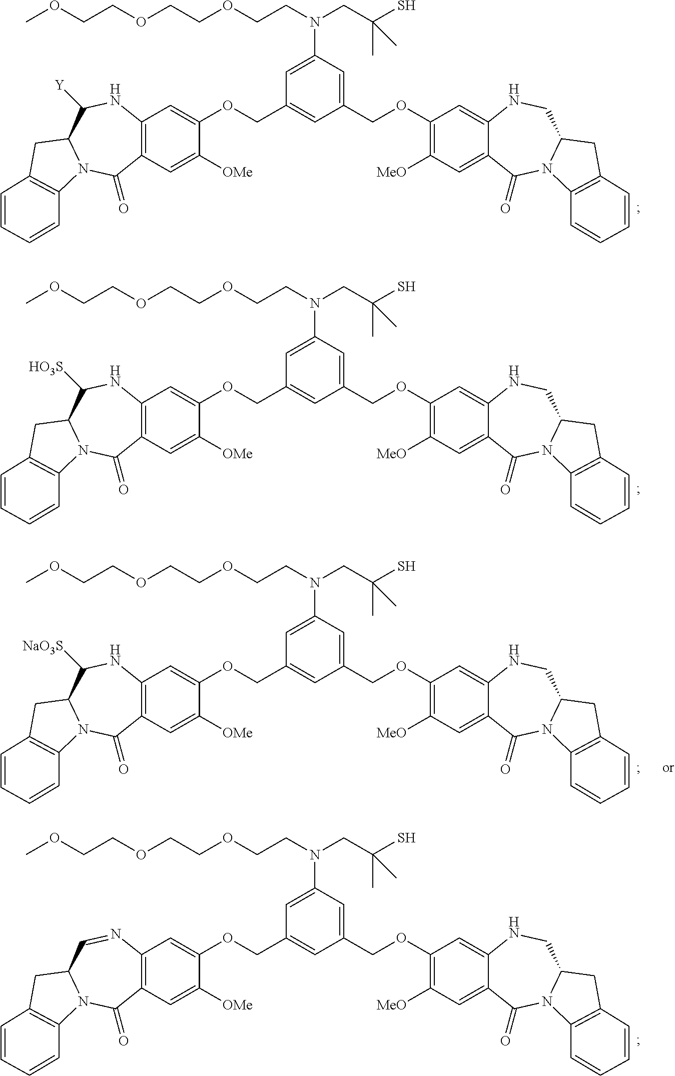

where Y is —SO₃M and M is H or a pharmaceutically acceptable cation and where the pre-selection comprises determining the FLT3-ITD status in a biological sample of the subject.

In another aspect, the invention features a method of identifying a subject as being responsive to treatment with an immunoconjugate, the method involving: detecting FLT3-ITD in a biological sample from the subject, and correlating the detection of FLT3-ITD with responsiveness of the subject to treatment, where the presence of FLT3-ITD in the biological sample identifies the subject as responsive to treatment with the immunoconjugate,

where the immunoconjugate contains a humanized or chimeric antibody or fragment conjugated to a cytotoxic benzodiazepine dimer compound via a cleavable disulfide linker represented by the following structural formula:

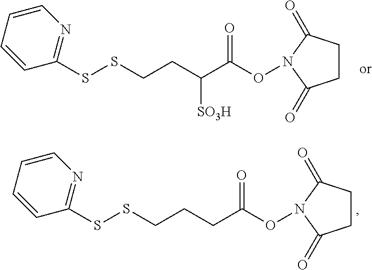

where the antibody contains a heavy chain variable region containing one or more complementarity determining regions that is any one or more of SEQ ID NOs: 1-3; and/or a light chain variable region containing one or more complementarity determining regions that is any one or more of SEQ ID NOs: 4-6; and the cytotoxic benzodiazepine dimer compound represented by one of the following structural formulas or a pharmaceutically acceptable salt thereof:

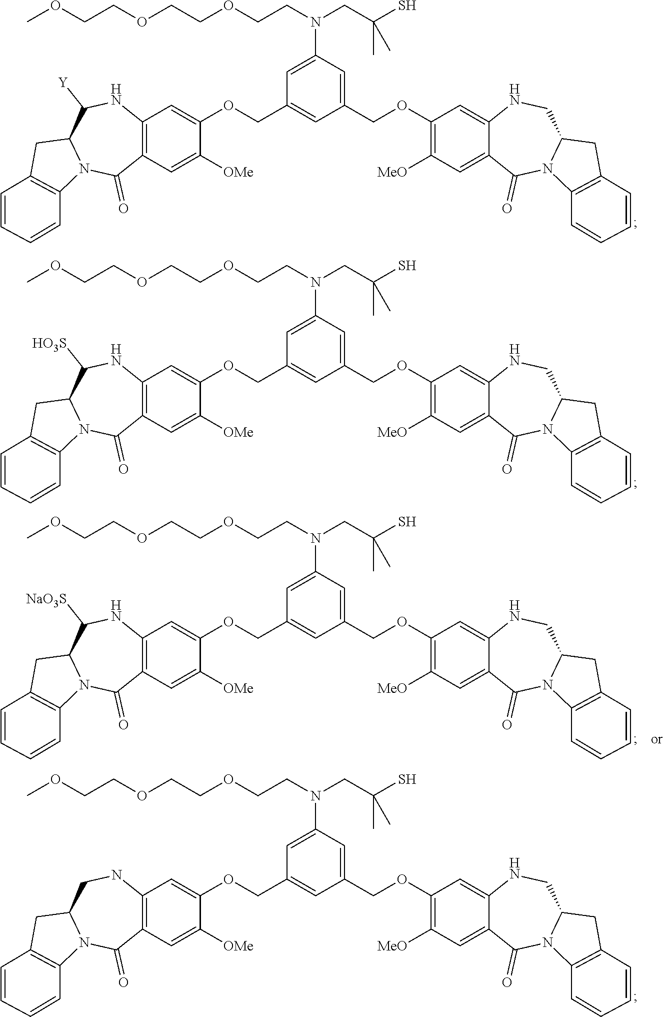

where Y is —SO₃M and M is H or a pharmaceutically acceptable cation.

In another aspect, the invention features a method for treating or preventing acute myeloid leukemia relapse in a subject, the method involving administering an effective amount of an immunoconjugate to a pre-selected subject determined to have FLT3-ITD positive acute myeloid leukemia and that has not received prior treatment with a tyrosine kinase inhibitor, where the immunoconjugate contains a humanized or chimeric antibody or fragment conjugated to a cytotoxic benzodiazepine dimer compound via a cleavable disulfide linker represented by the following structural formula:

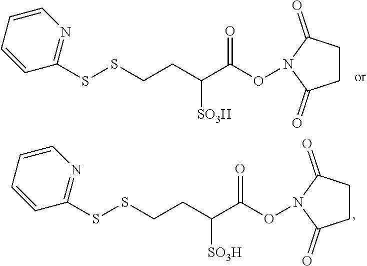

where the antibody contains a heavy chain variable region containing one or more complementarity determining regions that is any one or more of SEQ ID NOs: 1-3; and/or a light chain variable region containing one or more complementarity determining regions that is any one or more of SEQ ID NOs: 4-6; and the cytotoxic benzodiazepine dimer compound represented by one of the following structural formulas or a pharmaceutically acceptable salt thereof:

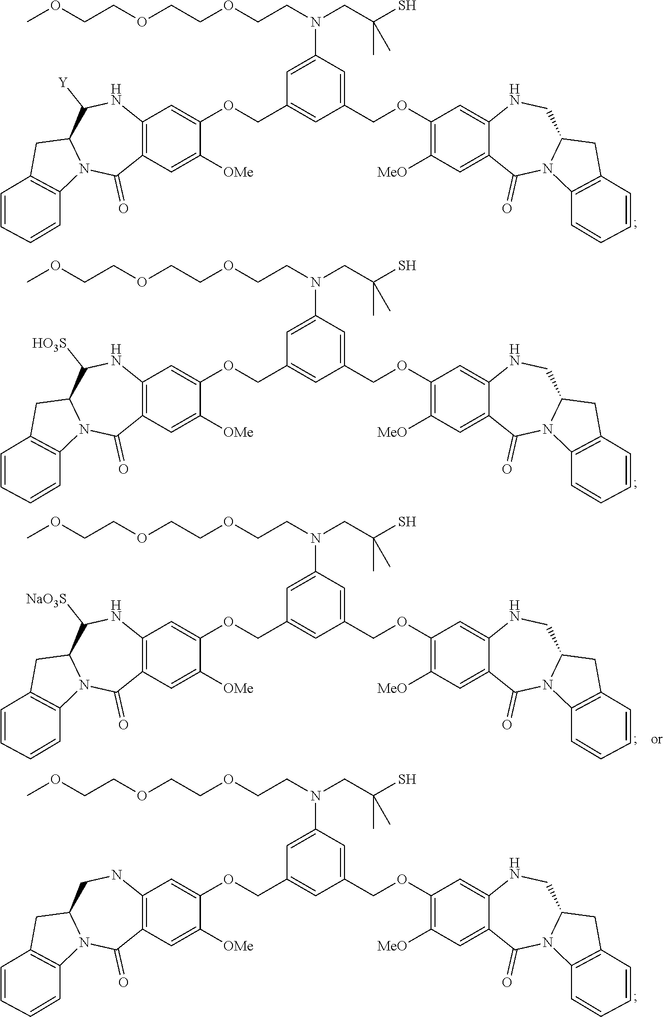

where Y is —SO₃M and M is H or a pharmaceutically acceptable cation.

In another aspect, the invention features a method for treating or preventing acute myeloid leukemia relapse in a subject, the method involving administering an effective amount of an immunoconjugate to a pre-selected subject determined to have FLT3-ITD positive acute myeloid leukemia and that has received prior treatment with a tyrosine kinase inhibitor, where the immunoconjugate contains a humanized or chimeric antibody or fragment conjugated to a cytotoxic benzodiazepine dimer compound via a cleavable disulfide linker represented by the following structural formula:

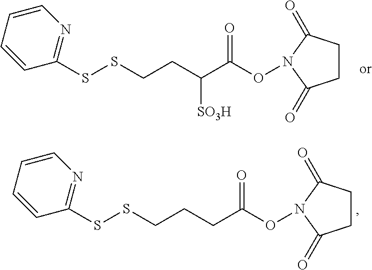

where the antibody contains a heavy chain variable region containing one or more complementarity determining regions that is any one or more of SEQ ID NOs: 1-3; and/or a light chain variable region containing one or more complementarity determining regions that is any one or more of SEQ ID NOs: 4-6; and the cytotoxic benzodiazepine dimer compound represented by one of the following structural formulas or a pharmaceutically acceptable salt thereof:

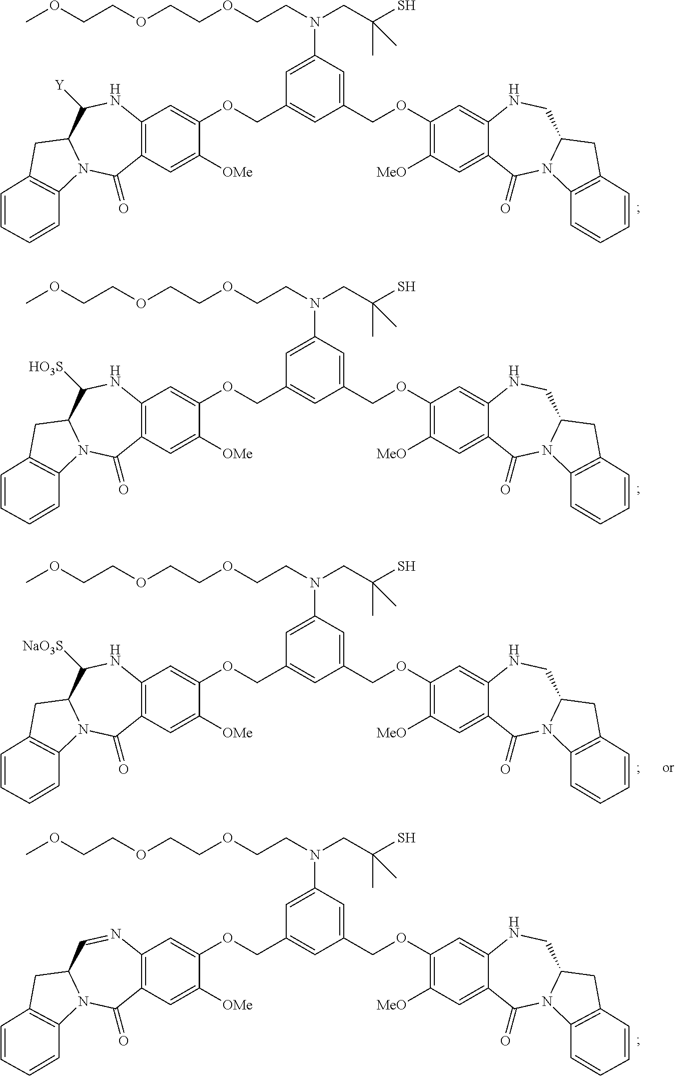

where Y is —SO₃M and M is H or a pharmaceutically acceptable cation.

In another aspect, the invention features a method for treating a subject having multi-drug resistant acute myeloid leukemia, the method involving administering an effective amount of an immunoconjugate to a subject, where the immunoconjugate contains a humanized or chimeric antibody or fragment conjugated to a cytotoxic benzodiazepine dimer compound via a cleavable disulfide linker represented by the following structural formula:

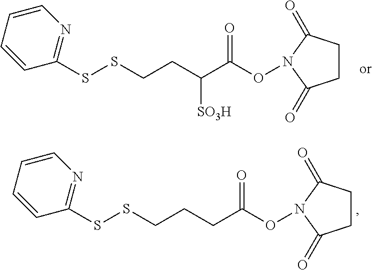

where the antibody contains a heavy chain variable region containing one or more complementarity determining regions that is any one or more of SEQ ID NOs: 1-3; and/or a light chain variable region containing one or more complementarity determining regions that is any one or more of SEQ ID NOs: 4-6; and the cytotoxic benzodiazepine dimer compound represented by one of the following structural formulas or a pharmaceutically acceptable salt thereof:

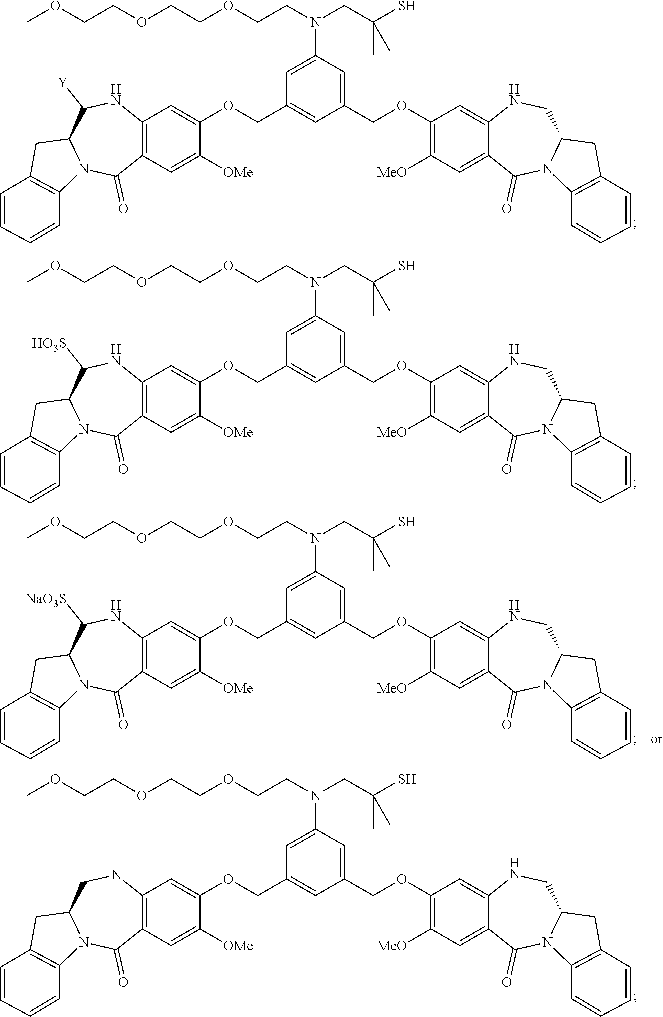

where Y is —SO₃M and M is H or a pharmaceutically acceptable cation, thereby treating the multi-drug resistant acute myeloid leukemia. In one embodiment, the subject is identified as having multi-drug resistant leukemia. In another embodiment, the subject is identified as having multi-drug resistant leukemia by detecting the presence of P-glycoprotein expression in a peripheral blood or bone marrow sample of the subject. In yet another embodiment, the method further involves detecting the presence of CD33 expression in a peripheral blood or bone marrow sample of the subject. In yet another embodiment, a level greater than about 1,000, 3,000, or 5,000 CD33 antigens per cell identifies the AML as responsive to treatment with the immunoconjugate.

In yet another aspect, the invention features a method for treating or preventing acute myeloid leukemia relapse in a subject, involving administering an effective amount of an immunoconjugate to the subject, where the immunoconjugate contains a humanized or chimeric antibody or fragment conjugated to a cytotoxic benzodiazepine dimer compound via a cleavable disulfide linker represented by the following structural formula:

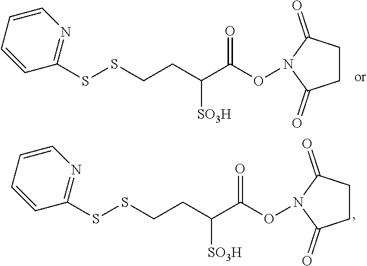

where the antibody contains a heavy chain variable region containing one or more complementarity determining regions that is any one or more of SEQ ID NOs: 1-3; and/or a light chain variable region containing one or more complementarity determining regions that is any one or more of SEQ ID NOs: 4-6; and the cytotoxic benzodiazepine dimer compound represented by one of the following structural formulas or a pharmaceutically acceptable salt thereof:

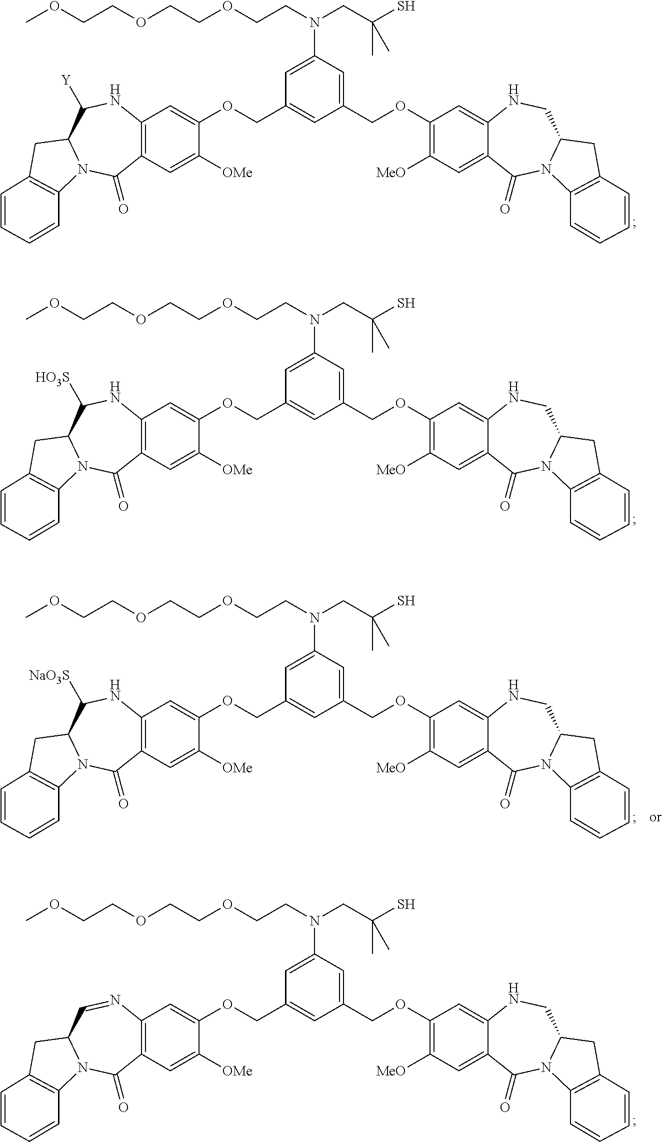

where Y is —SO₃M and M is H or a pharmaceutically acceptable cation, thereby treating the acute myeloid leukemia relapse. In one embodiment, the method prevents, reduces, or eliminates minimal residual disease. In another embodiment, the antibody specifically binds a CD33-expressing leukemic progenitor and/or leukemic stem cell. In another embodiment, the method spares normal hematopoietic stem cells.

In another aspect, the invention features a method for inducing cell death in a leukemic stem cell, the method involving contacting the leukemic stem cell with an effective amount of an immunoconjugate containing a humanized or chimeric antibody or fragment conjugated to a cytotoxic benzodiazepine dimer compound via a cleavable disulfide linker represented by the following structural formula:

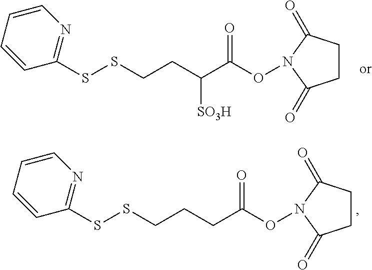

where the antibody contains a heavy chain variable region containing one or more complementarity determining regions that is any one or more of SEQ ID NOs: 1-3; and/or a light chain variable region containing one or more complementarity determining regions that is any one or more of SEQ ID NOs: 4-6; and the cytotoxic benzodiazepine dimer compound represented by one of the following structural formulas or a pharmaceutically acceptable salt thereof:

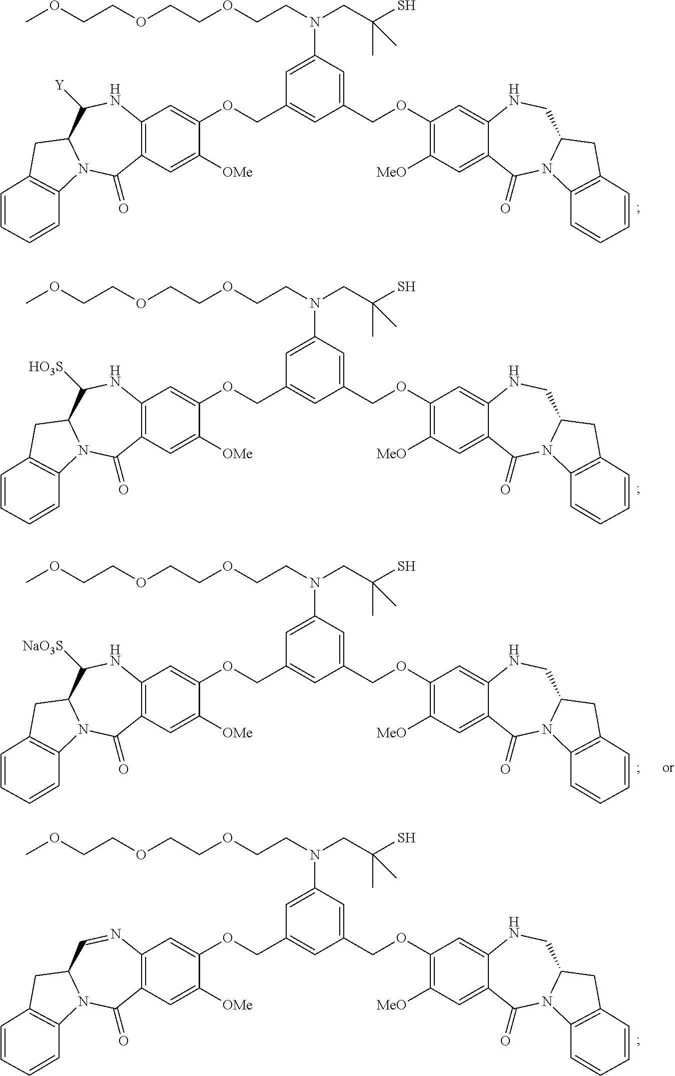

where Y is —SO₃M and M is H or a pharmaceutically acceptable cation, thereby inducing cell death in the leukemic stem cell. In one embodiment, the method does not induce cell death in a normal hematopoietic stem cell. In another embodiment, the contacting is in vitro or in vivo. In another embodiment, the leukemic stem cell is in a subject newly diagnosed with acute myeloid leukemia, in a subject identified as having a relapse associated with the growth or proliferation of a leukemic stem cell, or in a subject identified as having refractory acute myeloid leukemia.

In another aspect, the invention features a method for inducing cell death in a FLT3-ITD positive leukemic cell, the method involving contacting the leukemic stem cell with an effective amount of an immunoconjugate containing a humanized or chimeric antibody or fragment conjugated to a cytotoxic benzodiazepine dimer compound via a cleavable disulfide linker represented by the following structural formula:

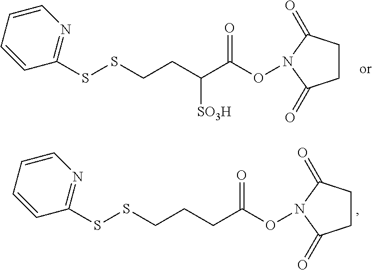

where the antibody contains a heavy chain variable region containing one or more complementarity determining regions that is any one or more of SEQ ID NOs: 1-3; and/or a light chain variable region containing one or more complementarity determining regions that is any one or more of SEQ ID NOs: 4-6; and the cytotoxic benzodiazepine dimer compound represented by one of the following structural formulas or a pharmaceutically acceptable salt thereof:

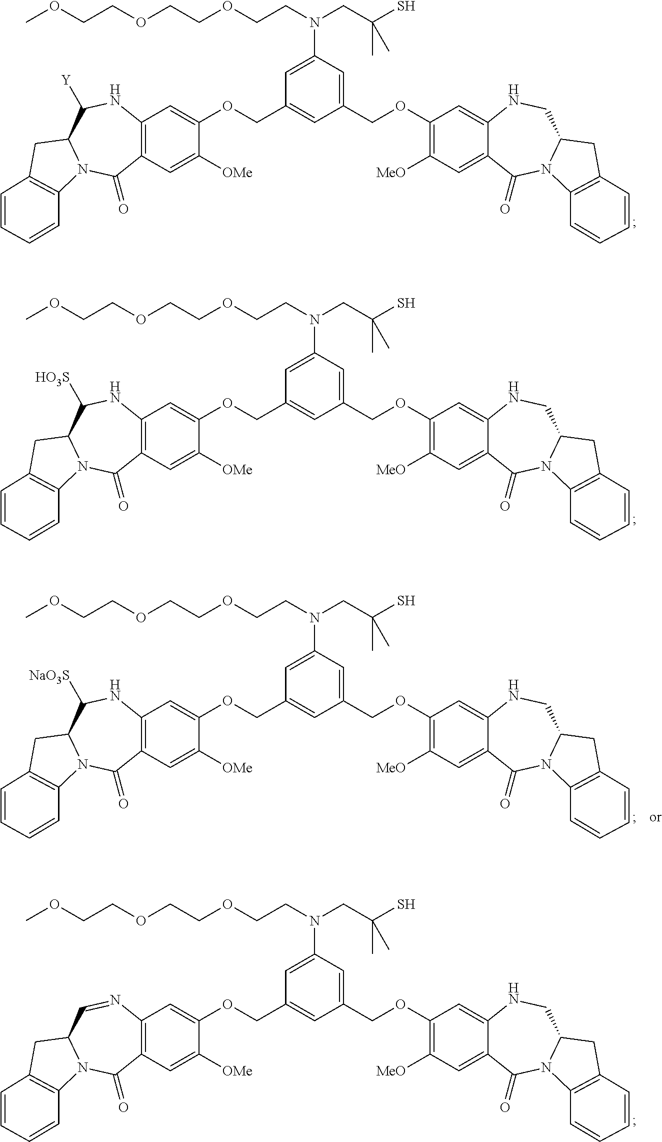

where Y is —SO₃M and M is H or a pharmaceutically acceptable cation, thereby inducing cell death in the FLT3-ITD positive leukemic cell. In one embodiment, the method does not induce cell death in a normal hematopoietic stem cell. In another embodiment, the contacting is in vitro or in vivo. In another embodiment, the leukemic stem cell is in a subject newly diagnosed with acute myeloid leukemia, in a subject identified as having a relapse associated with the growth or proliferation of a leukemic stem cell, or in a subject identified as having refractory acute myeloid leukemia.

In another aspect, the invention features a kit containing an anti-CD33 antibody and a therapeutic composition containing an effective amount of an immunoconjugate containing a humanized My9-6 antibody linked by N-succinimidyl-4-(2-pyridyldithio)-2-sulfobutanoate to a cytotoxic benzodiazepine dimer compound, where the immunoconjugate is represented by one of the following structural formulas or a pharmaceutically acceptable salt thereof:

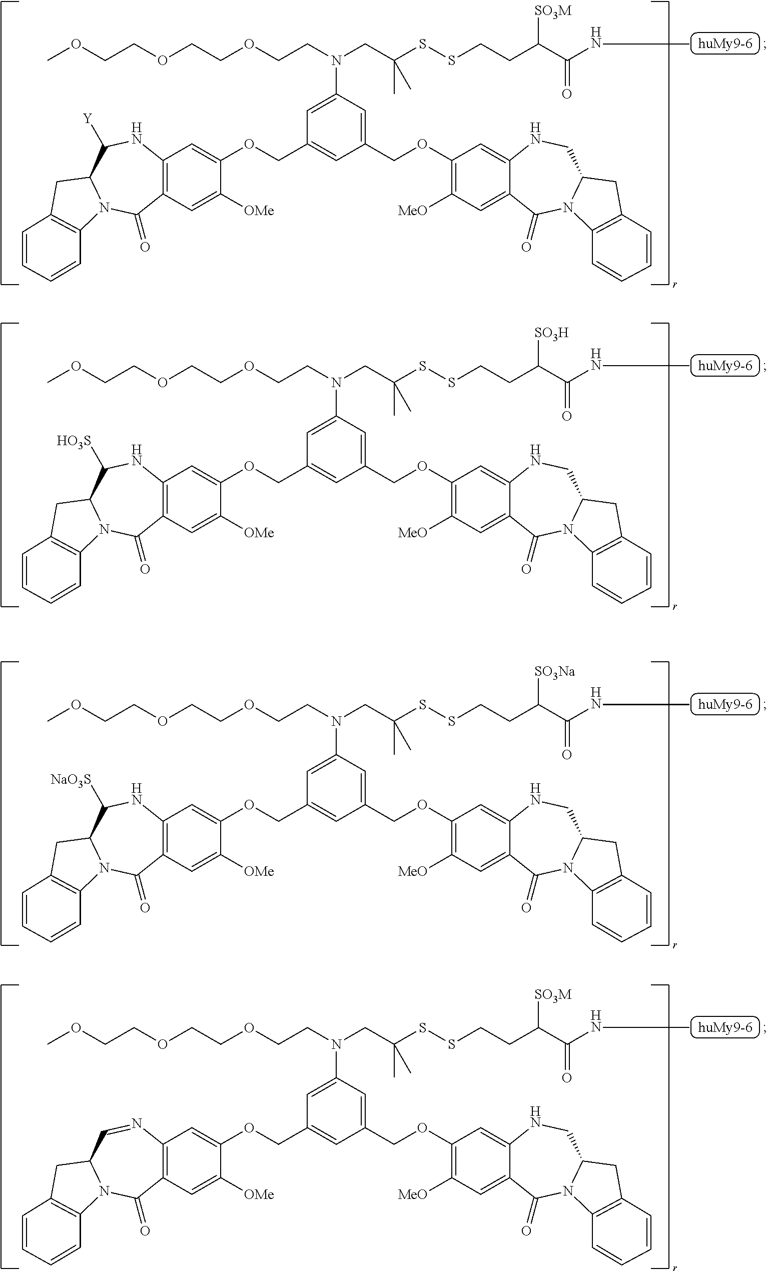

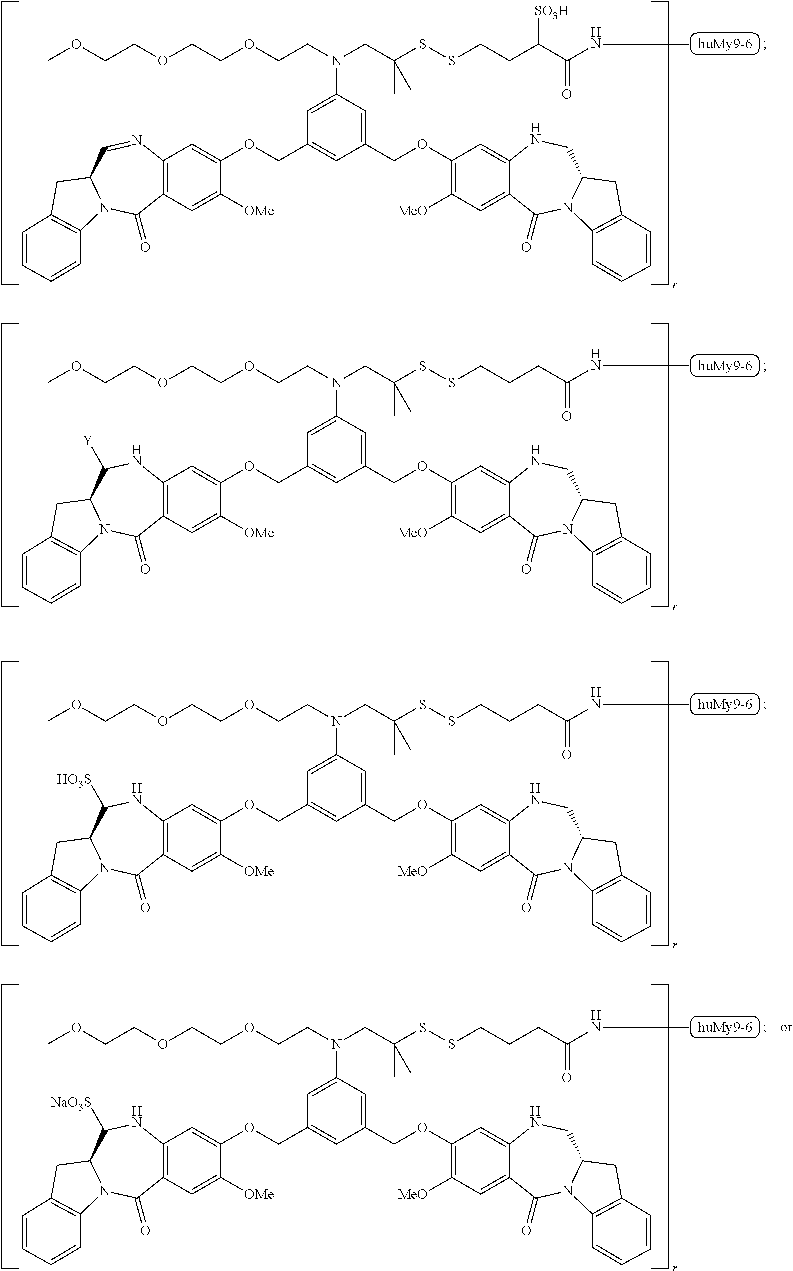

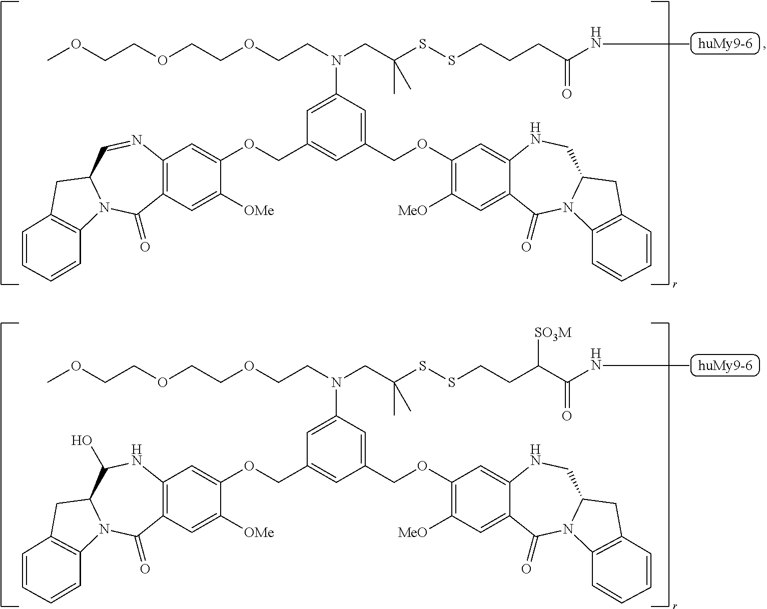

wherein r is an integer from 1 to 10, Y is —SO₃M and M, for each occurrence, is independently —H or a pharmaceutically acceptable cation. In one embodiment, the kit further contains directions for detecting the level of CD33 expression in a sample from a subject using the anti-CD33 antibody. In another embodiment, further containing instructions for administering the immunoconjugate to a subject identified as having at least about 1,000 antigens per cell. In another embodiment, the subject is identified as having at least about 3,000 or 5,000 antigens per cell.

In various embodiments of the above aspects, or any other aspect of the invention delineated herein, the heavy chain variable region contains an amino acid sequence having at least 95% identity to the amino acid sequence of SEQ ID NO:7 or 9 and the light chain variable region contains an amino acid sequence having at least 95% identity to the amino acid sequence of SEQ ID NO: 8 or 10. In various embodiments of the above aspects, the antibody antibody has at least one heavy chain variable region or fragment thereof containing three sequential complementarity-determining regions having the amino acid sequences set forth in SEQ ID NOs:1-3, respectively, and at least one light chain variable region or fragment thereof containing three sequential complementarity-determining regions having amino acid sequences set forth in SEQ ID NOs:4-6, respectively. In various embodiments of the above aspects, the antibody or fragment thereof has a heavy chain variable region CDR1 having the amino acid sequence of SEQ ID NO:1; a heavy chain variable region CDR2 having the amino acid sequence of SEQ ID NO:2; a heavy chain variable region CDR3 having the amino acid sequence of SEQ ID NO:3; a light chain variable region CDR1 having the amino acid sequence of SEQ ID NO:4; a light chain variable region CDR2 having the amino acid sequence of SEQ ID NO:5; and a light chain variable region CDR3 having the amino acid sequence of SEQ ID NO:6. In various embodiments of the above aspects, the antibody is a humanized or chimeric My9-6 antibody. In various embodiments of the above aspects, the humanized antibody is a CDR-grafted or resurfaced antibody. In various embodiments of the above aspects, the immunoconjugate contains a humanized My9-6 antibody conjugated to a cytotoxic benzodiazepine dimer compound via N-succinimidyl-4-(2-pyridyldithio)-2-sulfobutanoate, where the immunoconjugate is represented by one of the following structural formulas or a pharmaceutically acceptable salt thereof:

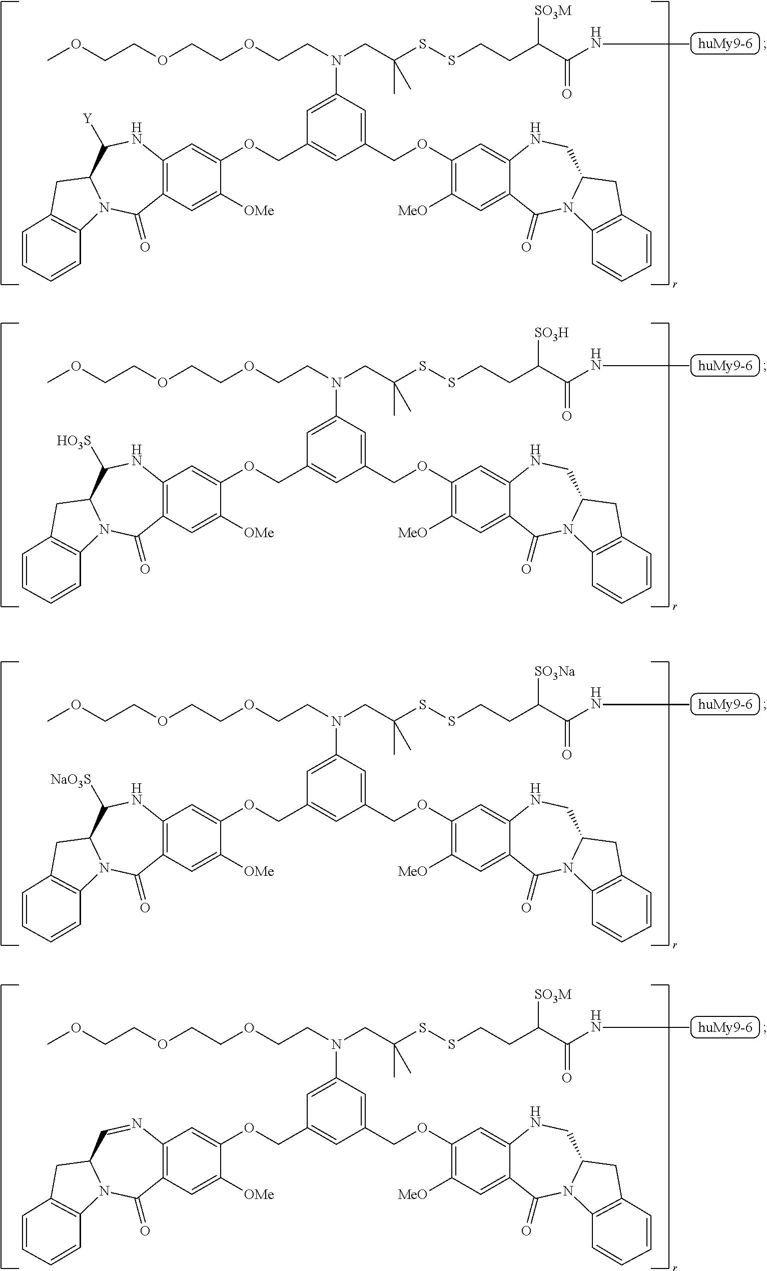

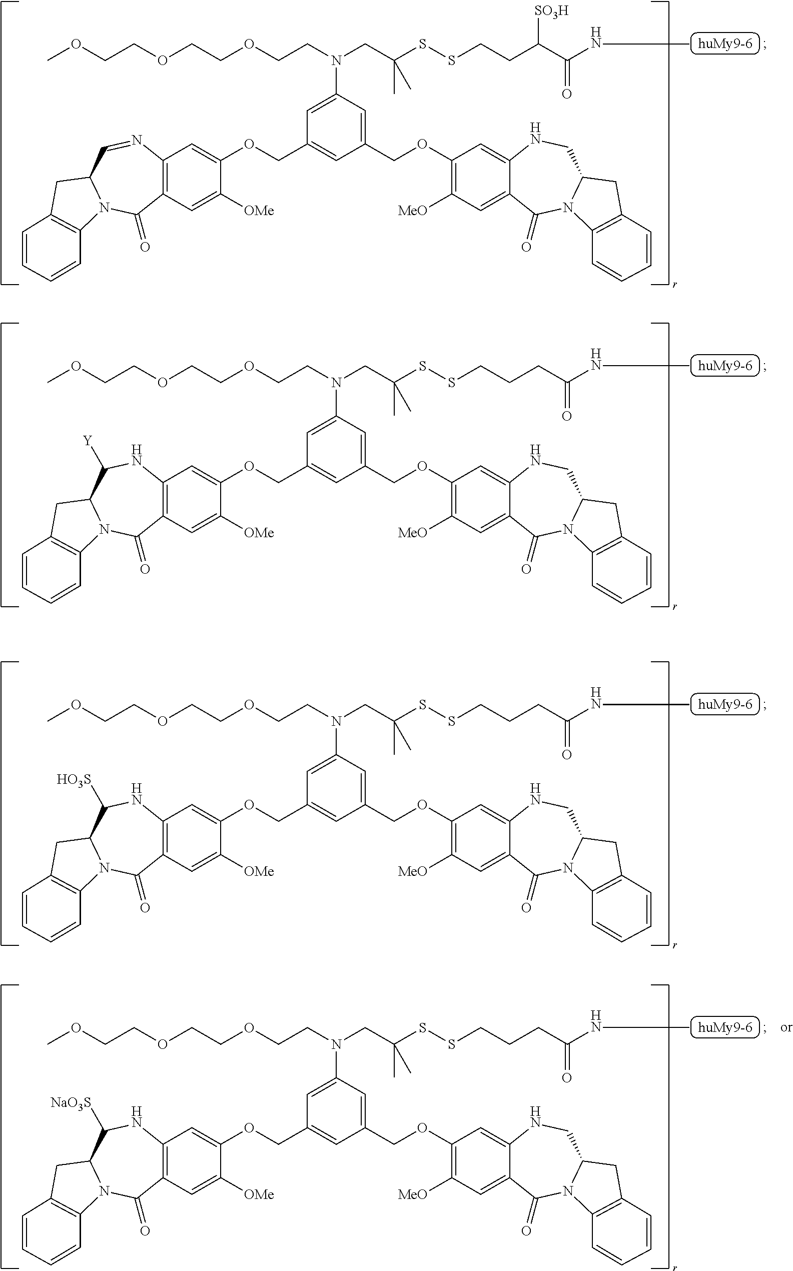

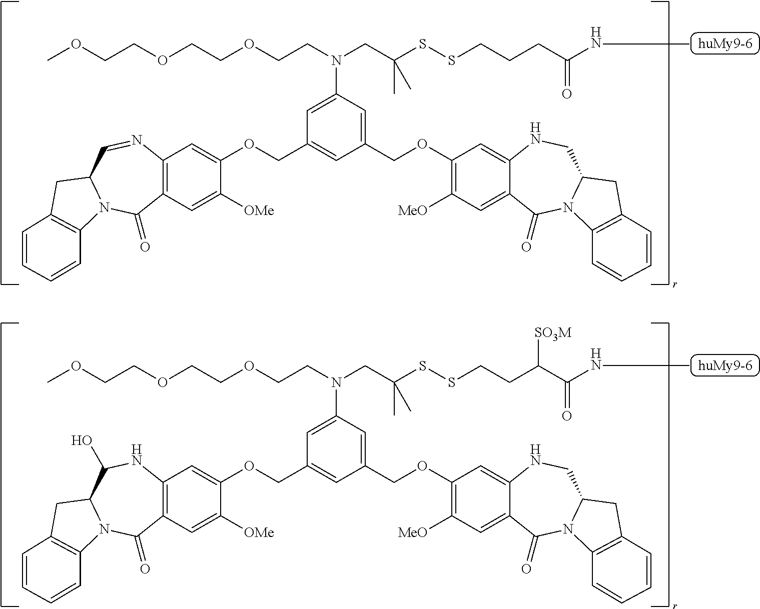

where r is an integer from 1 to 10, Y is —SO₃M and M, for each occurrence, is independently —H or a pharmaceutically acceptable cation.

In various embodiments of the above aspects, the detecting step involves measuring the level of CD33 present in a peripheral blood or bone marrow sample of the subject, where detecting between about 1,000-25,000 (e.g., 2,000-20,000; 3,000-25,000; 3,000-20,000; 3,000-18,000; 5,000-18,000; 5,000-20,000; 5,000-25,000) antigens per cell pre-selects the subject as likely to respond to the immunoconjugate. In various embodiments of the above aspects, detecting between about 3,000-25,000 antigens per cell pre-selects the subject as likely to respond to the immunoconjugate or detecting between about 5,000-25,000 antigens per cell pre-selects the subject as likely to respond to the immunoconjugate. In various embodiments of the above aspects, the detecting step involves measuring the level of CD33 present in a peripheral blood or bone marrow sample of the subject, where detecting at least about 1,000, 3,000, or 5,000 antigens per cell pre-selects the subject as likely to respond to the immunoconjugate. In various embodiments of the above aspects, the subject is newly diagnosed with acute myeloid leukemia. In various embodiments of the above aspects, the subject is diagnosed with acute myeloid leukemia relapse or with refractory acute myeloid leukemia. In various embodiments of the above aspects, a sample from the subject diagnosed with acute myeloid leukemia relapse or with refractory acute myeloid leukemia contains at least about 3,000 antigens per cell. In various embodiments of the above aspects, the immunoconjugate has an IC₅₀ value from about 10 pM to about 2 nM. In various embodiments of the above aspects, the immunoconjugate has an IC₅₀ value from about 11 pM to about 1.6 nM. In various embodiments of the above aspects, the method preferentially kills leukemic stem cells.

In various embodiments of the above aspects, or any other aspect of the invention delineated herein, the detecting step involves detecting the presence of a FLT3-ITD mutation in a biological (e.g., peripheral blood or bone marrow) sample of the subject. In various embodiments of the above aspects, the detecting step involves a nucleic acid hybridization method or a nucleic acid sequencing method. In various embodiments of the above aspects, the detecting step involves one or more of PCR, reverse transcriptase PCR, or real time PCR. In various embodiments of the above aspects, the tyrosine kinase inhibitor is a FLT3 tyrosine kinase inhibitor. In various embodiments of the above aspects, a subject having FLT3-ITD positive acute myeloid leukemia is diagnosed with acute myeloid leukemia relapse and has not received prior treatment with a tyrosine kinase inhibitor (e.g., FLT3 tyrosine kinase inhibitor). In various embodiments of the above aspects, a subject having FLT3-ITD positive acute myeloid leukemia is diagnosed with acute myeloid leukemia relapse after receiving prior treatment with a tyrosine kinase inhibitor (e.g., FLT3 tyrosine kinase inhibitor). In various embodiments of the above aspects, a subject having FLT3-ITD positive acute myeloid leukemia is diagnosed with refractory acute myeloid leukemia and has not received prior treatment with a tyrosine kinase inhibitor (e.g., FLT3 tyrosine kinase inhibitor). In various embodiments of the above aspects, a subject having FLT3-ITD positive acute myeloid leukemia is diagnosed with refractory acute myeloid leukemia after receiving prior treatment with a tyrosine kinase inhibitor (e.g., FLT3 tyrosine kinase inhibitor).

In certain embodiments of any of the above-aspects, a composition comprising the conjugates described herein may comprise an average 1-10 cytotoxic benzodiazepine dimer molecule per antibody molecule. The average ratio of cytotoxic benzodiazepine dimer molecule per antibody molecule is referred to herein as the Drug Antibody Ratio (DAR). In one embodiment, the DAR is between 2-8, 3-7, 3-5 or 2.5-3.5.

Other features and advantages of the invention will be apparent from the detailed description, and from the claims.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “P-glycoprotein” is meant a polypeptide or fragment thereof having at least about 85% amino acid sequence identity to the human sequence provided at NCBI Accession No. NP_001035830 and conferring multi-drug resistance on a cell in which it is expressed. The sequence of an exemplary human P-glycoprotein is provided below:

| | |
|---|---|
| 1 | maaaeaggdd arcvrlsaer aqalladvdt llfdcdgvlw rgetavpgap ealralrarg |
| 61 | krlgfitnns sktraayaek lrrlgfggpa gpgaslevfg tayctalylr qrlagapapk |
| 121 | ayvlgspala aeleavgvas vgvgpeplqg egpgdwlhap lepdvravvv gfdphfsymk |
| 181 | ltkalrylqq pgcllvgtnm dnrlplengr fiagtgclvr avemaaqrqa diigkpsrfi |
| 241 | fdcvsqeygi npertvmvgd rldtdillga tcglktiltl tgvstlgdvk nnqesdcvsk |
| 301 | kkmvpdfyvd siadllpalq g |

By “P-glycoprotein polynucleotide” is meant a nucleic acid molecule encoding P-glycoprotein.

By “CD33 protein” is meant a polypeptide or fragment thereof having at least about 85% amino acid sequence identity to the human sequence provided at NCBI Accession No. CAD36509 and having anti-CD33 antibody binding activity. An exemplary human CD33 amino acid sequence is provided below:

| | |
|---|---|
| 1 | mplllllpll wagalamdpn fwlqvqesvt vqeglcvlvp ctffhpipyy dknspvhgyw |
| 61 | fregaiisrd spvatnkldq evqeetqgrf rllgdpsrnn cslsivdarr rdngsyffrm |
| 121 | ergstkysyk spqlsvhvtd lthrpkilip gtlepghskn ltcsvswace qgtppifswl |
| 181 | saaptslgpr tthssvliit prpqdhgtnl tcqvkfagag vttertiqln vtyvpqnptt |
| 241 | gifpgdgsgk qetragvvhg aiggagvtal lalclcliff ivkthrrkaa rtavgrndth |
| 301 | pttgsaspkh qkksklhgpt etsscsgaap tvemdeelhy aslnfhgmnp skdtsteyse |
| 361 | vrtq |

By “CD33 polynucleotide” is meant a nucleic acid molecule encoding a CD33 protein.

By “FLT3 protein,” “FLT3 polypeptide,” “FLT3,” “FLT-3 Receptor,” or “FLT-3R” is meant a polypeptide or fragment thereof having at least about 85%, 90%, 95%, 99% or 100% amino acid sequence identity to the human sequence of FLT3 tyrosine kinase receptor, also referred to as FLK-2 and STK-1, provided at NCBI Accession No. NP_004110 and having tyrosine kinase activity, including receptor tyrosine kinase activity. In one embodiment the FLT3 amino acid sequence is the human FLT3 amino acid sequence provided below:

| | |
|---|---|
| 1 | mpalardggq lpllvvfsam ifgtitnqdl pvikcvlinh knndssvgks ssypmvsesp |
| 61 | edlgcalrpq ssgtvyeaaa vevdvsasit lqvlvdapgn isclwvfkhs slncqphfdl |
| 121 | qnrgvvsmvi lkmtetqage yllfiqseat nytilftvsi rntllytlrr pyfrkmenqd |
| 181 | alvcisesvp epivewvlcd sqgesckees pavvkkeekv lhelfgtdir ccarnelgre |
| 241 | ctrlftidln qtpqttlpql flkvgeplwi rckavhvnhg fgltwelenk aleegnyfem |
| 301 | stystnrtmi rilfafvssv arndtgyytc ssskhpsqsa lvtivekgfi natnssedye |
| 361 | idqyeefcfs vrfkaypqir ctwtfsrksf pceqkgldng ysiskfcnhk hqpgeyifha |
| 421 | enddaqftkm ftlnirrkpq vlaeasasqa scfsdgyplp swtwkkcsdk spncteeite |
| 481 | gvwnrkanrk vfgqwvssst lnmseaikgf lvkccaynsl gtscetilln spgpfpfiqd |
| 541 | nisfyatigv cllfivvltl lichkykkqf ryesqlqmvq vtgssdneyf yvdfreyeyd |
| 601 | lkwefprenl efgkvlgsga fgkvmnatay gisktgvsiq vavkmlkeka dsserealms |
| 661 | elkmmtqlgs henivnllga ctlsgpiyli feyccygdll nylrskrekf hrtwteifke |
| 721 | hnfsfyptfq shpnssmpgs revqihpdsd qisglhgnsf hsedeieyen qkrleeeedl |
| 781 | nvltfedllc fayqvakgme flefkscvhr dlaarnvlvt hgkvvkicdf glardimsds |
| 841 | nyvvrgnarl pvkwmapesl fegiytiksd vwsygillwe ifslgvnpyp gipvdanfyk |
| 901 | liqngfkmdq pfyateeiyi imqscwafds rkrpsfpnlt sflgcqlada eeamyqnvdg |
| 961 | rvsecphtyq nrrpfsremd lgllspqaqv eds |

By “FLT3-ITD” is meant a FLT3 polypeptide having internal tandem duplication(s) including but not limited to simple tandem duplication(s) and/or tandem duplication(s) with insertion. In various embodiments, FLT3 polypeptides having internal tandem duplications are activated FLT3 variants (e.g., constitutively autophosphorylated). In some embodiments, the FLT3-ITD includes tandem duplications and/or tandem duplication(s) with insertion in any exon or intron including, for example, exon 11, exon 11 to intron 11, and exon 12, exon 14, exon 14 to intron 14, and exon 15. The internal tandem duplication mutation (FLT3-ITD) is the most common FLT3 mutation, present in about 20-25% of AML cases. Patients with FLT3-ITD AML have a worse prognosis than those with wild-type (WT) FLT3, with an increased rate of relapse and a shorter duration of response to chemotherapy.

By “FLT3 polynucleotide” is meant a nucleic acid molecule encoding a FLT3 protein.

By “leukemic stem cell” is meant a leukemia cell capable of self-renewal, capable of initiating leukemia, and/or capable of triggering acute myeloid leukemia relapse in a subject.

By “multi-drug resistant cell” is meant that a cell has a reduced response to one or more agents relative to the response of a control cell. In particular, a cell expressing P-glycoprotein is predicted to be less responsive to treatment with chemotherapeutics than a control cell.

By “ameliorate” is meant decrease, suppress, attenuate, diminish, arrest, or stabilize the development or progression of a disease.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “disease” is meant any condition or disorder that damages or interferes with the normal function of a cell, tissue, or organ. An example of a disease is acute myeloid leukemia, myelodysplastic syndrome (MDS), Acute Promyelocytic Leukemia (APL), chronic myeloid leukemia (CML).

By “effective amount” is meant the amount of a compound or agent required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician will decide the appropriate amount and dosage regimen. Such amount is referred to as an “effective” amount.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid molecule (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “reference” is meant a standard or control condition or sample.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “specifically binds” is meant an antibody or fragment thereof that recognizes and binds a polypeptide of interest, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 is a graph showing CD33 levels in patient acute myeloid leukemia (AML) samples (n=56).

FIG. 2 is a scatter plot showing a comparison of the in vitro IC₅₀ values and dependence on CD33 expression level for IMGN779 compared with a CD33-targeting maytansinoid antibody drug conjugate against patient acute myeloid leukemia (AML) cells.

FIG. 3 provides two graphs showing the statistically significant (p<0.0001) distribution of Log IC₅₀ where CD33 antigens per cell are less than 5000 (bottom) versus greater than 5000 (top).

FIG. 4A is a graph showing the effect of IMGN779 on leukemic stem cells and normal hematopoietic stem cells.

FIG. 4B is a graph showing that IMGN779 spares normal hematopoietic stem cells.

FIG. 5A is a dot plot showing P-glycoprotein (PGP) activity as a function of CD33 expression in primary patient AML cells.

FIG. 5B is a dot plot showing IMGN779 cytotoxicity as a function of PGP activity in primary patient AML cells.

FIG. 6 is a table showing that AML cell lines are highly sensitive to IMGN779 and DGN462.

FIG. 7A is a graph showing the antitumor activity of IMGN779 against EOL-1 acute myeloid leukemia (AML) in subcutaneous xenografts in SCID mice after a single intravenous injection of IMGN779.

FIG. 7B is a graph showing the antitumor activity of IMGN779 against HL60/QC promyelocytic leukemia (PML) in subcutaneous xenografts in SCID mice after a single intravenous injection of IMGN779.

FIG. 7C is a table showing the effect of IMGN779 or a non-targeting antibody drug conjugate on human AML xenografts of EOL-1 and HL60/QC cells. “Treatment (T)/Control (C) (%)” refers to tumor growth inhibition ratio. “CR” refers to complete response.

FIG. 8 is a graph showing the percentage of mean body weight change over time in mice treated at 14 mg/kg and 40 mg/kg of IMGN779.

FIG. 9A is a graph showing plasma concentrations of total antibody and antibody conjugate over time.

FIG. 9B is a graph showing plasma concentrations of intact IMGN779 as measured by ELISA and biologically active concentration of IMGN779 as determined by a cytotoxicity assay.

FIG. 9C is a table showing in vivo stability and pharmacokinetics of IMGN779.

FIG. 10A provides the amino acid sequence of humanized My9-6 light chain.

FIG. 10B provides the amino acid sequence of humanized My9-6 heavy chain.

FIG. 11 is a graph showing in vitro IC₅₀ values for IMGN779 cytotoxicity and CD33 ABC (antibody binding capacity) in patient AML samples.

FIG. 12 is a graph showing in vitro IC₅₀ values for IMGN779 cytotoxicity in FLT3 WT and FLT3-ITD (Internal tandem duplication) patient AML samples.

FIG. 13 is a graph showing is a graph showing in vitro CD33 ABC in FLT3 WT and FLT3-ITD patient AML samples.

FIG. 14 is a table showing that IMGN779 has high cytotoxic activity in vitro against FLT3-ITD AML cell lines

FIG. 15 is a graph showing in vitro cytotoxicity of IMGN779 and FLT3 kinase inhibitors in the MOLM-13 AML cell line having the FLT3-ITD mutation.

FIG. 16 is a graph showing potent, antigen-targeted antitumor activity of IMGN779 against MV4-11 FLT3-ITD AML xenografts at a minimally efficacious dose of 10 μg/kg (DGN462 dose). T/C (%)=tumor growth inhibition; PR=partial tumor regression; CR=complete tumor regression.

FIG. 17 provides a mass spectroscopy analysis showing that there are approximately three DGN462 molecules conjugated per antibody (Drug to antibody ratio (DAR)).

### BRIEF DESCRIPTION OF THE SEQUENCES

Murine Heavy Chain CDR1:
(SEQ ID NO: 1)
SYYIH;
Murine Heavy Chain CDR2:
(SEQ ID NO: 2)
VIYPGNDDISYNQKFXG,
wherein X is K or Q;
Murine Heavy Chain CDR3:
(SEQ ID NO: 3)
EVRLRYFDV;
Murine Light Chain CDR1:
(SEQ ID NO: 4)
KSSQSVFFSSSQKNYLA;
Murine Light Chain CDR2:
(SEQ ID NO: 5)
WASTRES;
Murine Light Chain CDR3:
(SEQ ID NO: 6)
HQYLSSRT;
Murine Heavy Chain Variable Region:
(SEQ ID NO: 7)
QVQLQQPGAEVVKPGASVKMSCKASGYTFTSYYIHWIKQTPGQGLEW
VGVIYPGNDDISYNQKFKGKATLTADKSSTTAYMQLSSLTSEDSAVYY
CAREVRLRYFDVWGAGTTVTVSS;
Murine Light Chain Variable Region:
(SEQ ID NO: 8)
NIMLTQSPSSLAVSAGEKVTMSCKSSQSVFFSSSQKNYLAWYQQIPGQ
SPKLLIYWASTRESGVPDRFTGSGSGTDFTLTISSVQSEDLAIYYCHQY
LSSRTFGGGTKLEIKR;
Humanized Heavy Chain Variable Region:
(SEQ ID NO: 9)
QVQLQQPGAEVVKPGASVKMSCKASGYTFTSYYIHWIKQTPGQGLEW
VGVIYPGNDDISYNQKFQGKATLTADKSSTTAYMQLSSLTSEDSAVYY
CAREVRLRYFDVWGQGTTVTVSS;
Humanized Light Chain Variable Region:
(SEQ ID NO: 10)
EIVLTQSPGSLAVSPGERVTMSCKSSQSVFFSSSQKNYLAWYQQIPGQS
PRLLIYWASTRESGVPDRFTGSGSGTDFTLTISSVQPEDLAIYYCHQYLS
SRTFGQGTKLEIKR.

In particular embodiments, humanized antibodies include re-surfaced and/or CDR grafted antibodies.

### DETAILED DESCRIPTION OF THE INVENTION

The invention features compositions and methods that are useful for characterizing AML and selecting an efficacious therapy, as well methods for treating patients newly diagnosed with AML, patients experiencing AML relapse, and patients having refractory AML.

The invention is based, at least in part, on the discovery that a CD33-targeted antibody-drug conjugate (ADC) utilizing a novel DNA alkylator, DGN462, is highly active in vitro against primary patient AML cells and in vivo against AML xenografts in mice.

Despite high initial response rates of about 80% to chemotherapy, many acute myeloid leukemia (AML) patients experience a relapse of the disease. Without intending to be bound by theory, these relapses are thought to be due to the outgrowth of persistent leukemic stem cells. As reported herein below, the invention features a highly potent DNA alkylator, DGN462, which comprises an indolino-benzodiazepine dimer containing a mono-imine moiety.

IMGN779 is a CD33-targeted antibody drug conjugate comprising and anti-huCD33 antibody, also known as huMy9-6 or Z4681A, conjugated to a novel DNA-alkylating agent, DGN462, via a cleavable disulfide linker. Its favorable preclinical tolerability profile suggests that IMGN779 confers a therapeutic advantage over existing clinical agents for AML that demonstrate activity, but with significant toxicity. The highly potent, CD33-targeted activity of IMGN779 against AML cell lines and primary patient AML cells in vitro, the anti-tumor activity observed against AML xenografts in mice and the favorable safety profile support its advancement as a treatment for AML.

### Murine and Humanized My9-6 Antibody

Murine My9-6

A murine anti-CD33 antibody, variously designated herein as “My9-6”, “murine My9-6” and “muMy9-6”, is fully characterized with respect to the germline amino acid sequence of both light and heavy chain variable regions, amino acid sequences of both light and heavy chain variable regions, the identification of the CDRs, the identification of surface amino acids and means for its expression in recombinant form. See, for example, U.S. Pat. Nos. 7,557,189; 7,342,110; 8,119,787; 8,337,855 and U.S. Patent Publication No. 20120244171, each of which is incorporated herein by reference in their entirety.

The My9-6 antibody has also been functionally characterized and shown to bind with high affinity to CD33 on the surface of CD33-positive cells.

The term “variable region” is used herein to describe certain portions of antibody heavy chains and light chains that differ in sequence among antibodies and that cooperate in the binding and specificity of each particular antibody for its antigen. Variability is not usually evenly distributed throughout antibody variable regions. It is typically concentrated within three segments of a variable region called complementarity-determining regions (CDRs) or hypervariable regions, both in the light chain and the heavy chain variable regions. The more highly conserved portions of the variable regions are called the framework regions. The variable regions of heavy and light chains comprise four framework regions, largely adopting a beta-sheet configuration, with each framework region connected by the three CDRs, which form loops connecting the beta-sheet structure, and in some cases forming part of the beta-sheet structure. The CDRs in each chain are held in close proximity by the framework regions and, with the CDRs from the other chain, contribute to the formation of the antigen binding site of antibodies (E. A. Kabat et al. Sequences of Proteins of Immunological Interest, Fifth Edition, 1991, NIH).

The “constant” region is not involved directly in binding an antibody to an antigen, but exhibits various effector functions, such as participation of the antibody in antibody-dependent cellular toxicity.

Humanized My9-6 Antibody

Humanized versions of My9-6, variously designated herein as “huMy9-6”, and “humanized My9-6”, have also been prepared.

The goal of humanization is a reduction in the immunogenicity of a xenogenic antibody, such as a murine antibody, for introduction into a human, while maintaining the full antigen binding affinity and specificity of the antibody.

Humanized antibodies may be produced using several technologies, such as resurfacing and CDR grafting. As used herein, the resurfacing technology uses a combination of molecular modeling, statistical analysis and mutagenesis to alter the non-CDR surfaces of antibody variable regions to resemble the surfaces of known antibodies of the target host.

Strategies and methods for the resurfacing of antibodies, and other methods for reducing immunogenicity of antibodies within a different host, are disclosed in U.S. Pat. No. 5,639,641 (Pedersen et al.), which is hereby incorporated in its entirety by reference. Briefly, in a preferred method, (1) position alignments of a pool of antibody heavy and light chain variable regions are generated to give a set of heavy and light chain variable region framework surface exposed positions wherein the alignment positions for all variable regions are at least about 98% identical; (2) a set of heavy and light chain variable region framework surface exposed amino acid residues is defined for a rodent antibody (or fragment thereof); (3) a set of heavy and light chain variable region framework surface exposed amino acid residues that is most closely identical to the set of rodent surface exposed amino acid residues is identified; (4) the set of heavy and light chain variable region framework surface exposed amino acid residues defined in step (2) is substituted with the set of heavy and light chain variable region framework surface exposed amino acid residues identified in step (3), except for those amino acid residues that are within 5 angstroms of any atom of any residue of the complementarity-determining regions of the rodent antibody; and (5) the humanized rodent antibody having binding specificity is produced.

Antibodies can be humanized using a variety of other techniques including CDR-grafting (EP 0 239 400; WO 91/09967; U.S. Pat. Nos. 5,530,101; and 5,585,089), veneering or resurfacing (EP 0 592 106; EP 0 519 596; Padlan E. A., 1991, Molecular Immunology 28(4/5):489-498; Studnicka G. M. et al., 1994, Protein Engineering 7(6):805-814; Roguska M. A. et al., 1994, PNAS 91:969-973), and chain shuffling (U.S. Pat. No. 5,565,332). Human antibodies can be made by a variety of methods known in the art including phage display methods. See also U.S. Pat. Nos. 4,444,887, 4,716,111, 5,545,806, and 5,814,318; and international patent application publication numbers WO 98/46645, WO 98/50433, WO 98/24893, WO 98/16654, WO 96/34096, WO 96/33735, and WO 91/10741 (said references incorporated by reference in their entireties).

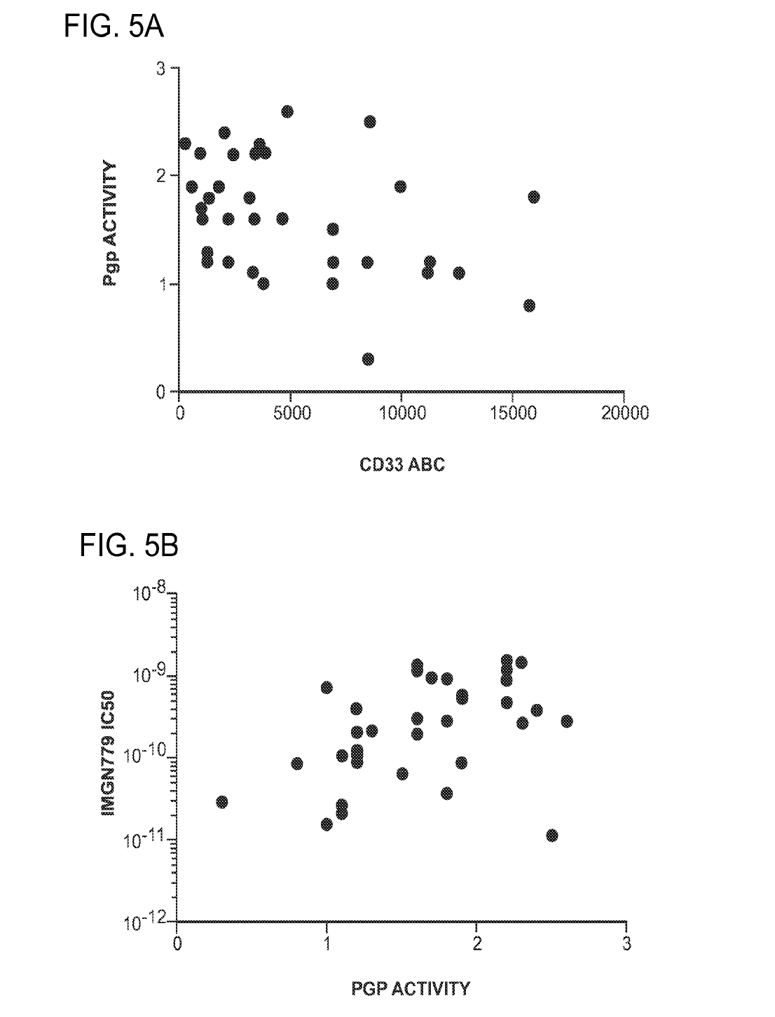

As further described herein, the CDRs of My9-6 were identified by modeling and their molecular structures were predicted. Humanized My9-6 antibodies were then prepared and have been fully characterized as described, for example in U.S. Patent Publication No. 20050118183, which is incorporated herein by reference. The amino acid sequences of the light and heavy chains of a number of huMy9-6 antibodies are shown in FIGS. 5A and 5B. See, for example, U.S. Pat. No. 8,337,855 and U.S. Patent Publication No. 20120244171, each of which is incorporated herein by reference.

### Epitope-Binding Fragments of the My9-6 Antibodies

Although epitope-binding fragments of the murine My9-6 antibody and the humanized My9-6 antibodies are discussed herein separately from the murine My9-6 antibody and the humanized versions thereof, it is understood that the term “antibody” or “antibodies” of the present invention may include both the full length muMy9-6 and huMy9-6 antibodies as well as epitope-binding fragments of these antibodies.

In a further embodiment, there are provided antibodies or epitope-binding fragments thereof comprising at least one complementarity-determining region having an amino acid sequence selected from the group consisting of SEQ ID NOs:1-6: SYYIH (SEQ ID NO:1), VIYPGNDDISYNQKFXG (SEQ ID NO:2), wherein X is K or Q, EVRLRYFDV (SEQ ID NO:3), KSSQSVFFSSSQKNYLA (SEQ ID NO:4), WASTRES (SEQ ID NO:5), HQYLSSRT (SEQ ID NO:6), and having the ability to bind CD33.

In a further embodiment, there are provided antibodies or epitope-binding fragments thereof comprising at least one heavy chain variable region and at least one light chain variable region, wherein said heavy chain variable region comprises three complementarity-determining regions having amino acid sequences represented by SEQ ID NOs:1-3, respectively, SYYIH (SEQ ID NO:1), VIYPGNDDISYNQKFXG (SEQ ID NO:2), wherein X is K or Q, EVRLRYFDV (SEQ ID NO:3), and wherein said light chain variable region comprises three complementarity-determining regions having amino acid sequences represented by SEQ ID NOs:4-6, respectively, KSSQSVFFSSSQKNYLA (SEQ ID NO:4), WASTRES (SEQ ID NO:5), HQYLSSRT (SEQ ID NO:6).

In a further embodiment, there are provided antibodies having a heavy chain variable region that has an amino acid sequence that shares at least 90% sequence identity with an amino acid sequence represented by SEQ ID NO:7: QVQLQQPGAEVVKPGASVKMSCKASGYTFTSYYIHWIKQTPGQGLEW VGVIYPGNDDISYNQKFKGKATLTADKSSTTAYMQLSSLTSEDSAVYY CAREVRLRYFDVWGAGTTVTVSS, more preferably 95% sequence identity with SEQ ID NO:7, most preferably 100% sequence identity with SEQ ID NO:7.

Similarly, there are provided antibodies having a light chain variable region that has an amino acid sequence that shares at least 90% sequence identity with an amino acid sequence represented by SEQ ID NO:8: NIMLTQSPSSLAVSAGEKVTMSCKSSQSVFFSSSQKNYLAWYQQIPGQ SPKLLIYWASTRESGVPDRFTGSGSGTDFTLTISSVQSEDLAIYYCHQY LSSRTFGGGTKLEIKR, more preferably 95% sequence identity with SEQ ID NO:8, most preferably 100% sequence identity with SEQ ID NO:8.

In a further embodiment, antibodies are provided having a humanized (e.g., resurfaced, CDR-grafted) heavy chain variable region that shares at least 90% sequence identity with an amino acid sequence represented by SEQ ID NO:9: QVQLQQPGAEVVKPGASVKMSCKASGYTFTSYYIHWIKQTPGQGLEW VGVIYPGNDDISYNQKFQGKATLTADKSSTTAYMQLSSLTSEDSAVYY CAREVRLRYFDVWGQGTTVTVSS, more preferably 95% sequence identity with SEQ ID NO:9, most preferably 100% sequence identity with SEQ ID NO:9.

Similarly, antibodies are provided having a humanized (e.g., resurfaced, CDR-grafted) light chain variable region that shares at least 90% sequence identity with an amino acid sequence corresponding to SEQ ID NO:10: EIVLTQSPGSLAVSPGERVTMSCKSSQSVFFSSSQKNYLAWYQQIPGQS PRLLIYWASTRESGVPDRFTGSGSGTDFTLTISSVQPEDLAIYYCHQYLS SRTFGQGTKLEIKR, more preferably 95% sequence identity with SEQ ID NO:10, most preferably 100% sequence identity with SEQ ID NO:10. In particular embodiments, the antibody includes conservative mutations in the framework region outside of the CDRs.

As used herein, “antibody fragments” include any portion of an antibody that retains the ability to bind to CD33, generally termed “epitope-binding fragments.” Examples of antibody fragments preferably include, but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Epitope-binding fragments, including single-chain antibodies, may comprise the variable region(s) alone or in combination with the entirety or a portion of the following: hinge region, C_{H1}, C_{H2}, and C_{H3} domains.

Such fragments may contain one or both Fab fragments or the F(ab′)₂ fragment. Preferably, the antibody fragments contain all six CDRs of the whole antibody, although fragments containing fewer than all of such regions, such as three, four or five CDRs, are also functional. Further, the functional equivalents may be or may combine members of any one of the following immunoglobulin classes: IgG, IgM, IgA, IgD, or IgE, and the subclasses thereof.

Fab and F(ab′)₂ fragments may be produced by proteolytic cleavage, using enzymes such as papain (Fab fragments) or pepsin (F(ab′)₂ fragments).

The single-chain FVs (scFvs) fragments are epitope-binding fragments that contain at least one fragment of an antibody heavy chain variable region (V_{H}) linked to at least one fragment of an antibody light chain variable region (V_{L}). The linker may be a short, flexible peptide selected to assure that the proper three-dimensional folding of the (V_{L}) and (V_{H}) regions occurs once they are linked so as to maintain the target molecule binding-specificity of the whole antibody from which the single-chain antibody fragment is derived. The carboxyl terminus of the (V_{L}) or (V_{H}) sequence may be covalently linked by a linker to the amino acid terminus of a complementary (V_{L}) and (V_{H}) sequence. Single-chain antibody fragments may be generated by molecular cloning, antibody phage display library or similar techniques well known to the skilled artisan. These proteins may be produced, for example, in eukaryotic cells or prokaryotic cells, including bacteria.

The epitope-binding fragments of the present invention can also be generated using various phage display methods known in the art. In phage display methods, functional antibody domains are displayed on the surface of phage particles which carry the polynucleotide sequences encoding them. In particular, such phage can be utilized to display epitope-binding domains expressed from a repertoire or combinatorial antibody library (e.g., human or murine). Phage expressing an epitope-binding domain that binds the antigen of interest can be selected or identified with antigen, e.g., using labeled CD33 or CD33 bound or captured to a solid surface or bead. Phage used in these methods are typically filamentous phage including fd and M13 binding domains expressed from phage with Fab, Fv or disulfide-stabilized Fv antibody domains recombinantly fused to either the phage gene III or gene VIII protein.

Examples of phage display methods that can be used to make the epitope-binding fragments of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

After phage selection, the regions of the phage encoding the fragments can be isolated and used to generate the epitope-binding fragments through expression in a chosen host, including mammalian cells, insect cells, plant cells, yeast, and bacteria, using recombinant DNA technology, e.g., as described in detail below. For example, techniques to recombinantly produce Fab, Fab′ and F(ab′)₂ fragments can also be employed using methods known in the art such as those disclosed in PCT publication WO 92/22324; Mullinax et al., 1992, BioTechniques 12(6):864-869; Sawai et al., 1995, AJRI34:26-34; and Better et al., 1988, Science 240:1041-1043; said references incorporated by reference in their entireties. Examples of techniques which can be used to produce single-chain Fvs and antibodies include those described in U.S. Pat. Nos. 4,946,778 and 5,258,498; Huston et al., 1991, Methods in Enzymology 203:46-88; Shu et al., 1993, PNAS 90:7995-7999; Skerra et al., 1988, Science 240:1038-1040.

### Functional Equivalents

Also included within the scope of the invention are functional equivalents of the My9-6 antibody and the humanized My9-6 antibodies. The term “functional equivalents” includes antibodies with homologous sequences, chimeric antibodies, modified antibody and artificial antibodies, for example, wherein each functional equivalent is defined by its ability to bind to CD33. The skilled artisan will understand that there is an overlap in the group of molecules termed “antibody fragments” and the group termed “functional equivalents.”

Antibodies with homologous sequences are those antibodies with amino acid sequences that have sequence identity or homology with amino acid sequence of the murine My9-6 and humanized My9-6 antibodies of the present invention. Preferably identity is with the amino acid sequence of the variable regions of the murine My9-6 and humanized My9-6 antibodies of the present invention. “Sequence identity” and “sequence homology” as applied to an amino acid sequence herein is defined as a sequence with at least about 90%, 91%, 92%, 93%, or 94% sequence identity, and more preferably at least about 95%, 96%, 97%, 98%, or 99% sequence identity to another amino acid sequence, as determined, for example, by the FASTA search method in accordance with Pearson and Lipman, Proc. Natl. Acad. Sci. USA 85, 2444-2448 (1988).

As used herein, a chimeric antibody is one in which different portions of an antibody are derived from different animal species. For example, an antibody having a variable region derived from a murine monoclonal antibody paired with a human immunoglobulin constant region. Methods for producing chimeric antibodies are known in the art. See, e.g., Morrison, 1985, Science 229:1202; Oi et al., 1986, BioTechniques 4:214; Gillies et al., 1989, J. Immunol. Methods 125:191-202; U.S. Pat. Nos. 5,807,715; 4,816,567; and 4,816,397, which are incorporated herein by reference in their entireties.

### Improved Antibodies

The CDRs are of primary importance for epitope recognition and antibody binding. However, changes may be made to the residues that comprise the CDRs without interfering with the ability of the antibody to recognize and bind its cognate epitope. For example, changes that do not affect epitope recognition, yet increase the binding affinity of the antibody for the epitope may be made.

Thus, also included in the scope of the present invention are improved versions of both the murine and humanized antibodies, which also specifically recognize and bind CD33, preferably with increased affinity.

Several studies have surveyed the effects of introducing one or more amino acid changes at various positions in the sequence of an antibody, based on the knowledge of the primary antibody sequence and on its properties such as binding and level of expression (Yang, W. P. et al., 1995, J. Mol. Biol., 254, 392-403; Rader, C. et al., 1998, Proc. Natl. Acad. Sci. USA, 95, 8910-8915; Vaughan, T. J. et al., 1998, Nature Biotechnology, 16, 535-539).

In these studies, equivalents of the primary antibody have been generated by changing the sequences of the heavy and light chain genes in the CDR1, CDR2, CDR3, or framework regions, using methods such as oligonucleotide-mediated site-directed mutagenesis, cassette mutagenesis, error-prone PCR, DNA shuffling, or mutator-strains of E. coli (Vaughan, T. J. et al., 1998, Nature Biotechnology, 16, 535-539; Adey, N. B. et al., 1996, Chapter 16, pp. 277-291, in “Phage Display of Peptides and Proteins”, Eds. Kay, B. K. et al., Academic Press). These methods of changing the sequence of the primary antibody have resulted in improved affinities of the secondary antibodies (Gram, H. et al., 1992, Proc. Natl. Acad. Sci. USA, 89, 3576-3580; Boder, E. T. et al., 2000, Proc. Natl. Acad. Sci. USA, 97, 10701-10705; Davies, J. and Riechmann, L., 1996, Immunotechnolgy, 2, 169-179; Thompson, J. et al., 1996, J. Mol. Biol., 256, 77-88; Short, M. K. et al., 2002, J. Biol. Chem., 277, 16365-16370; Furukawa, K. et al., 2001, J. Biol. Chem., 276, 27622-27628).

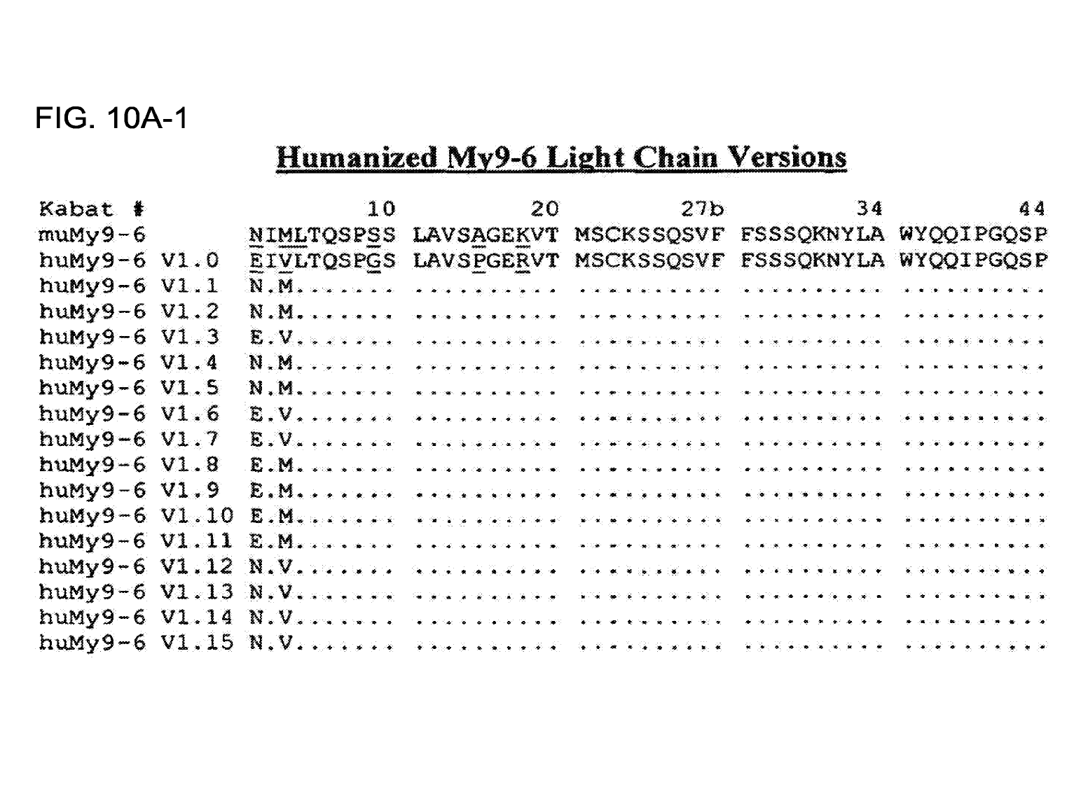

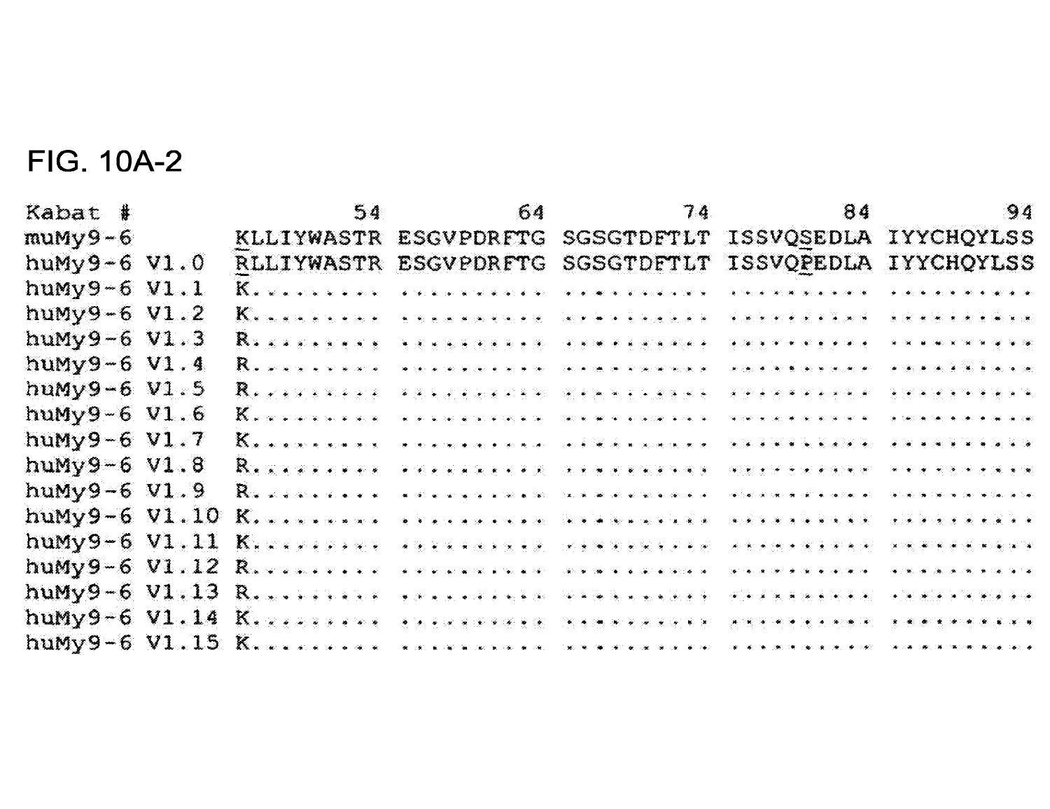

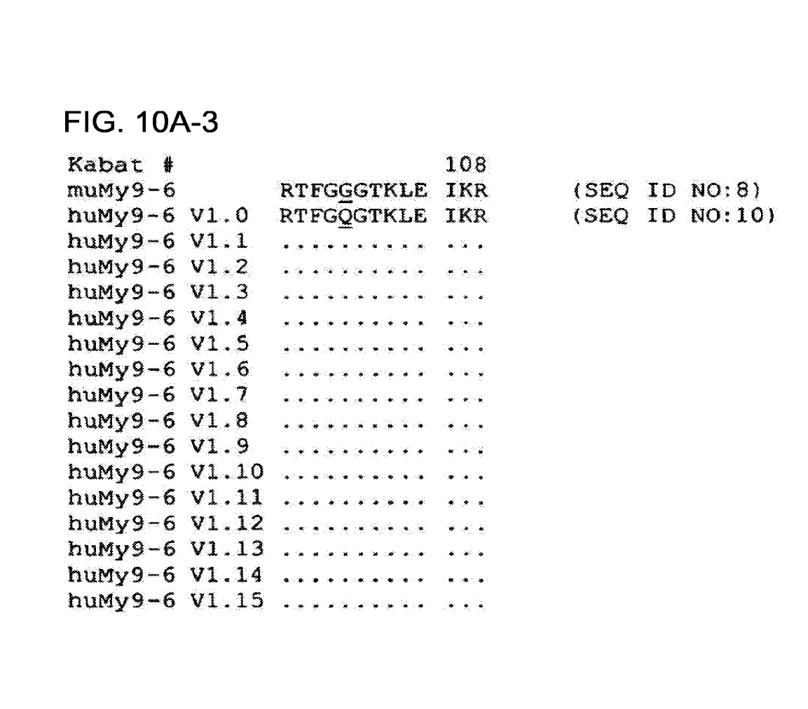

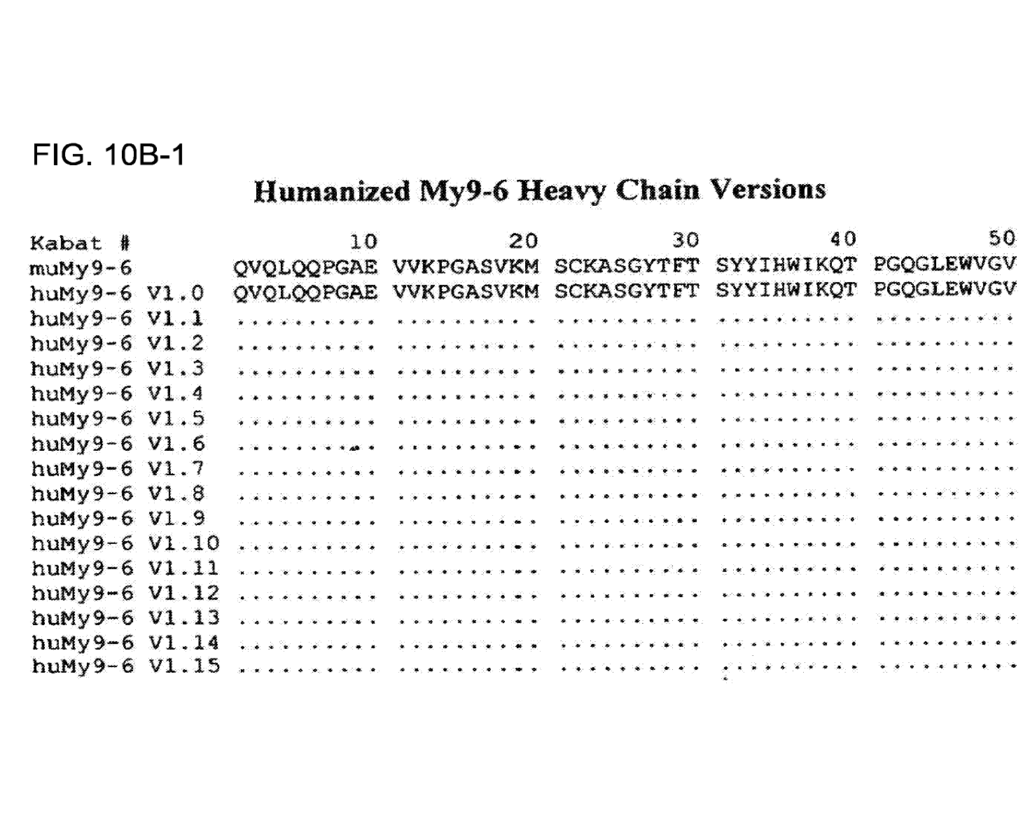

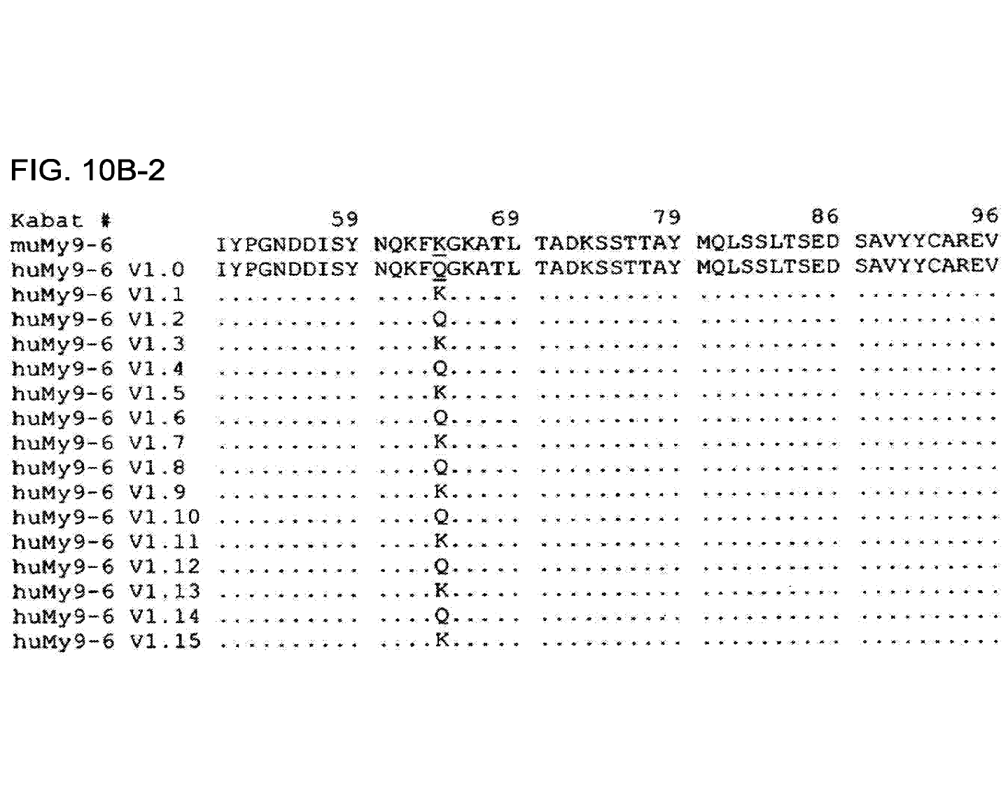

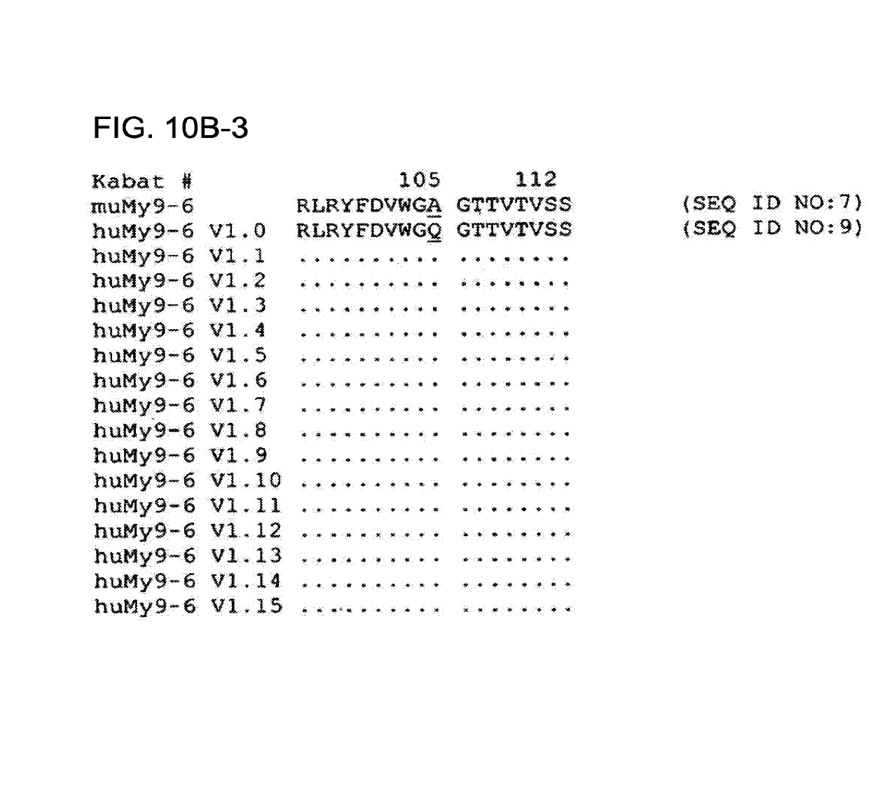

By a similar directed strategy of changing one or more amino acid residues of the antibody, the antibody sequences described herein (FIGS. 10A, 10B) can be used to develop anti-CD33 antibodies with improved functions, including improved affinity for CD33.

Improved antibodies also include those antibodies having improved characteristics that are prepared by the standard techniques of animal immunization, hybridoma formation and selection for antibodies with specific characteristics.

### Patient Stratification

The huMy9-6 antibody (also termed “Z4681A”) is useful for characterizing the expression of CD33 on cell derived from a subject, for example, during a biopsy. We have discovered that the level of CD33 expression on a cell of a subject is indicative of the efficacy of IMGN779 and, therefore, is useful for patient stratification. This discovery is based, at least in part, on patient data showing that primary patient cells expressing as little as 1,000 CD33 antigens per cell were highly sensitive to IMGN779 (60% of cells were sensitive). For samples with CD33 levels >3,000, more than 75% were highly sensitive to IMGN779. For samples with CD33 levels above 5,000, greater than 90% of cells were highly sensitive to IMGN779. Accordingly, patients having cells with at least about 1,000-25,000 CD33 antigens per cell (ABC, i.e., Antibody Binding Capacity) (e.g., 1,000, 1,500, 2,000, 2,500, 3,000, 3,500, 4,000, 4,500, 5,000, 5,500, 10,000, 15,000, 20,000, 25,000 ABC) or more are sensitive to treatment with IMGN779.

In particular embodiments, patients with ABC in the range of 1,000-18,000, 1,000-20,000, or 1,000-25,000; or in the range of 3,000-18,000, 3,000-20,000, or 3,000-25,000; or in the range of 5,000-18,000, 5,000-20,000, or 5,000-25,000 are selected and treated according to the methods of the invention. In particular embodiments, a patient is selected for treatment with IMGN779 where they are identified as having at least about 1,000, 2,000, 3,000, 4,000, 4,500, 5,000 or more antigens per cell. In other embodiments, a patient is selected and treated according to the methods of the invention where the lower limit of the ABC range is between about 1,000-5,000, between about 2,000-4,000, or between about 2,500-3,000, and the upper limit of the range is between about 18,000-25,000, 18,000-20,000, or 20,000-25,000. A comparison of IC₅₀ values for IMGN779 and another ADC that includes the same anti-CD33 antibody, but that includes a different linker and cytotoxin were 10,000 times higher even where the number of antigens per cell exceeded 5,000.

In still other embodiments, a newly diagnosed patient is selected for therapy when they are identified as having at least about 5,000, 6,000, or 7,000 antigens per cell or more. A relapsed AML patient is selected for IMGN779 therapy when they are identified as having at least about 1,000, 2,000, 2,500, 3,000, 3,500, 4,000, 4,500, 5,000 antigens per cell or more. An AML patient with refractory disease is selected for IMGN779 therapy when they are identified as having at least about 1,000, 2,000, 2,500, 3,000, 3,500, 4,000, 4,500, 5,000 antigens per cell or more. In particular embodiments, a relapsed or refractory AML patient with an ABC in the range of 1,000-18,000, 1,000-20,000, or 1,000-25,000; or in the range of 3,000-18,000, 3,000-20,000, or 3,000-25,000; or in the range of 5,000-18,000, 5,000-20,000, or 5,000-25,000 is selected and treated according to the methods of the invention. In particular embodiments, a relapsed or refractory AML patient is selected for treatment with IMGN779 where they are identified as having at least about 1,000, 2,000, 3,000, 4,000, 4,500, 5,000 or more antigens per cell. In other embodiments, a relapsed or refractory AML patient is selected and treated according to the methods of the invention where the lower limit of the ABC range is between about 1,000-5,000, between about 2,000-4,000, or between about 2,500-3,000, and the upper limit of the range is between about 18,000-25,000, 18,000-20,000, or 20,000-25,000.

Therefore, it was entirely unexpected that IMGN779 would be effective at such low CD33 values and at such low concentrations. CD33 expression on primary patient AML cells was measured using a calibrated flow cytometry method. AML samples were stained with a phycoerythrin (PE)-conjugated anti-CD33 antibody (clone WM53, BD Biosciences) and compared with the fluorescent signal of a calibration curve using Quantibrite beads (PE conjugated-beads at varying label to bead ratio), allowing the total number of CD33 antibodies bound per AML cell (ABC value) to be determined CD33 is expressed at relatively low levels in patient AML cells, with maximal expression of approximately 17,000 ABC.

### Use of IMGN779 for the Treatment of AML and Minimal Residual Disease

Although many AML patients respond to chemotherapy, large numbers of these patients eventually relapse. AML relapse is thought to be associated with the persistence of some number of leukemic stem cells. The present invention provides methods for treating AML that involve specifically targeting leukemic stem cells. Accordingly, the invention provides methods for achieving complete remission in an AML patient. Advantageously, IMGN779 specifically targets leukemic stem cells, while normal hematopoietic stem cells are spared. IMGN is therefore predicted to have an improved toxicity profile relative to other chemotherapeutics, which do not spare normal hematopoietic stem cells.

Given the specificity of IMGN779 for leukemic stem cells, IMGN779 will likely benefit patients experiencing relapse by reducing the likelihood of minimal residual disease. IMGN779 is not limited to the treatment of relapse, however. It is expected to be superior to conventional chemotherapeutic agents because it targets not only CD33 expressing blasts, but also leukemic stem cells, which are likely responsible for relapse. Accordingly, the invention provides methods for treating AML in patients that are newly diagnosed, relapsed, and refractory.

### Conjugates

IMGN779 is an antibody drug conjugate comprising DGN462 conjugated to the anti-huCD33 antibody, Z4681A, via a cleavable disulfide linker. DGN462 comprises an indolino-benzodiazepine dimer containing a mono-imine moiety.

In one embodiment, a conjugate of the present invention comprises the monoclonal antibody described herein (e.g., huMy9-6, also termed “Z4681A”) coupled to a cytotoxic benzodiazepine dimer compound represented by the following structural formula:

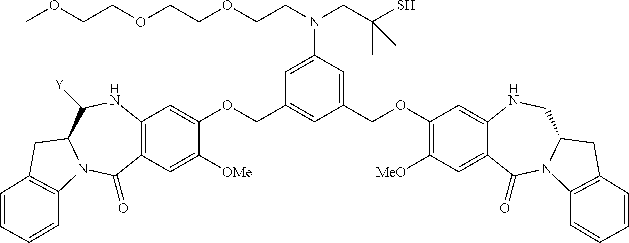

via N-succinimidyl-4-(2-pyridyldithio)-2-sulfobutanoate (sulfo-SPDB) linker or a pharmaceutically acceptable salt thereof, wherein Y is —SO₃M and M is H or a pharmaceutically acceptable cation. In one embodiment, M is Na⁺ or K⁺. In another embodiment, M is Na⁺. In yet another embodiment, M is H.

The sulfo-SPDB linker is known in the art and is described in U.S. Pat. No. 8,236,319. The sulfo-SPDB linker can be represented by the following structural formula:

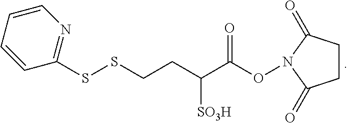 [SMILES: O=C(ON1C(=O)CCC1=O)C(CCSSc1ccccn1)S(=O)(=O)O]

In another embodiment, the conjugate of the present invention comprises the monoclonal antibody described herein (e.g., huMy9-6) coupled to a cytotoxic benzodiazepine dimer compound represented by the following structural formula:

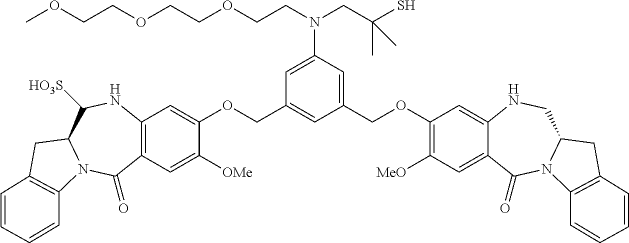

via the sulfo-SPDB linker or a pharmaceutically acceptable salt thereof.

In another embodiment, the conjugate of the present invention comprises the monoclonal antibody described herein (e.g., huMy9-6) coupled to a cytotoxic benzodiazepine dimer compound represented by the following structural formula:

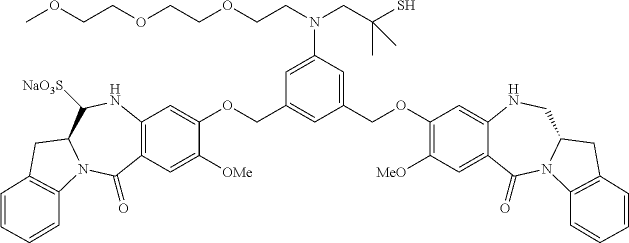

via the sulfo-SPDB linker or a pharmaceutically acceptable salt thereof.

In another embodiment, the conjugate of the present invention comprises the monoclonal antibody described herein (e.g., huMy9-6) coupled to a cytotoxic benzodiazepine dimer compound represented by the following structural formula:

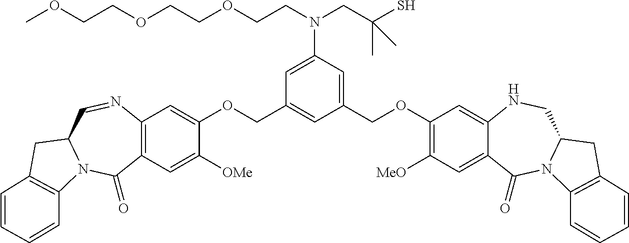 [SMILES: COCCOCCOCCN(CC(C)(C)S)c1cc(COc2cc3c(cc2OC)C(=O)N2c4ccccc4C[C@H]2C=N3)cc(COc2cc3c(cc2OC)C(=O)N2c4ccccc4C[C@H]2CN3)c1]

via the sulfo-SPDB linker or a pharmaceutically acceptable salt thereof.

In yet another embodiment, the conjugate of the present invention are represented by the following structural formula:

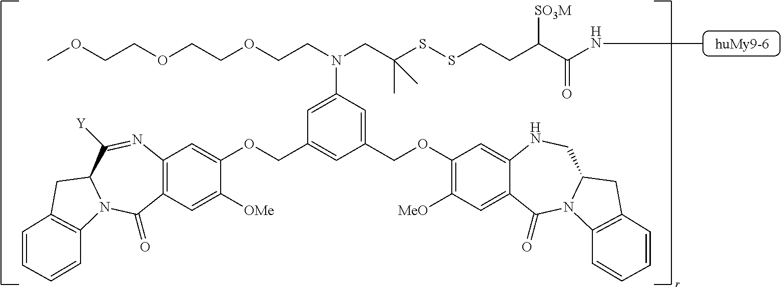

or a pharmaceutically acceptable salt thereof, wherein r is an integer from 1 to 10, Y is —SO₃M and M, for each occurrence, is independently —H or a pharmaceutically acceptable cation. In one embodiment, M is Na⁺ or K⁺. In another embodiment, M is Na⁺.

In another embodiment, the conjugate of the present invention is represented by the following structural formula:

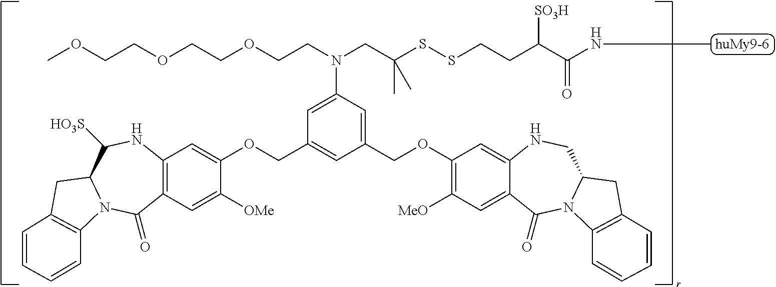

or a pharmaceutically acceptable salt thereof, wherein r is an integer from 1 to 10.

In another embodiment, the conjugate of the present invention is represented by the following structural formula:

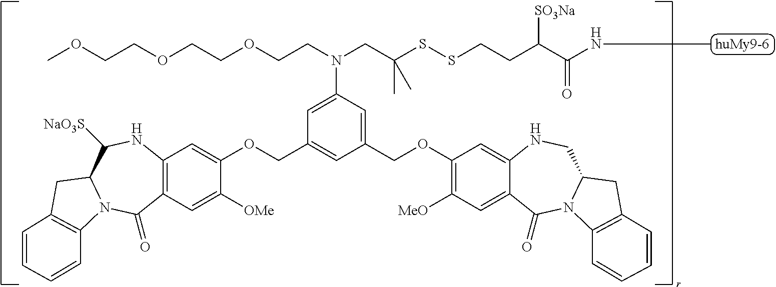

or a pharmaceutically acceptable salt thereof, wherein r is an integer from 1 to 10.

In another embodiment, the conjugate of the present invention is represented by the following structural formula:

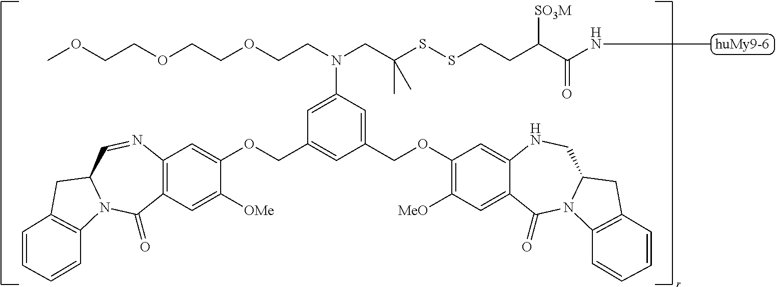

or a pharmaceutically acceptable salt thereof, wherein r is an integer from 1 to 10, and M is —H or a pharmaceutically acceptable cation. In one embodiment, M is Na⁺ or K⁺. In another embodiment, M is Na⁺. In yet another embodiment, M is H.

In another embodiment, the conjugate of the present invention is represented by the following structural formula:

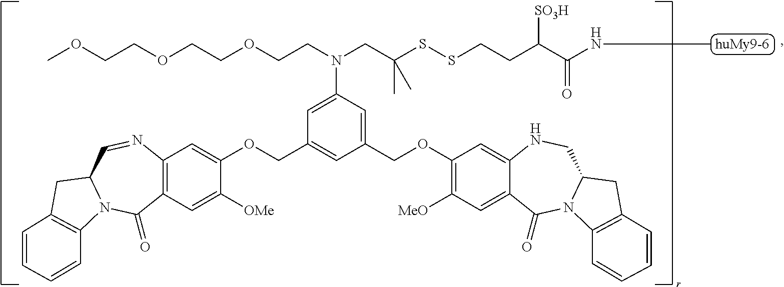

or a pharmaceutically acceptable salt thereof.

In another embodiment, the conjugate of the present invention is represented by the following structural formula:

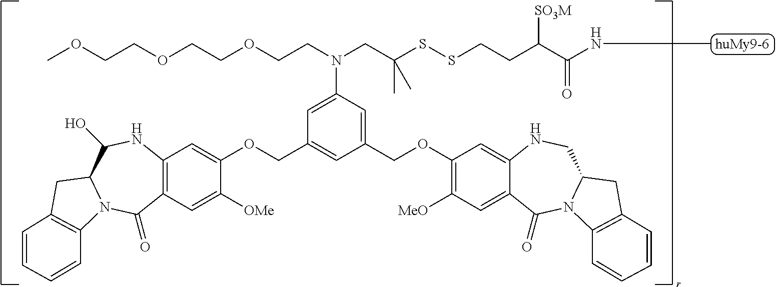

or a pharmaceutically acceptable salt thereof.

In another embodiment, the conjugate of the present invention comprises the monoclonal antibody described herein (e.g., huMy9-6) coupled to a cytotoxic benzodiazepine dimer compound represented by the following structural formula:

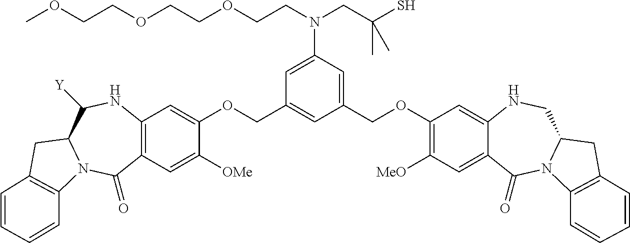

via N-succinimidyl-4-(2-pyridyldithio)butanoate (SPDB) linker, wherein Y is —SO₃M and M is H or a pharmaceutically acceptable cation. In one embodiment, M is Na⁺ or K⁺. In another embodiment, M is Na⁺.

The SPDB linker is known in the art and is describe in U.S. Pat. No. 6,913,748. The SPDB linker can be represented by the following structural formula:

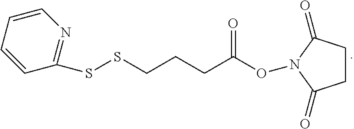 [SMILES: O=C(CCCSSc1ccccn1)ON1C(=O)CCC1=O]

In another embodiment, the conjugate of the present invention comprises the monoclonal antibody described herein (e.g., huMy9-6) coupled to a cytotoxic benzodiazepine dimer compound represented by the following structural formula:

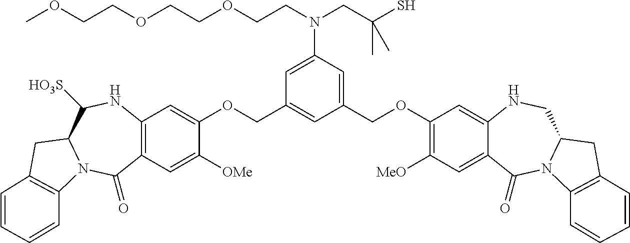

via the SPDB linker.

In another embodiment, the conjugate of the present invention comprises the monoclonal antibody described herein (e.g., huMy9-6) coupled to a cytotoxic benzodiazepine dimer compound represented by the following structural formula:

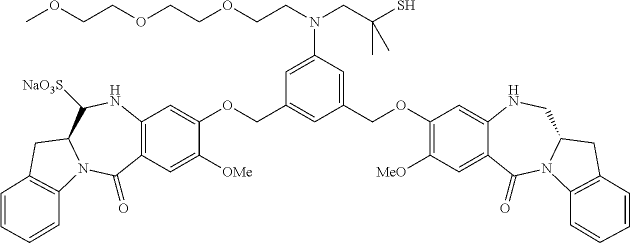

via the SPDB linker.

In another embodiment, the conjugate of the present invention comprises the monoclonal antibody described herein (e.g., huMy9-6) coupled to a cytotoxic benzodiazepine dimer compound represented by the following structural formula:

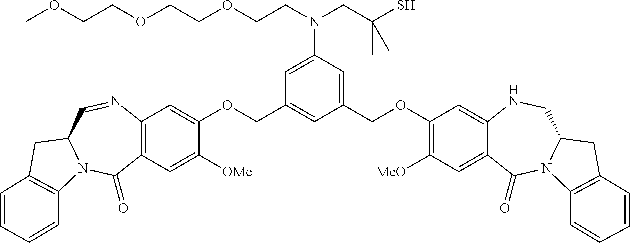 [SMILES: COCCOCCOCCN(CC(C)(C)S)c1cc(COc2cc3c(cc2OC)C(=O)N2c4ccccc4C[C@H]2C=N3)cc(COc2cc3c(cc2OC)C(=O)N2c4ccccc4C[C@H]2CN3)c1]

via SPDB linker.

In yet another embodiment, the conjugate of the present invention is represented by the following structural formula:

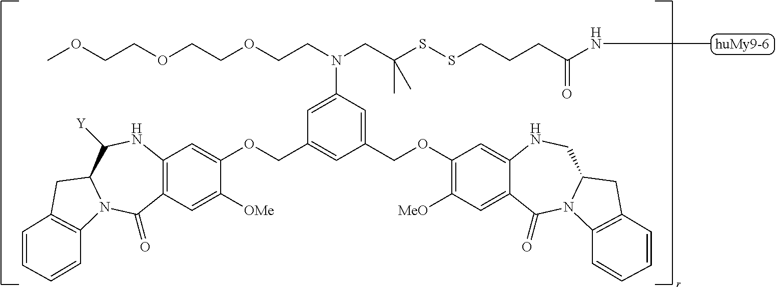

or a pharmaceutically acceptable salt thereof, wherein r is an integer from 1 to 10, Y is —SO₃M and M, for each occurrence, is independently —H or a pharmaceutically acceptable cation. In one embodiment, M is Na⁺ or K⁺.

In another embodiment, the conjugate of the present invention is represented by the following structural formula:

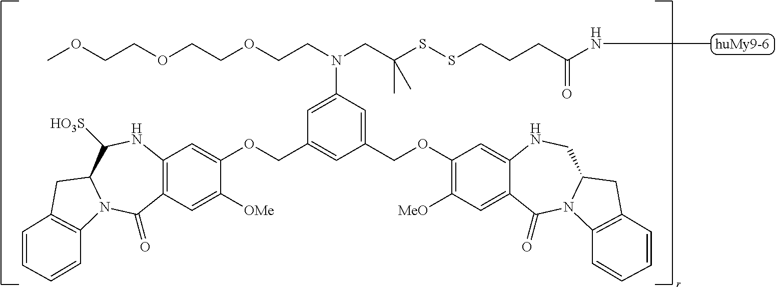

or a pharmaceutically acceptable salt thereof, wherein r is an integer from 1 to 10.

In another embodiment, the conjugate of the present invention is represented by the following structural formula:

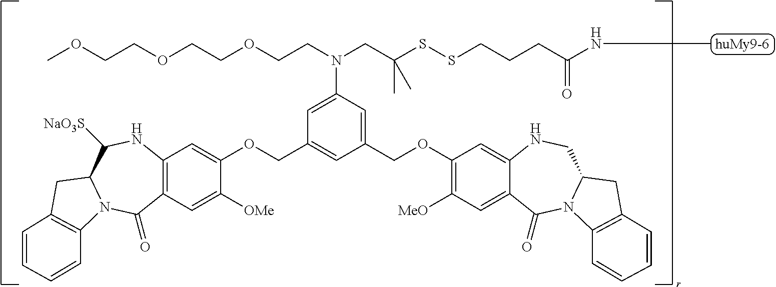

or a pharmaceutically acceptable salt thereof, wherein r is an integer from 1 to 10.

In another embodiment, the conjugate of the present invention is represented by the following structural formula:

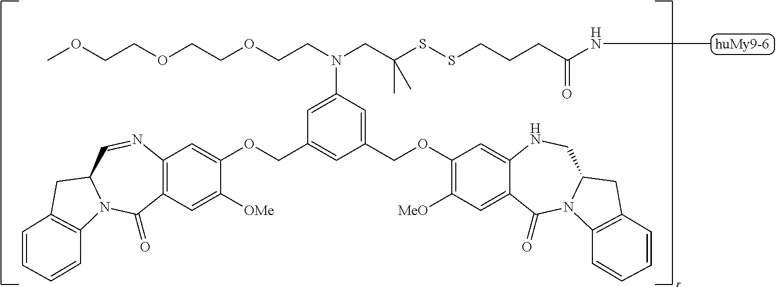

or a pharmaceutically acceptable salt thereof, wherein r is an integer from 1 to 10.

In yet another embodiment, the conjugate of the present invention is represented by the following structural formula:

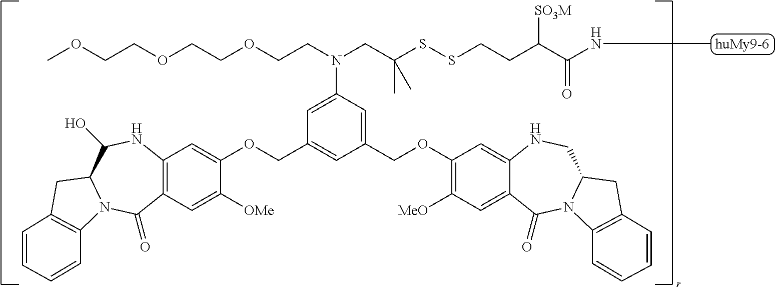

or a pharmaceutically acceptable salt thereof, wherein r is an integer from 1 to 10.

In certain embodiments, the conjugate described herein may comprise 1-10 cytotoxic benzodiazepine dimer compounds, 2-9 cytototoxic benzodiazepine dimer compounds, 3-8 cytotoxic benzodiazepine dimer compounds, 4-7 cytotoxic benzodiazepine dimer compounds, or 5-6 cytotoxic benzodiazepine dimer compounds.

In certain embodiments, a composition comprising the conjugates described herein may comprise an average 1-10 cytotoxic benzodiazepine dimer molecule per antibody molecule. The average ratio of cytotoxic benzodiazepine dimer molecule per antibody molecule is referred to herein as the Drug Antibody Ratio (DAR). In one embodiment, the DAR is between 2-8, 3-7, 3-5 or 2.5-3.5.

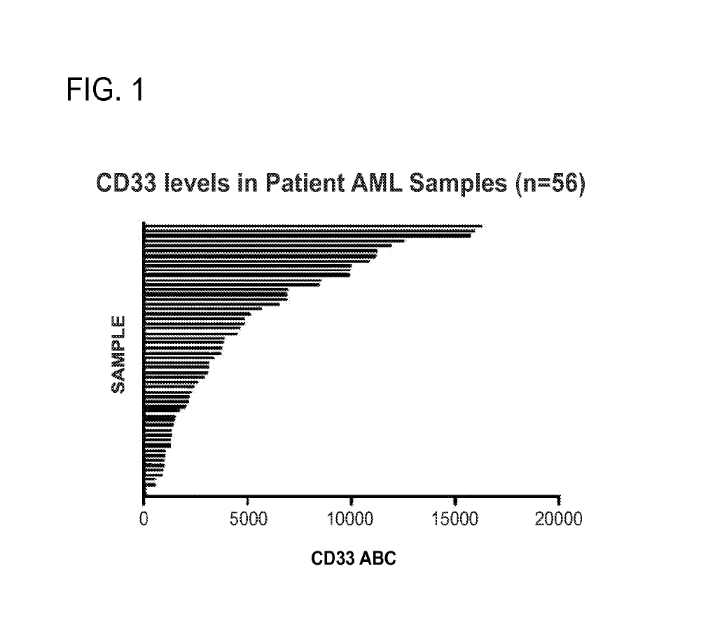

The cytotoxic benzodiazepine dimer compound and the conjugates described herein can be prepared according to methods described in US 2012/0244171 and US 2012/0238731, for example, but limited to, paragraphs [0395]-[0397] and [0598]-[0607], FIGS. 1, 15, 22, 23, 38-41, 43, 48, 55 and 60, and Examples 1, 6, 12, 13, 20, 21, 22, 23, 26-30 and 32 of US2012/0244171 and paragraphs [0007]-[0105], [0197]-[0291], FIGS. 1-11, 16, 28 and Examples 1-7, 9-13, 15 and 16 of US 2012/0238731.

The term “cation” refers to an ion with positive charge. The cation can be monovalent (e.g., Na⁺, K⁺, etc.), bi-valent (e.g., Ca²⁺, Mg²⁺, etc.) or multi-valent (e.g., Al³⁺ etc.). Preferably, the cation is monovalent.

The phrase “pharmaceutically acceptable” indicates that the substance or composition must be compatible chemically and/or toxicologically, with the other ingredients comprising a formulation, and/or the mammal being treated therewith.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

If the compound of the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the compound of the invention is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

### IMGN779

In one embodiment, IMGN779 may be represented as the bis-acid depicted below or any pharmaceutically acceptable salt thereof

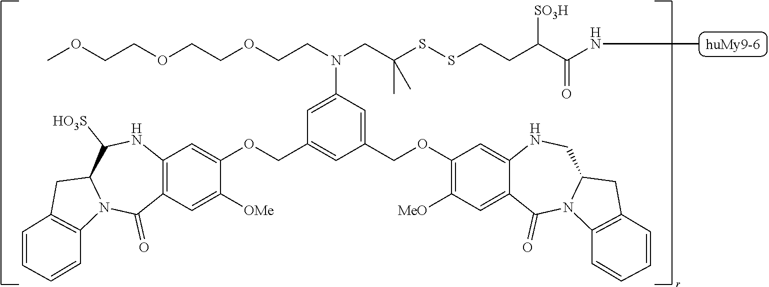

In other embodiments, IMGN779 may be represented as the active ingredient or any pharmaceutically acceptable salt thereof:

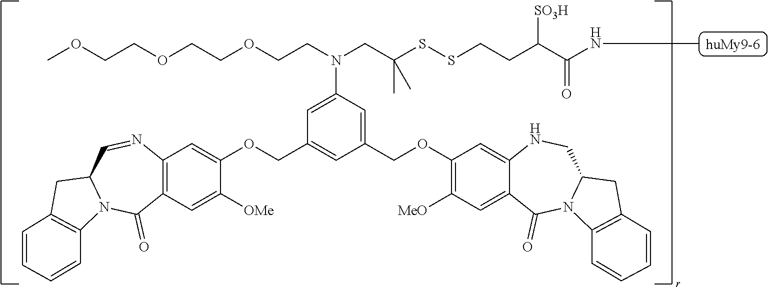

In other embodiments, the following formula may be used, which encompasses the bis acid, as well as salts:

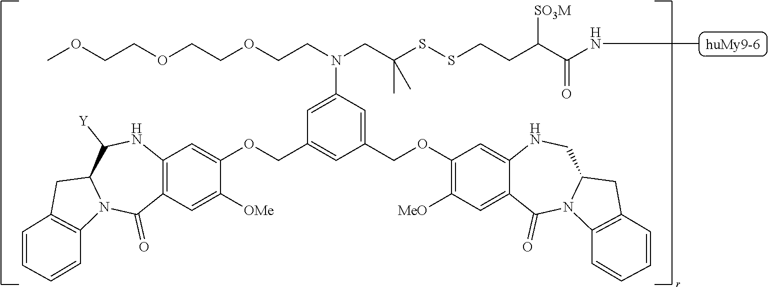

wherein r is an integer from 1 to 10, Y is —H or —SO₃M, preferably where Y is —SO₃M, and M is —H or a pharmaceutically acceptable cation.

In other embodiments, the following formula may be used, which encompasses the bis acid, as well as salts:

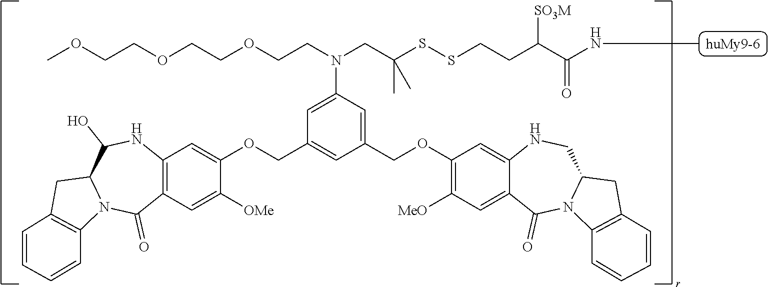

### P-glycoprotein

P-glycoprotein (PGP), also known as MDR1, is an ATP-dependent drug efflux pump of 170 kD. It is a member of the ABC superfamily and is abundantly expressed in multidrug resistance (MDR) cells and produced by the ABCB1 gene. AML cells expressing PGP are, at least to some degree, resistant to treatment with conventional chemotherapeutics. Importantly, the invention advantageously provides for the treatment of multi-drug resistant AML. In particular embodiments, the invention provides methods for treating PGP-expressing AML.

### Therapeutic Applications

The present invention provides methods of administering IMGN779 for the treatment of AML, including multi-drug resistant AML. In particular embodiments, IMGN779 is administered to a subject in a pharmaceutically acceptable dosage form. IMGN779 may be administered intravenously as a bolus or by continuous infusion over a period of time, by intramuscular, subcutaneous, intra-articular, intrasynovial, intrathecal, oral, topical, or inhalation routes. Pharmaceutical compositions comprising IMGN779 is administered by intratumoral, peritumoral, intralesional, or perilesional routes, to exert local as well as systemic therapeutic effects.

A pharmaceutically acceptable dosage form will generally include a pharmaceutically acceptable agent such as a carrier, diluent, and excipient. These agents are well known and the most appropriate agent can be determined by those of skill in the art as the clinical situation warrants. Examples of suitable carriers, diluents and/or excipients include: (1) Dulbecco's phosphate buffered saline, pH .about.7.4, containing about 1 mg/ml to 25 mg/ml human serum albumin, (2) 0.9% saline (0.9% w/v NaCl), and (3) 5% (w/v) dextrose.

When present in an aqueous dosage form, rather than being lyophilized, IMGN779 typically will be formulated at a concentration of about 0.1 mg/ml to 100 mg/ml, although wide variation outside of these ranges is permitted. For the treatment of disease, the appropriate dosage of IMGN779 will depend on the type of disease to be treated, as defined above, the severity and course of the disease, whether the antibodies are administered for preventive or therapeutic purposes, the course of previous therapy, the patient's clinical history and response to the antibody, and the discretion of the attending physician. The antibody is suitably administered to the patient at one time or over a series of treatments.

The therapeutic applications of the present invention include methods of treating a subject having a disease. The diseases treated with the methods of the present invention are those characterized by the expression of CD33. Such diseases include myelodysplastic syndromes (MDS) and cancers such as acute myeloid leukemia (AML), chronic myeloid leukemia (CML) and acute pro-myelocytic leukemia (APL). The skilled artisan will understand that the methods of the present invention may also be used to treat other diseases yet to be described but characterized by the expression of CD33.

The therapeutic applications of the present invention can be also practiced in vitro and ex vivo.

### Polynucleotides, Vectors, Host Cells and Methods for Making Antibody

The present invention further provides polynucleotides comprising a nucleotide sequence encoding an antibody of the invention or epitope-binding fragments thereof.

The polynucleotides may be obtained, and the nucleotide sequence of the polynucleotides determined, by any method known in the art. For example, if the nucleotide sequence of the antibody is known, a polynucleotide encoding the antibody may be assembled from chemically synthesized oligonucleotides (e.g., as described in Kutmeier et al., 1994, BioTechniques 17:242) which, briefly, involves the synthesis of overlapping oligonucleotides containing portions of the sequence encoding the antibody, annealing and ligation of those oligonucleotides, and then amplification of the ligated oligonucleotides by PCR.

Methods for the construction of recombinant vectors containing antibody coding sequences and appropriate transcriptional and translational control signals are well known in the art. These methods include, for example, in vitro recombinant DNA techniques, synthetic techniques, and in vivo genetic recombination. The invention, thus, provides replicable vectors comprising a nucleotide sequence encoding an antibody molecule of the present invention, or a heavy or light chain thereof, or a heavy or light chain variable domain, or an epitope-binding fragment of any of these, operably linked to a promoter.

The recombinant vector is transferred to a host cell by conventional techniques and the transfected cells are then cultured by conventional techniques to produce an antibody of the invention. Thus, the invention includes host cells containing a polynucleotide encoding an antibody of the invention, or an epitope-binding fragment thereof, operably linked to a heterologous promoter. In preferred embodiments, vectors encoding both the heavy and light chains may be co-expressed in the host cell for expression of an entire immunoglobulin molecule.

A variety of host-expression vector systems may be utilized to express the antibody molecules of the invention. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; Cockett et al., 1990, Bio/Technology 8:2).

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.) and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compounds that interact directly or indirectly with the antibody molecule.

Once an antibody molecule of the invention has been recombinantly expressed, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins.

### Kits

The invention provides kits comprising an anti-CD33 antibody (e.g., clone WM53, BD Biosciences) that detects the level of CD33 expression in a patient sample (e.g., the number of antigens per cell) and a therapeutic composition comprising an effective amount of IMGN779. If desired, the kit further comprises directions for detecting the level of CD33 expression and determining whether or not IMGN779 would be effective if administered to the patient. Optionally, the kit further comprises instructions for administering IMGN779 to a patient selected to receive IMGN779.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### IMGN779 Exhibited CD33-Specific In Vitro Cytotoxicity Against Primary Patient AML Cells

CD33 levels and P-glycoprotein (Pgp) activity were measured by flow cytometry. Cytotoxic potencies of DGN462 and IMGN779 in AML cell lines were evaluated using continuous exposure up to 7 days, with WST-8 viability staining Potency of IMGN779 against primary AML samples and normal bone marrow (NBM) was evaluated using colony formation assays after 24-hour exposure and after long term liquid culture to assess the potency in leukemic progenitors and leukemic stem cells, respectively. The antitumor activity of IMGN779 was assessed in SCID mice bearing subcutaneous HL60/QC and EOL-1 xenografts.

Pharmacokinetic parameters in CD-1 mice were determined from plasma concentrations of IMGN779 conjugate and its total Z4681A antibody component at various time points, measured by ELISA. The bioactivity of a subset of these plasma samples was confirmed by assay of cytotoxic potency against AML cells. The tolerability of IMGN779 was evaluated in CD-1 mice, with measurements of body weight, clinical observations and clinical chemistries.

IMGN779 demonstrated highly potent and CD33-specific in vitro cytotoxicity against primary patient AML cells isolated from peripheral blood or bone marrow samples. IC₅₀ values ranged from 10 to 1500 pM with the highest activity generally observed in samples with CD33 expression levels >3000 or 5000 antigens per cell. In long term cultures, IMGN779 showed a dose dependent decrease of leukemic colony formation in patient AML samples. In contrast, colony formation increased in normal bone marrow, indicating that normal hematopoietic stem cells were spared.

PGP activity inversely correlated with CD33-expression levels and IMGN779 cytotoxicity. IMGN779 was highly active against AML cell lines, including PGP-expressing cell lines, with IC₅₀ values ranging from 2 to 3000 pM. IMGN779 was highly active against AML xenografts, with a minimal efficacious dose (MED) of 0.6 mg/kg (conjugate dose). Conjugate half-life was approximately 3-4 days in mice, with bioactivity maintained for at least 3 days, indicating that the conjugate remains intact and active during circulation. IMGN779 had favorable tolerability in mice (maximum tolerated dose of 40 mg/kg) without delayed toxicity or liver toxicity.

### Example 2

### CD33 is Expressed on Primary Patient AML Cells

CD33 expression on primary patient AML cells was measured using a calibrated flow cytometry method (FIG. 1). AML samples were stained with a fluorescent-tagged anti-CD33 antibody and compared with the fluorescent signal of a calibration curve using fluorescent-tagged beads at varying label to bead ratio, allowing the total number of CD33 antibodies bound per AML cell (ABC value) to be determined CD33 is expressed at relatively low levels in patient AML cells, with maximal expression of approximately 17,000 antigens per cell (ABC).

### Example 3

### IMGN779 Demonstrates Highly Potent and CD33 Specific In Vitro Cytotoxicity Against Primary Patient AML Cells

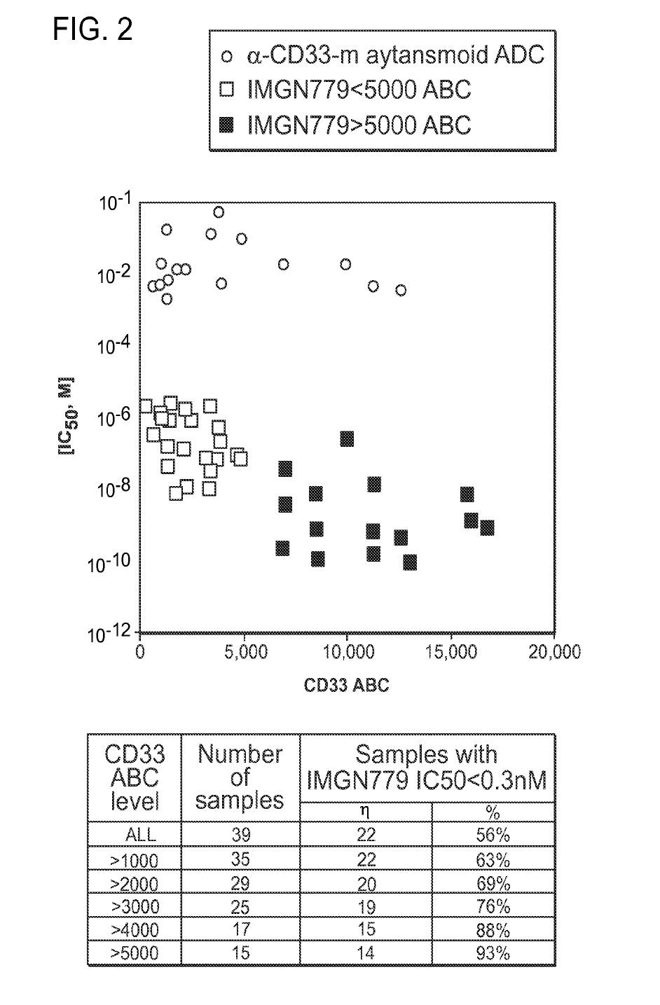

The cytotoxic activity of IMGN779 was assessed against a panel of primary patient AML cells in colony-forming assays after 24 hour conjugate exposure (FIG. 2).

The activity of a CD33-targeting maytansinoid ADC (using the same antibody in IMGN779) was assessed on a subset of these samples. IMGN779 was highly active against patient AML cells with IC₅₀ values ranging from 11 pM to 1.6 nM, with a dependence on CD33 expression level. CD33 levels ranged from ˜200 to 16,000 antigens per cell. In contrast, the CD33-targeting maytansinoid ADC was between 60 to 9,000-fold less active than IMGN779, with no dependence on CD33 expression level. FIG. 2 shows the in vitro potency of IMGN779 compared with a CD33-targeting maytansinoid ADC against patient AML cells.

Using an IC₅₀ cutoff of 0.3 nM to define a high level of sensitivity (500-fold lower than the median IC₅₀ of the CD33-targeting maytansinoid ADC), the percent of patient cells highly sensitive to IMGN779 based on CD33 expression cutoff was determined. For samples with CD33 levels greater than 1000, more than 60% were highly sensitive. For samples with CD33 levels >3,000, more than 75% were highly sensitive to IMGN779. For samples with CD33 levels above 5,000, greater than 90% of cells were highly sensitive to IMGN779, although sample numbers were lower (14 of 15 samples). When all samples, independent of CD33 level, were included, only 56% of all samples were highly sensitive. The percent of highly sensitive samples increased with CD33 level. Patient AML cells expressing CD33 levels above 5,000 ABC were significantly more sensitive (comparison of median IC₅₀ values) than those with less than 5,000 CD33 antigens per cell (p<0.0001).

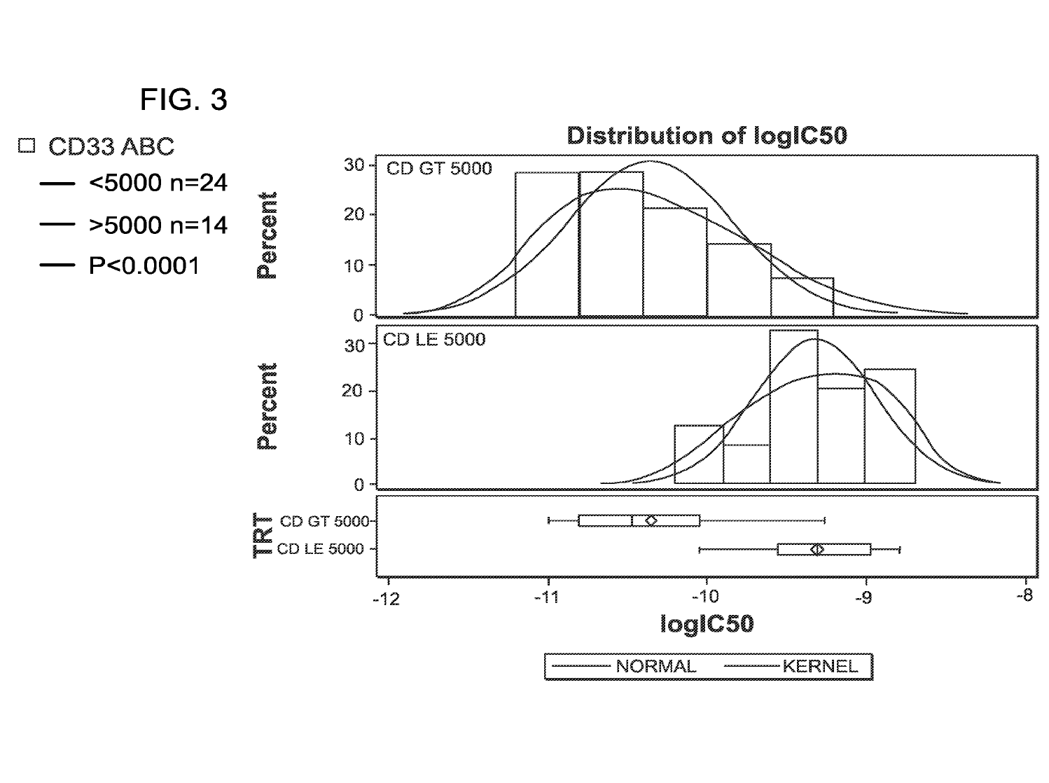

The cytotoxic activity of a non-binding chimeric IgG1-DGN462 conjugate was also assessed to determine the CD33-dependence of the observed IMGN779 activity. The non-CD33 binding conjugate was generally inactive against these cells, with IC₅₀ values not reached in most samples at the highest dose tested (1 nM), demonstrating that the highly potent IMGN779 activity is dependent upon CD33 targeting. Distribution of log IC₅₀ in cells where CD33 antigens per cell are less than 5000 or greater than 5000 are shown at FIG. 3.

### Example 4

### IMGN779 Specifically Targeted Leukemic Stem Cells, while Sparing Normal Hematopoietic Stem Cells

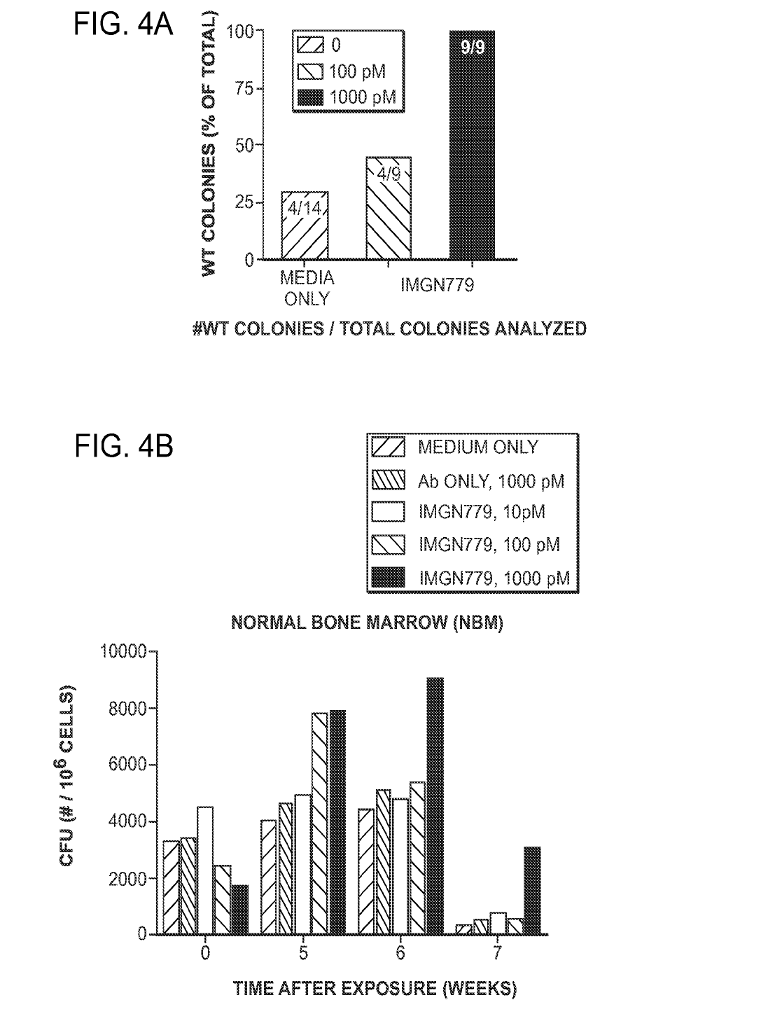

Colonies formed in a colony forming unit (CFU) assay after exposure of AML cells to IMGN779 were collected after long-term liquid culture (5-7 weeks) and analyzed for FLT3-ITD (Internal tandem duplications) and/or mutant-NPM1 status as molecular markers of leukemic colonies. The ratio of leukemic colonies (FLT3-ITD and/or mutant-NPM1 positive) versus wild-type (normal, negative for FLT3-ITD and/or mutant-NPM1) was determined for control (untreated) and samples treated with IMGN779 at doses of 100 pM and 1000 pM. Treatment with IMGN779 at the 1000 pM concentration eliminated LSCs, while sparing hematopoietic stem cells, as indicated by the presence of normal colonies only (FIG. 4A).

FIG. 4B shows that after 5 weeks, there was a dose-dependent increase in colony number. Colonies were analyzed at 7 weeks for the presence of molecular markers of AML (Trisomy 8, FLT3-ITD and NPM1). The absence of AML molecular markers indicated that wild-type (WT) colonies were derived from normal HSCs. Increased colony formation was also observed in long-term cultures of normal bone marrow after treatment with IMGN779, indicating that hematopoietic stem cells (HSCs) are spared. Thus, IMGN779 caused a dose-dependent decrease of leukemic colony formation and an increase in normal hsc colonies in long-term leukemic stem cell cultures.

### Example 5

### P-Glycoprotein (PGP) Expressing Cells are Sensitive to IMGN779

To assess the role of PGP, the in vitro activity of IMGN779 was tested with and without the addition of 2 μM of the PGP inhibitor PSC833 Inhibition of PGP resulted in potentiation of in vitro activity for IMGN779 ranging from 0.8 to 29-fold and was highest in the two AML samples that were least sensitive to IMGN779 (5 and 29 fold). In the remaining samples potentiation was less than factor 5. PGP activity inversely correlated with CD33-expression levels (FIG. 5A) and IMGN779 cytotoxicity (FIG. 5B).

### Example 6

### IMGN779 Demonstrates Highly Potent and CD33 Specific In Vitro Cytotoxicity Against Primary Patient AML Cells

Assays for IMGN779 cytotoxicity against primary patient AML cells were carried out in a short-term liquid culture assay. The highest IMGN779 activity was generally observed with CD33 expression levels >5000 antigens per cell (FIG. 2). IMGN779 activity was CD33-specific (FIG. 5A). Non-targeted DGN462-ADC was not active (no IC₅₀ reached at highest dose tested in 33/35 samples). CD33 levels ranged from ˜200 to 16,000 antigens per cell.

### Example 7

### AML Cell Lines are Highly Sensitive to IMGN779 and DGN462

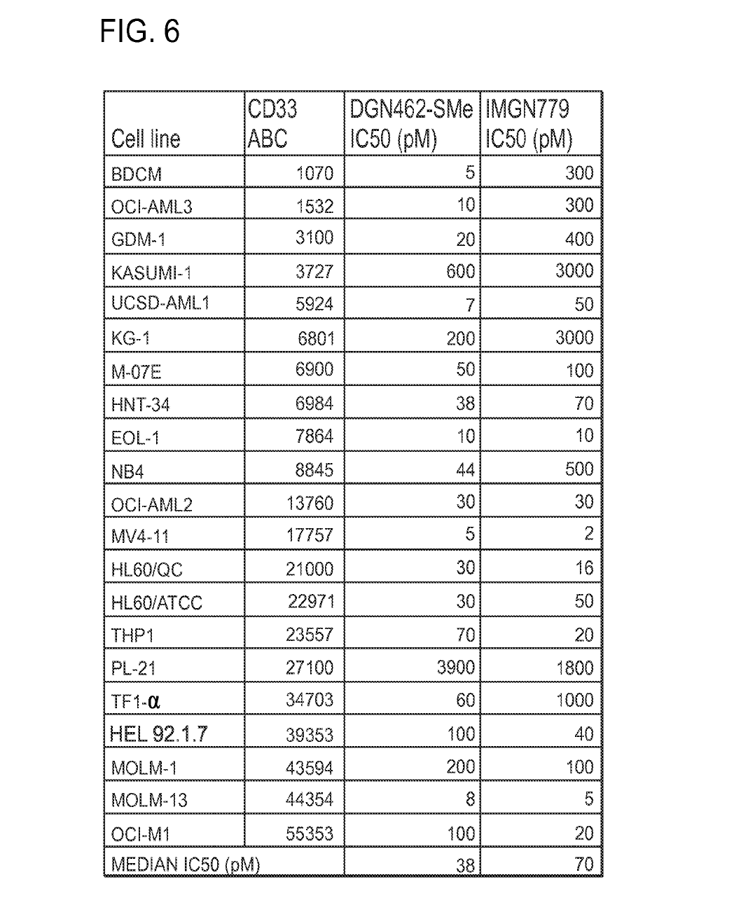

A panel of 21 AML cell lines were evaluated in vitro (FIG. 6). CD33 expression ranged from 1,000-55,000 antigens per cell. These levels were much higher than levels detected in primary patient cells. The median sensitivity to the free drug DGN462-SMe was 38 pM (ranging from 5 to 3900 pM IC₅₀). Median sensitivity to IMGN779 was 70 pM (ranging from 2 to 3000 pM IC₅₀).

CD33 levels on AML cell lines were measured using a calibrated quantitative flow cytomety method. Cells were stained with a phycoerythrin (PE)-conjugated anti-CD33 antibody (BD Biosciences) and compared with a BD Quantibrite bead calibration curve. Cells were plated in 96-well tissue culture plates at a density of 2,000 to 5,000 cells per well, and incubated with various concentrations of DGN462-SMe or IMGN779 for 5 days at 37° C. Survival of the cells was determined using the WST-8 based colorimetric assay (Dojindo Molecular Technologies, Inc.).

### Example 8

### IMGN779 is Highly Active and Antigen Specific Against Human AML Xenografts at a Minimally Efficacious Dose of 0.6 mg/kg

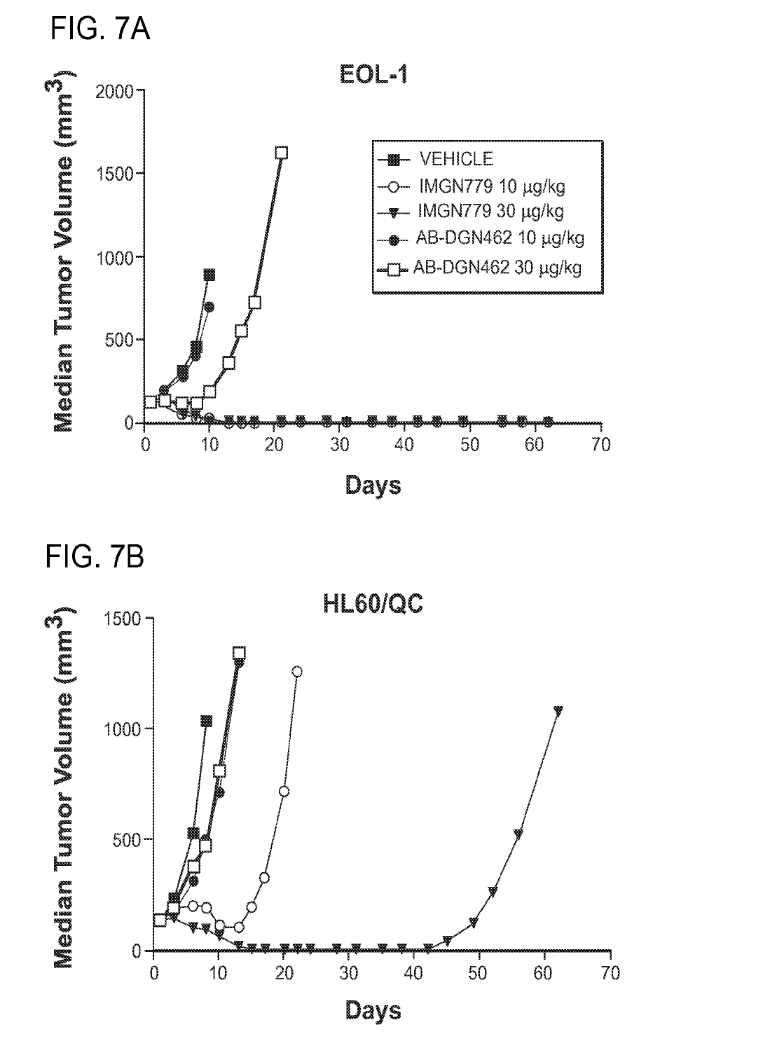

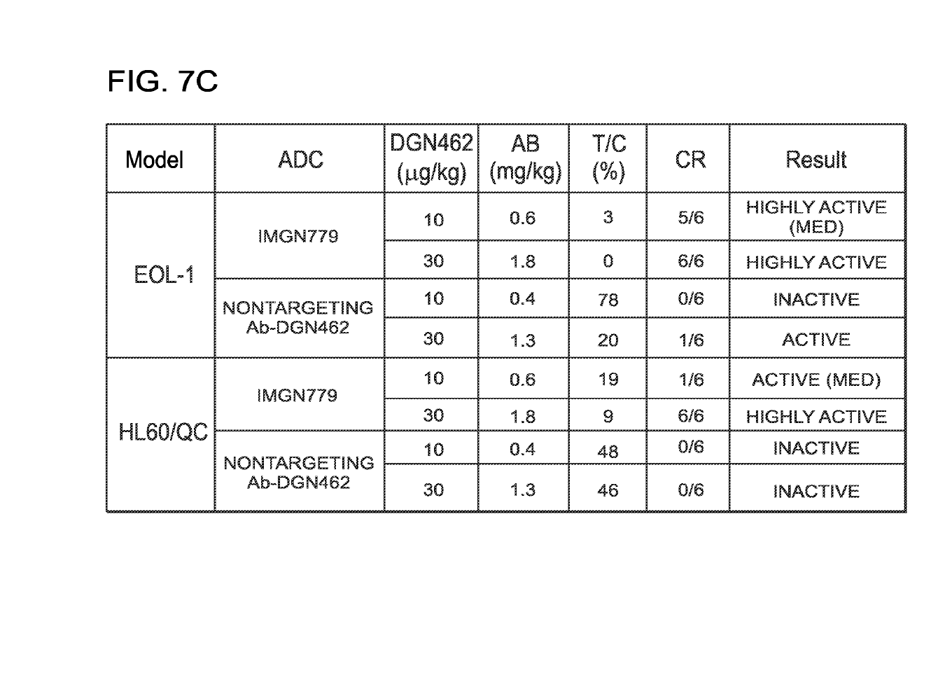

To determine the antitumor activity of IMGN779, SCID mice bearing EOL-1 acute myeloid leukemia (AML)(FIG. 7A) or HL60/QC promyelocytic leukemia (PML)(FIG. 7B) cells in subcutaneous xenografts (˜100 mm³) received a single intravenous injection of IMGN779. Tumor growth inhibition (T/C %) was calculated as the ratio of median tumor volumes of treated (T) and control (C) groups at the day when control median tumor volume was ˜1000 mm³ (Bissery, M. et al., Cancer Res. 51, 4845-4852, September 1991). According to the National Cancer Institute standards, a T/C≦42% is the minimum level of anti-tumor activity. A T/C<10% is considered a high anti-tumor activity level. FIG. 7C is a table summarizing the data obtained from the xenograft models.

### Example 9

### IMGN779 is Well Tolerated in CD-1 Mice, No Hepatotoxicity or Delayed Toxicity

To determine tolerability and toxicity of IMGN779, female CD-1 mice (7 weeks of age) were injected intravenously with IMGN779 at the doses described. Body weight was measured daily. Toxicity was assessed at maximum tolerated dosage (MTD) and ˜30% of the MTD by measurement of serum chemistries, including liver enzymes alanine aminotransferase (ALT) and aspartate aminotransferase (AST), at day 5 (body weight loss nadir) post dosing.

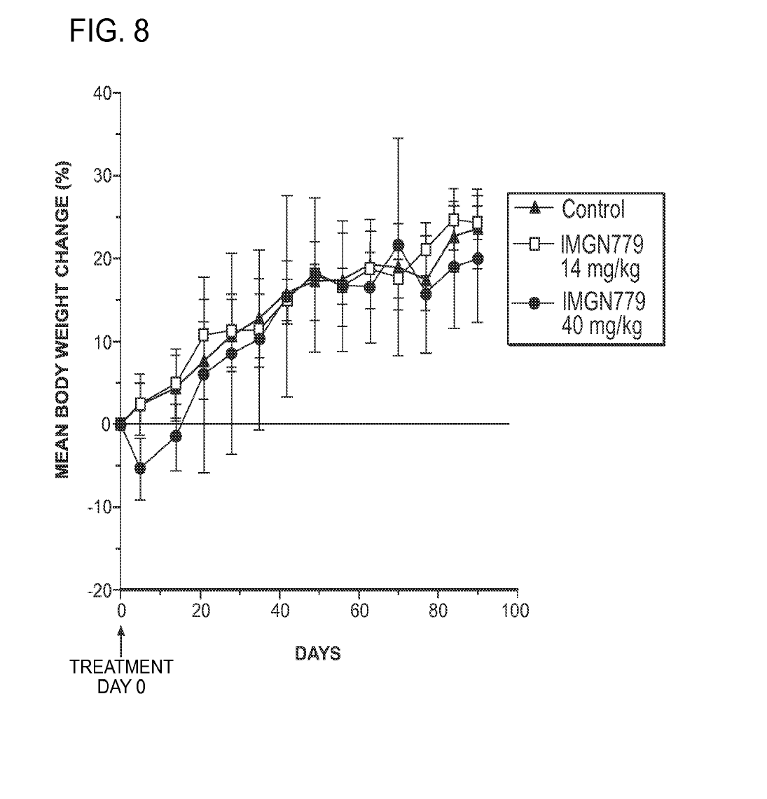

IMGN779 did not cause liver toxicity in mice at the maximum tolerated (MTD) dose. Alanine aminotransferase (ALT)/aspartate aminotransferase (AST) values were comparable to normal reference ranges for CD-1 mice. No evidence of delayed toxicity was observed with antibody drug conjugates (ADCs) containing DNA-crosslinking agents. See FIG. 8.

### Example 10

### IMGN779 has Pharmacokinetic Profile and In Vivo Stability Comparable to Antibody Drug Conjugates (ADCs) with Cleavable Linkers, Conjugate Bioactivity is Maintained for at Least 3 Days

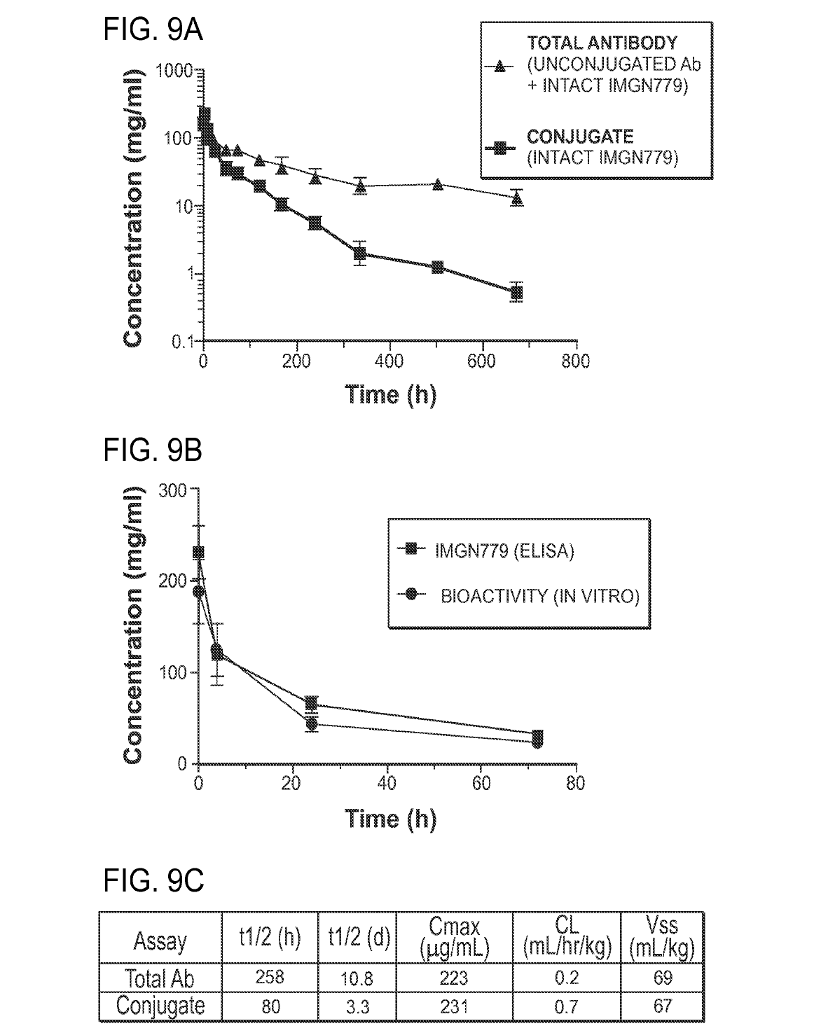

To determine pharmacokinetics and bioactivity of IMGN779, plasma concentrations of total antibody (Ab) (both unconjugated Ab and intact antibody drug conjugate) (FIG. 9A) and intact conjugate were determined by ELISA (FIG. 9B) after a single intravenous injection of IMGN779 (5 mg/kg) in CD-1 mice. Pharmacokinetic (PK) analysis was performed using the non-compartmental analysis program (201), WinNonlin, Professional version 6.1 (Pharsight, Mountain View, Calif.). The biologically active concentration of IMGN779 in mouse plasma was determined using a cytotoxicity assay. See FIGS. 9B and 9C. Cells were exposed to either serial dilutions of a standard IMGN779 sample, or to titrated plasma samples from mice dosed with conjugate. The biologically active concentration of IMGN779 in mouse plasma was determined by multiplying the IC50 dilution for each plasma sample by the IC₅₀ of the IMGN779 standard.

### Example 11

### IMGN779 Exhibited High In Vitro Cytotoxicity Against Primary Patient AML Cells with FLT3-ITD Mutations

Potency of IMGN779 against primary AML samples from patient blood and bone marrow was evaluated. Colony formation assays after 24-hour exposure were used to assess the cytotoxic activity of IMGN779 on leukemic progenitors. CD33 expression on primary patient AML cells was measured using a calibrated flow cytometry method. AML samples were stained with a fluorescent-tagged anti-CD33 antibody and compared with the fluorescent signal of a calibration curve. The calibration curve was generated using fluorescent-tagged beads at varying label to bead ratio, allowing the total number of CD33 antibodies bound per AML cell (ABC value) to be determined AML blast cells were gated on SSC and CD45 antibody staining.

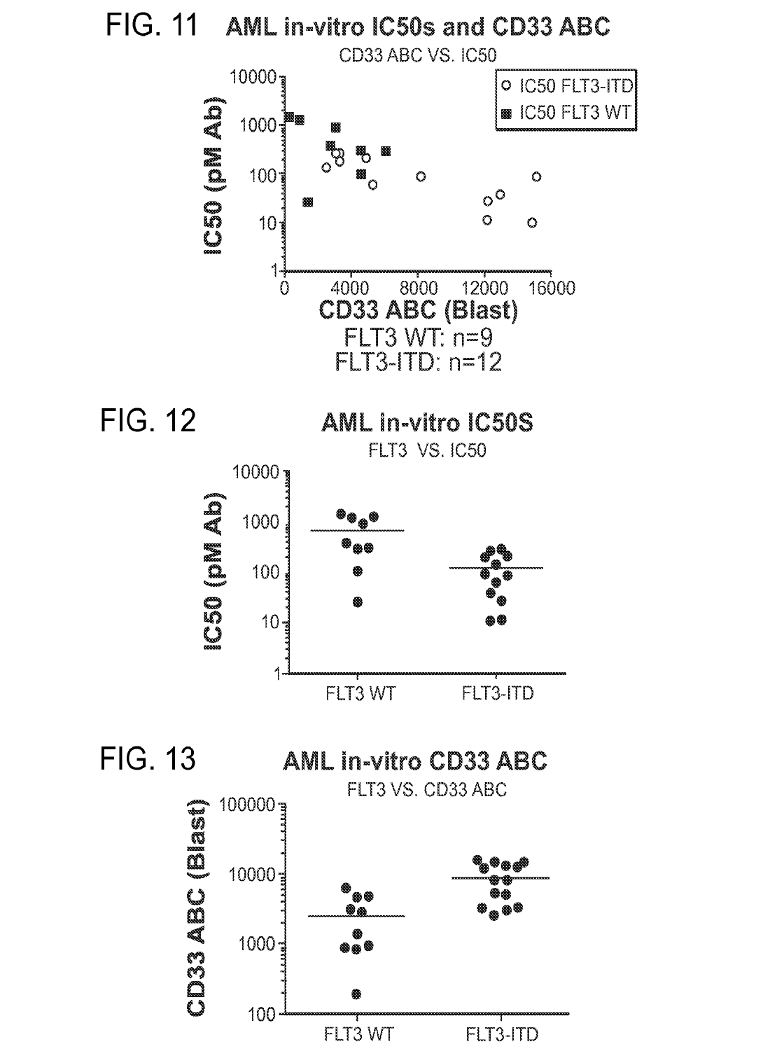

CD33 was expressed at levels ranging from about 200-15,000 ABC in patient AML blast cells. IMGN779 had high cytotoxic activity against patient AML cells with IC₅₀ values ranging from 11 pM to 1.6 nM, with a relationship to CD33 expression level (FIG. 11).

Genomic testing results were available for 21 of the primary AML samples that had IC₅₀ values generated. Of these, 12 carried a FLT3 internal tandem duplication mutation (FLT3-ITD). The mean IC₅₀ values for the FLT3-ITD samples was lower than for the other samples tested (FIG. 12). This indicated that IMGN779 was highly active in vitro in primary patient FLT3-ITD AML samples. The mean CD33 ABC was higher for the FLT3-ITD samples than for the other samples tested (FIG. 13). Without intending to be bound by theory, this may contribute in part to their relative sensitivity.

### Example 12

### IMGN779 Exhibited High In Vitro Cytotoxicity Against AML Cell Lines with FLT3-ITD Mutations

Cytotoxic potency of IMGN779 in AML cell lines was evaluated using continuous exposure up to 7 days, with WST-8 viability staining. CD33 ABC levels were measured using a calibrated flow cytometry method. FLT3 status of the cell lines tested was reported in the catalogue of somatic mutations in cancer (COSMIC) database, and confirmed by a sequencing study.

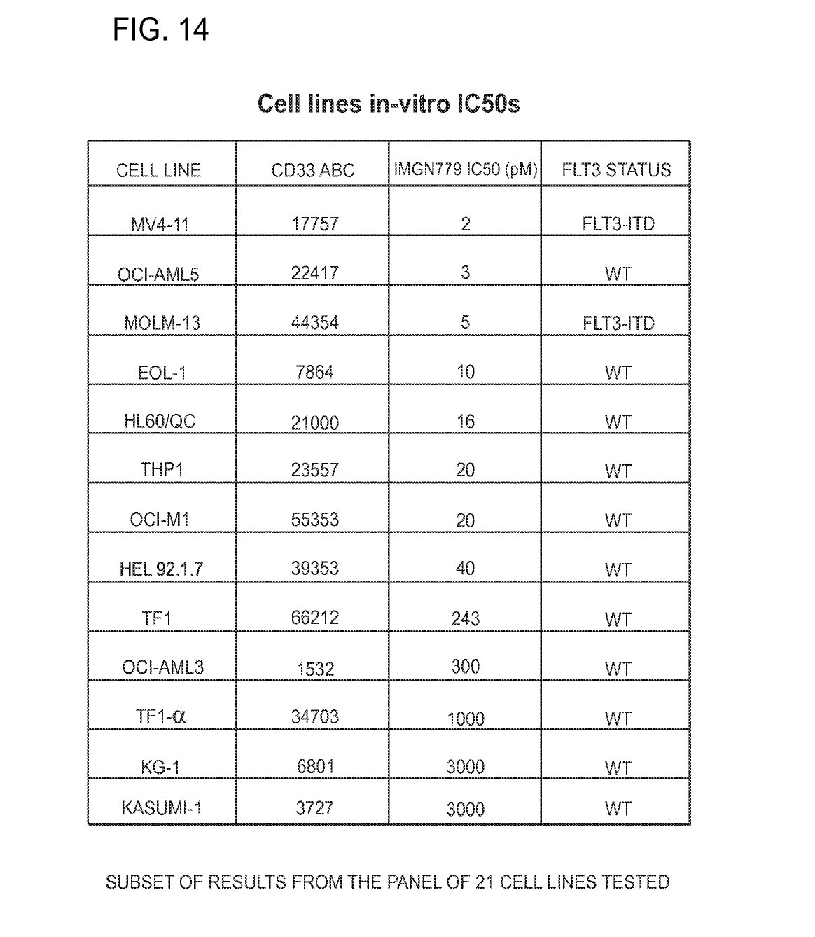

IMGN779 had high cytotoxic activity against AML cell lines with IC₅₀ values ranging from 2 pM to 3 nM. The IC₅₀ values for the two cell lines MV4-11 and MOLM-13 with FLT3-ITD mutations were 2 and 5 pM, respectively, indicating that IMGN779 was highly active in vitro against FLT3-ITD AML cell lines (FIG. 14).

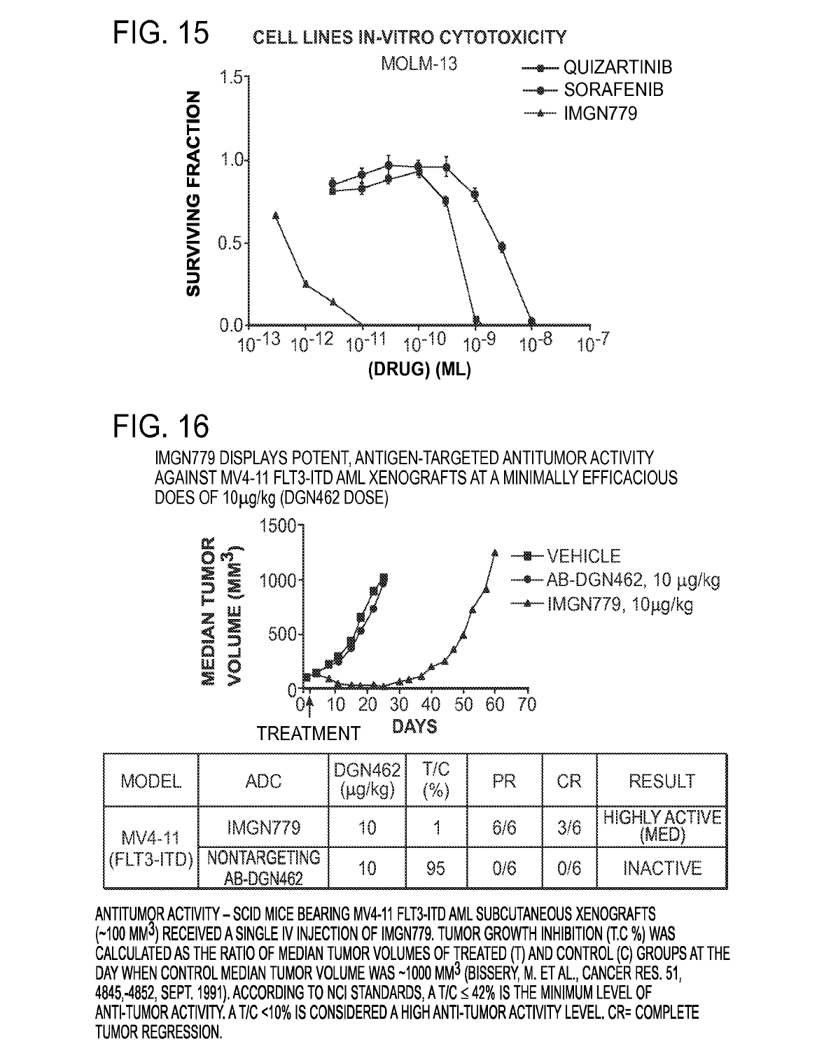

In addition to IMGN779, Sorafenib and Quizartinib were also assessed for cytotoxic potency in FLT3-ITD AML cell lines using continuous exposure up to 7 days, with WST-8 viability staining. Sorafenib is a small molecular inhibitor of several tyrosine protein kinases and Quizartinib is a small molecule kinase inhibitor that specifically targets class III receptor tyrosine kinases, including FLT3. Both are used to treat FLT3-ITD AML patients in clinical trials. The IC₅₀ value for IMGN779 was lower than the IC₅₀ values of Sorafenib and Quizartinib in the MOLM13 cell line (FIG. 15), indicating that IMGN779 is highly active in FLT3-ITD AML cell lines compared to other relevant compounds.

### Example 13

### IMGN779 Displayed Potent, Antigen-Targeted Antitumor Activity Against MV4-11 FLT3-ITD AML Xenografts at a Minimally Efficacious Dose

The anti-tumor activity of IMGN779 was evaluated in an established subcutaneous xenograft model of FLT3-ITD AML. SCID mice (n=24) were inoculated with MV4-11 human FLT3-ITD AML cells (1×10⁷ cells/animal) injected subcutaneously into the right flank of the mice. When the tumors reached about 100 mm³ in size (˜13 days after tumor cell inoculation), FcR blocking with excess human IgG was initiated using chKTi antibody administered at a dose of 400 mg/kg on day 0 (day 13 post inoculation) and at a dose of 100 mg/kg on days 5 and 10 (days 18 and 23 post inoculation). Based on a plasma circulation half-life of about 12 days in mice, plasma IgG concentrations should be maintained at approximately 10 mg/mL. This plasma concentration is comparable to human circulating IgG levels, and should be sufficient to block all FcR present on the MV4-11 cells. On day 14 post inoculation, the mice were randomly divided into treatment groups of 6 animals each based on tumor volume (approximately 100 mm³), and treated with a single intravenous injection of IMGN779 or a nontargeted control chKTi-sulfo-SPDB-DGN462 at a dose of 10 μg/kg, based on DGN462 concentration. Tumor growth inhibition (T/C %) was calculated as the ratio of median tumor volumes of treated (T) and control (C) groups at the day when control median tumor volume was ˜1000 mm³ (Bissery, M. et al., Cancer Res. 51, 4845-4852, September 1991). According to NCI standards, a T/C≦42% is the minimum level of anti-tumor activity. A T/C<10% is considered a high anti-tumor activity level.

Treatment with IMGN779 at 10 μg/kg had high antitumor activity against MV4-11 xenografts, (T/C=1%) with partial regressions (PR) in 6/6 animals and complete regression (CR) in 3/6 animals (FIG. 16). Treatment with a matched dose of the nontargeted control conjugate, chKTi-sulfo-SPDB-DGN462, was inactive (T/C=95%) with no tumor regressions.

The results of this study demonstrate that IMGN779, targeting CD33, was highly active at a dose of 10 μg/kg against MV4-11 FLT3-ITD AML xenografts.

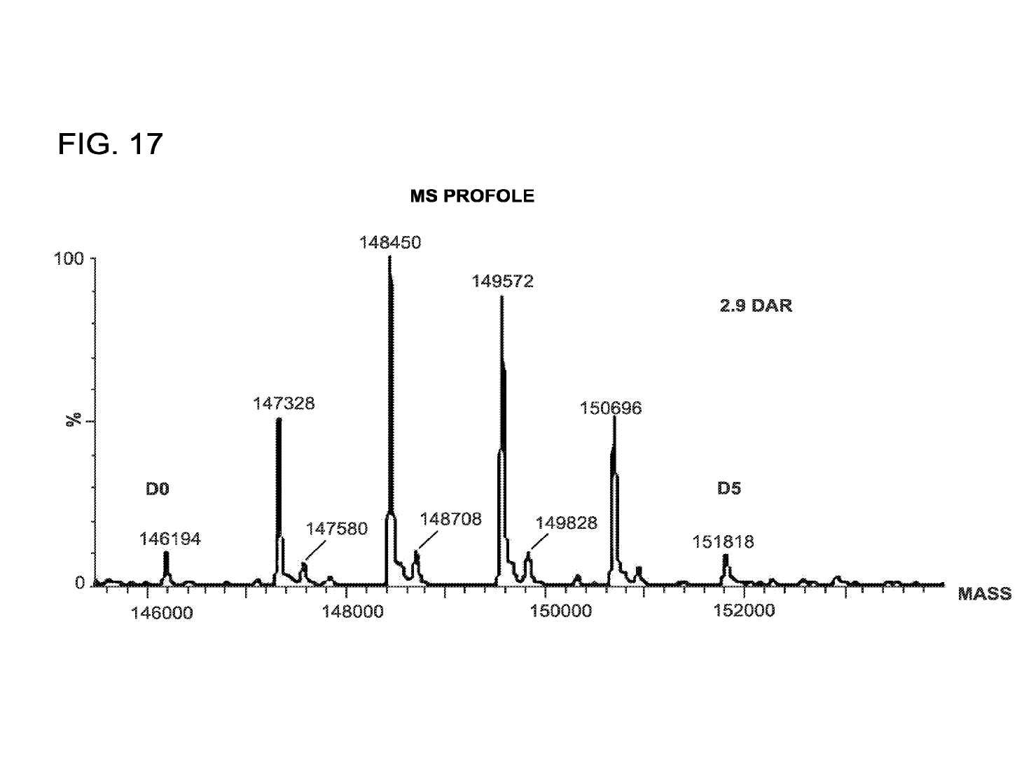

In sum, IMGN779 is a CD33-targeted antibody drug conjugate (ADC) utilizing a novel DNA-alkylating agent, DGN462. The mass spectrometer profile indicates that there are approximately three DGN462 molecules per antibody (FIG. 17). Its favorable preclinical tolerability profile indicates that IMGN779 likely confers a therapeutic advantage over existing clinical agents for AML that demonstrate activity, but that have significant toxicity. The highly potent, CD33-targeted activity of IMGN779 against AML cell lines and primary patient AML cells in vitro, the anti-tumor activity observed against AML xenografts in mice and the favorable safety profile indicate that it is a promising treatment for AML.

The results described herein above were obtained using the following methods and materials.

### In Vitro Methods

CD33 Quantitation & Pgp Activity

Primary patient AML cells from bone marrow or peripheral blood were analyzed by flow cytometry. AML samples (CD34⁺/CD38⁺/CD33⁺ progenitor compartment) were stained with a phycoerythrin (PE)-conjugated anti-CD33 antibody (BD Biosciences) and compared with a BD Quantibrite bead calibration curve. The functional activity of Pgp was calculated by the ratio of the mean fluorescence intensity (MFI) of Syto16±PSC833 Pgp inhibitor.

In Vitro Potency on Primary AML Cells

Cells (or normal human bone marrow samples) were exposed to various concentrations of IMGN779 or non-targeting ADC control for 24 hours. Samples were divided into a short-term liquid culture (STLC) assay to measure the cytotoxicity toward AML progenitor cells, and long-term liquid culture (LTLC) assay to measure the effect on LSCs and normal HSCs. STLC was used to measure colony forming units 10-14 days in cells following plating in semi-solid MethoCult H4230 medium (Stemcell technologies). The LTLC assays were performed similarly with the addition of growth factors for long-term culture 5-7 weeks. In both assays, colonies were counted to determine colony forming units per number of cells initially plated. LTLC colonies were further analyzed for the presence of AML molecular markers using PCR or FISH.

In Vitro Potency on Cell Lines

Cells were plated in 96-well tissue culture plates at a density of 2,000 to 5,000 cells per well, and incubated with various concentrations of DGN462-SMe or IMGN779 for 5 days at 37° C. Survival of the cells was determined using the WST-8 based colorimetric assay (Dojindo Molecular Technologies, Inc.).

Antitumor Activity

SCID mice bearing HL60/QC and EOL-1 acute myeloid leukemia (AML) subcutaneous xenografts (˜100 mm³) received a single IV injection of IMGN779. Tumor growth inhibition (T/C %) was calculated as the ratio of median tumor volumes of treated (T) and control (C) groups at the day when control median tumor volume was ˜1000 mm³ (Bissery, M. et al., Cancer Res. 51, 4845-4852, September 1991). According to NCI standards, a T/C≦42% is the minimum level of anti-tumor activity. A T/C<10% is considered a high anti-tumor activity level. CR=complete tumor regression.

Tolerability/Toxicity

Female CD-1 mice (7 weeks) were injected intravenously (IV) with IMGN779 at the doses described. Body weight was measured daily. Toxicity was assessed at MTD and ˜30% MTD doses by measurement of serum chemistries, including liver enzymes alanine aminotransferase (ALT) and aspartate aminotransferase (AST) at day 5 (body weight loss nadir) post dosing.

Pharmacokinetics and Bioactivity

Plasma concentrations of total Ab (both unconjugated Ab and intact ADC) and intact conjugate were determined by ELISA after a single IV injection of IMGN779 (5 mg/kg) in CD-1 mice. Pharmacokinetic (PK) analysis was performed using the non-compartmental analysis program (201), WinNonlin, Professional version 6.1 (Pharsight, Mountain View, Calif.). The biologically active concentration of IMGN779 in mouse plasma was determined using a cytotoxicity assay. Cells were exposed to either serial dilutions of a standard IMGN779 sample, or to titrated plasma samples from mice dosed with conjugate. The biologically active concentration of IMGN779 in mouse plasma was determined by multiplying the IC₅₀ dilution for each plasma sample by the IC₅₀ of the IMGN779 standard.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The present invention may be related to subject matter described in U.S. Pat. Nos. 7,557,189; 7,342,110; 8,119,787; 8,337,855; and U.S. patent application Ser. No. 13/680,614, which disclose the full sequence of the anti-CD33 antibody (huMy9-6), each of which is incorporated herein by reference. All patents and publications mentioned in this specification are herein incorporated by reference to the same extent as if each independent patent and publication was specifically and individually indicated to be incorporated by reference.

What is claimed is:

1. A method of treating acute myeloid leukemia in a subject, the method comprising administering an effective amount of an immunoconjugate to a pre-selected subject, wherein the immunoconjugate comprises a humanized or chimeric antibody or fragment thereof conjugated to a cytotoxic benzodiazepine dimer compound via a cleavable disulfide linker represented by the following structural formula: 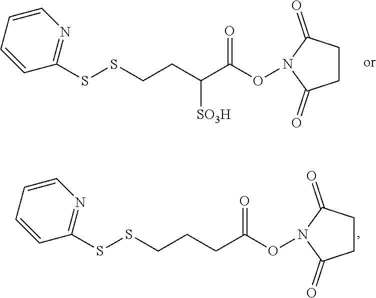 [SMILES: O=C(ON1C(=O)CCC1=O)C(CCSSc1ccccn1)S(=O)(=O)O ; O=C(CCCSSc1ccccn1)ON1C(=O)CCC1=O]
wherein the antibody comprises a heavy chain variable region comprising one or more complementarity determining regions selected from the group consisting of SEQ ID NOs: 1-3; and/or a light chain variable region comprising one or more complementarity determining regions selected from the group consisting of SEQ ID NOs: 4-6; and the cytotoxic benzodiazepine dimer compound represented by one of the following structural formulas or a pharmaceutically acceptable salt thereof: 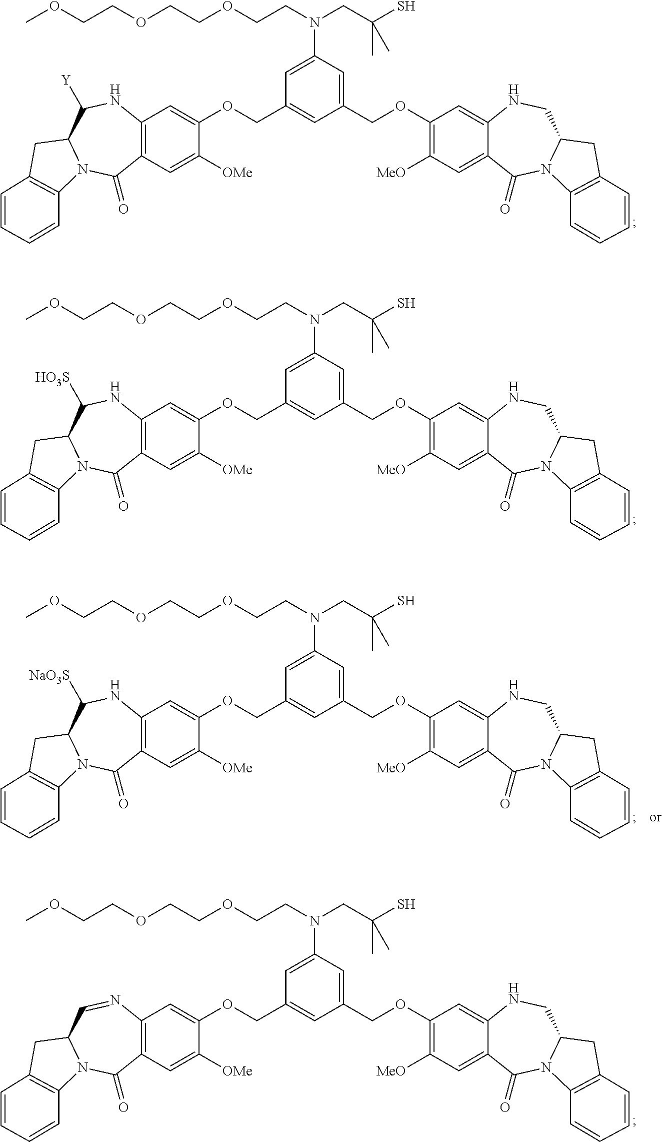
wherein Y is —SO₃M and M is H or a pharmaceutically acceptable cation and wherein the pre-selection comprises detecting CD33 in a biological sample of the subject.

2. A method of treating acute myeloid leukemia in a subject, the method comprising administering an effective amount of an immunoconjugate to a subject determined to have about 1,000 CD33 antigens per cell in a biological sample, wherein the immunoconjugate comprises a humanized or chimeric antibody or fragment thereof conjugated to a cytotoxic benzodiazepine dimer compound via a cleavable disulfide linker represented by the following structural formula: 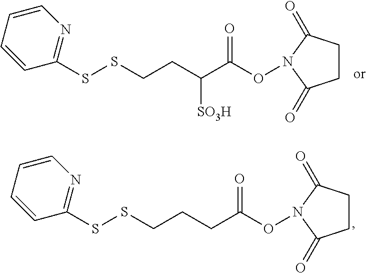
wherein the antibody comprises a heavy chain variable region comprising one or more complementarity determining regions selected from the group consisting of SEQ ID NOs: 1-3; and/or a light chain variable region comprising one or more complementarity determining regions selected from the group consisting of SEQ ID NOs: 4-6; and the cytotoxic benzodiazepine dimer compound represented by one of the following structural formulas or a pharmaceutically acceptable salt thereof: 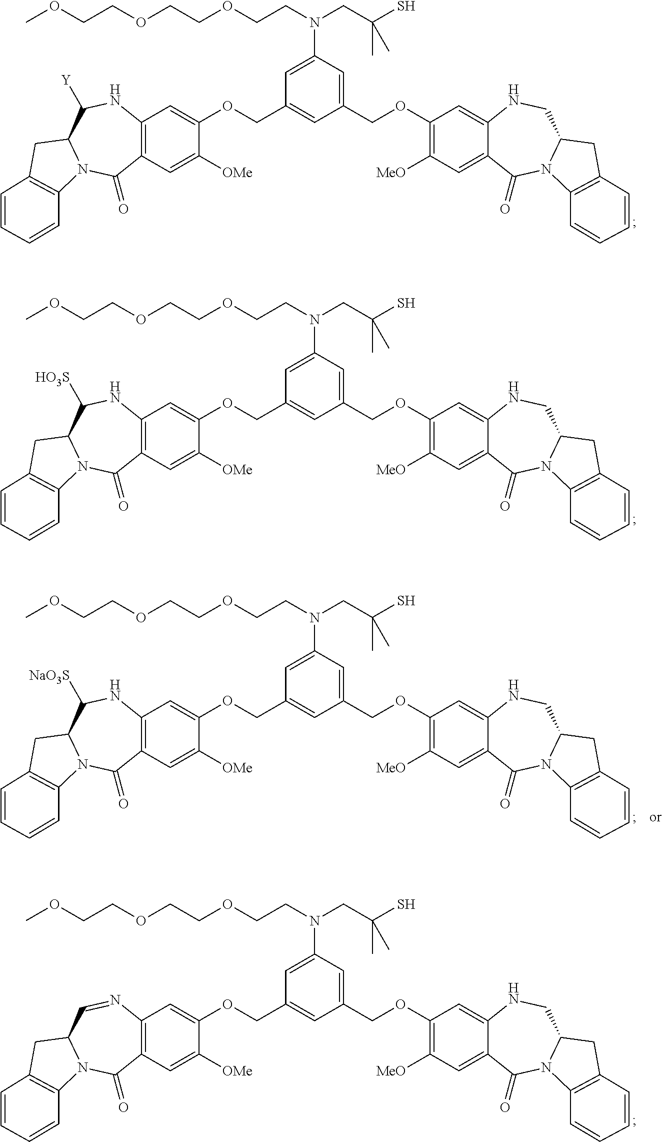
wherein Y is —SO₃M and M is H or a pharmaceutically acceptable cation and wherein the pre-selection comprises detecting CD33 in a biological sample of the subject.

3. The method of claim 1 or 2, wherein the heavy chain variable region comprises an amino acid sequence having at least 95% identity to the amino acid sequence of SEQ ID NO:7 or 9.

4. The method of claim 1 or 2, wherein the light chain variable region comprises an amino acid sequence having at least 95% identity to the amino acid sequence of SEQ ID NO: 8 or 10.

5. The method of claim 1 or 2, wherein the antibody is a humanized or chimeric My9-6 antibody.

6. The method of claim 5, wherein the humanized antibody is a CDR-grafted or resurfaced antibody.

7. The method of claim 1 or 2, wherein the immunoconjugate comprises a humanized My9-6 antibody conjugated to a cytotoxic benzodiazepine dimer compound via N-succinimidyl-4-(2-pyridyldithio)-2-sulfobutanoate, wherein the immunoconjugate is represented by one of the following structural formulas or a pharmaceutically acceptable salt thereof: 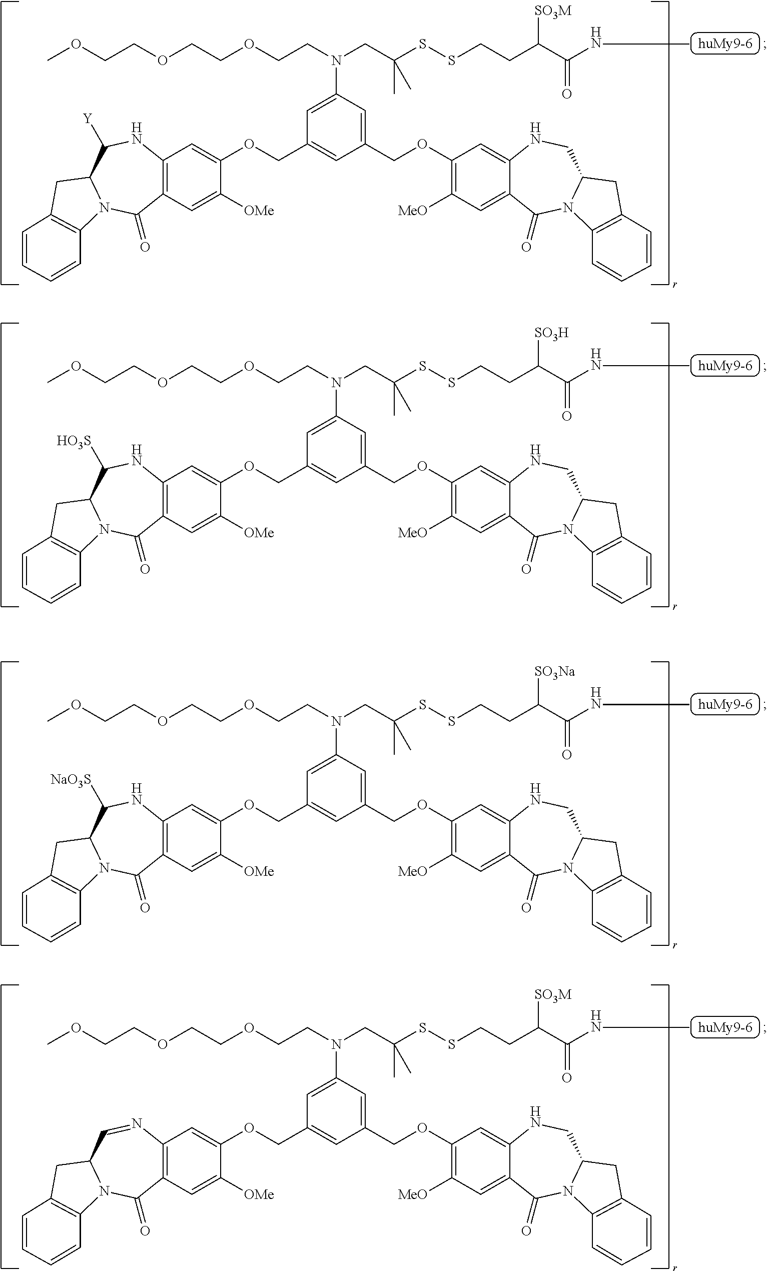 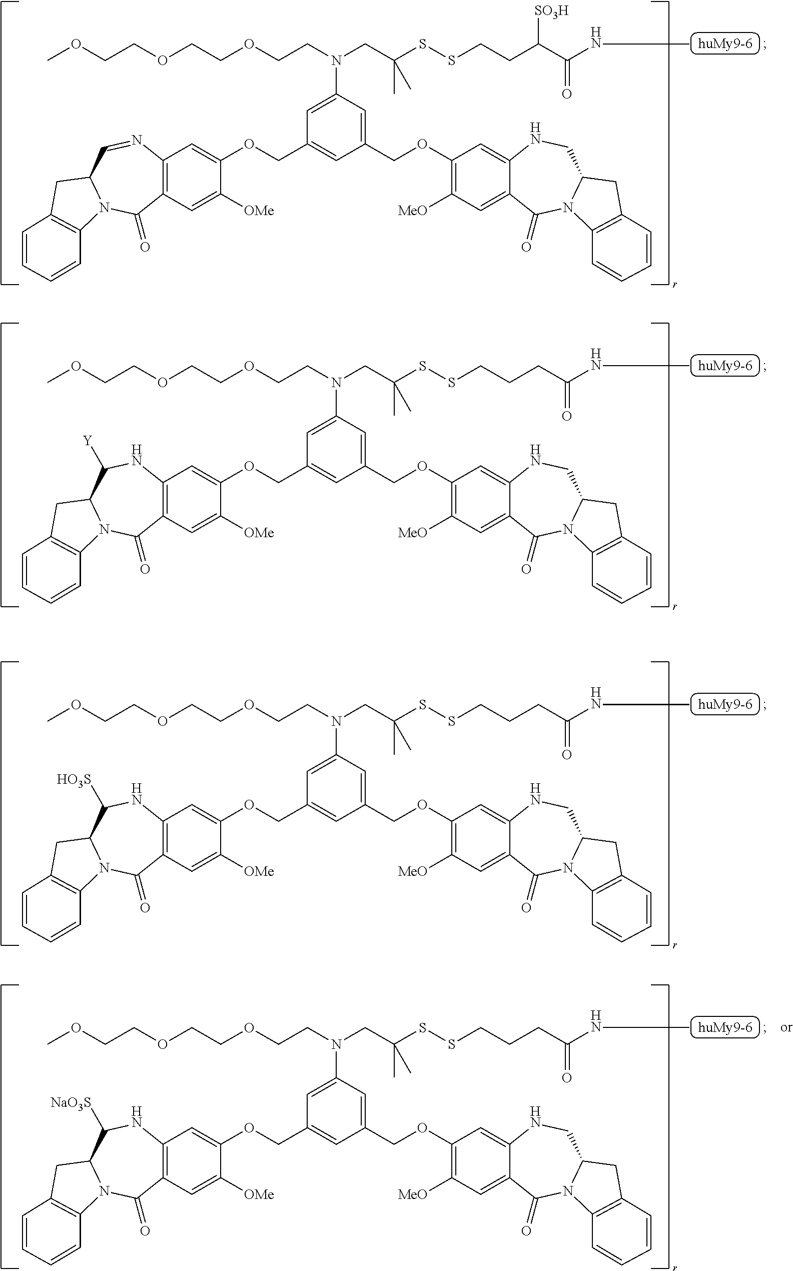 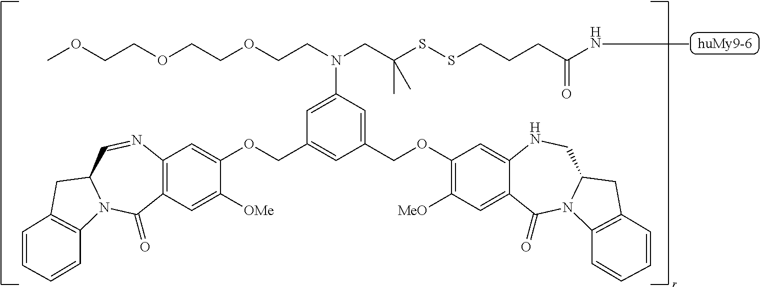
wherein r is an integer from 1 to 10, Y is —SO₃M and M, for each occurrence, is independently —H or a pharmaceutically acceptable cation.

8. The method of claim 1 or 2, wherein the detecting step comprises measuring the level of CD33 present in a peripheral blood or bone marrow sample of the subject, wherein detecting between about 1,000-25,000 antigens per cell pre-selects the subject as likely to respond to the immunoconjugate.

9. The method of claim 8, wherein detecting between about 3,000-25,000 antigens per cell pre-selects the subject as likely to respond to the immunoconjugate.

10. The method of claim 9, wherein detecting between about 5,000-25,000 antigens per cell pre-selects the subject as likely to respond to the immunoconjugate.

11. The method of claim 1 or 2, wherein the detecting step comprises measuring the level of CD33 present in a peripheral blood or bone marrow sample of the subject, wherein detecting at least about 1,000, 3,000, or 5,000 antigens per cell pre-selects the subject as likely to respond to the immunoconjugate.

12. The method of claim 1 or 2, wherein the subject is newly diagnosed with acute myeloid leukemia.

13. The method of claim 1 or 2, wherein the subject is diagnosed with acute myeloid leukemia relapse or with refractory acute myeloid leukemia.

14. The method of claim 13, wherein a sample from the subject diagnosed with acute myeloid leukemia relapse or with refractory acute myeloid leukemia comprises at least about 3,000 antigens per cell.

15. A method of treating a subject having FLT3-ITD positive acute myeloid leukemia, the method comprising administering an effective amount of an immunoconjugate to a pre-selected subject, wherein the immunoconjugate comprises a humanized or chimeric antibody or fragment thereof conjugated to a cytotoxic benzodiazepine dimer compound via a cleavable disulfide linker represented by the following structural formula: 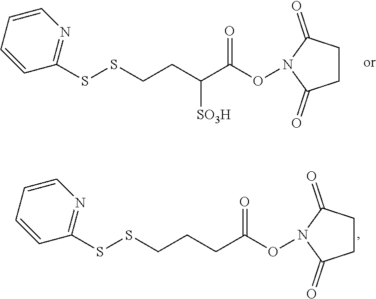 [SMILES: O=C(ON1C(=O)CCC1=O)C(CCSSc1ccccn1)S(=O)(=O)O ; O=C(CCCSSc1ccccn1)ON1C(=O)CCC1=O]
wherein the antibody comprises a heavy chain variable region comprising one or more complementarity determining regions selected from the group consisting of SEQ ID NOs: 1-3; and/or a light chain variable region comprising one or more complementarity determining regions selected from the group consisting of SEQ ID NOs: 4-6; and the cytotoxic benzodiazepine dimer compound represented by one of the following structural formulas or a pharmaceutically acceptable salt thereof: 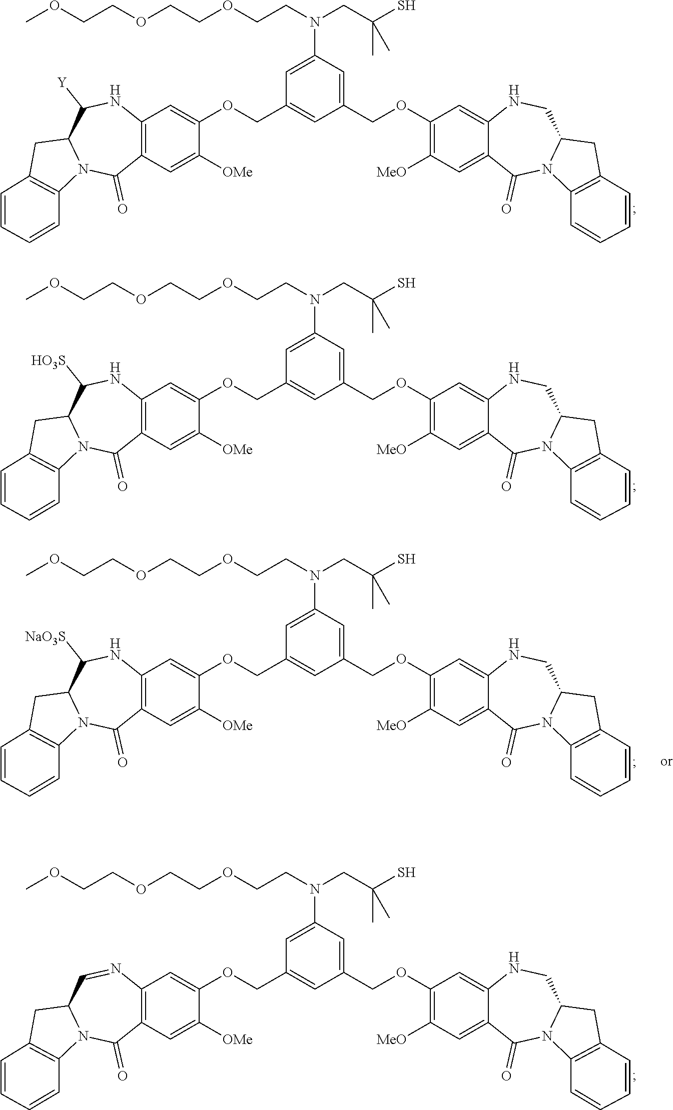
wherein Y is —SO₃M and M is H or a pharmaceutically acceptable cation and wherein the pre-selection comprises detecting FLT3-ITD in a biological sample of the subject.

16. A method of treating a subject having acute myeloid leukemia, the method comprising administering an effective amount of an immunoconjugate to a pre-selected subject determined to have FLT3-ITD positive acute myeloid leukemia, wherein the immunoconjugate comprises a humanized or chimeric antibody or fragment thereof conjugated to a cytotoxic benzodiazepine dimer compound via a cleavable disulfide linker represented by the following structural formula: 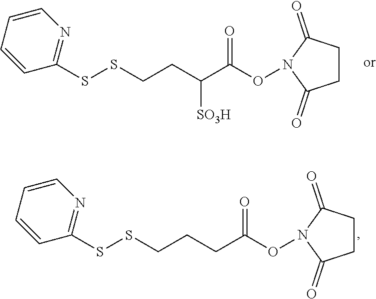 [SMILES: O=C(ON1C(=O)CCC1=O)C(CCSSc1ccccn1)S(=O)(=O)O ; O=C(CCCSSc1ccccn1)ON1C(=O)CCC1=O]
wherein the antibody comprises a heavy chain variable region comprising one or more complementarity determining regions selected from the group consisting of SEQ ID NOs: 1-3; and/or a light chain variable region comprising one or more complementarity determining regions selected from the group consisting of SEQ ID NOs: 4-6; and the cytotoxic benzodiazepine dimer compound represented by one of the following structural formulas or a pharmaceutically acceptable salt thereof: 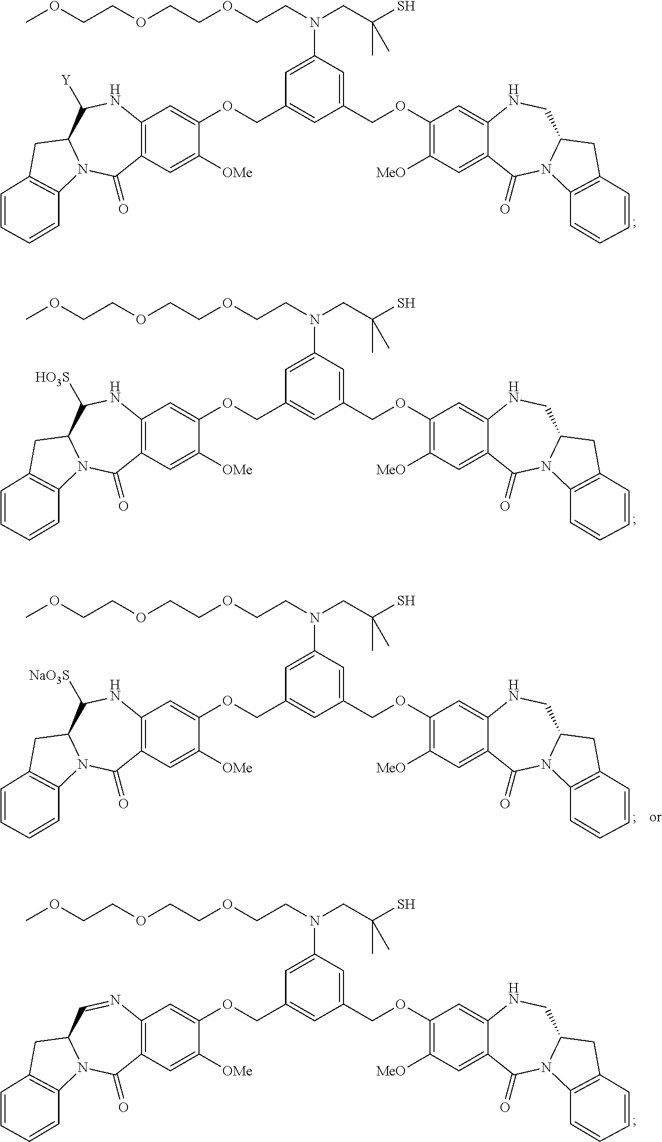
wherein Y is —SO₃M and M is H or a pharmaceutically acceptable cation and wherein the pre-selection comprises determining the FLT3-ITD status in a biological sample of the subject.

17. The method of claim 15 or 16, wherein said biological sample is a peripheral blood or bone marrow sample from said subject.

18. The method of claim 17, wherein said determining comprises a nucleic acid hybridization method or a nucleic acid sequencing method.

19. The method of claim 17, wherein said determining comprises PCR, reverse transcriptase PCR, or real time PCR, or combinations thereof.

20. The method of claim 15 or 16, wherein CD33 levels are determined for said subject having a FLT3-ITD positive acute myeloid leukemia.

21. The method of claim 20, wherein said CD33 levels are determined to be between 1,000-25,000 CD33 antigens per cell.

22. The method of claim 21, wherein said CD33 levels are determined to be between 3,000-25,000 CD33 antigens per cell.

23. The method of claim 22, wherein said CD33 levels are determined to be between 5,000-15,000 CD33 antigens per cell.

24. The method of claim 15 or 16, wherein the heavy chain variable region comprises an amino acid sequence having at least 95% identity to the amino acid sequence of SEQ ID NO:7 or 9.

25. The method of claim 15 or 16, wherein the light chain variable region comprises an amino acid sequence having at least 95% identity to the amino acid sequence of SEQ ID NO: 8 or 10.

26. The method of claim 15 or 16, wherein the antibody is a humanized or chimeric My9-6 antibody.

27. The method of claim 26, wherein the humanized antibody is a CDR-grafted or resurfaced antibody.

28. The method of claim 15 or 16, wherein the immunoconjugate comprises a humanized My9-6 antibody conjugated to a cytotoxic benzodiazepine dimer compound via N-succinimidyl-4-(2-pyridyldithio)-2-sulfobutanoate, wherein the immunoconjugate is represented by one of the following structural formulas or a pharmaceutically acceptable salt thereof: 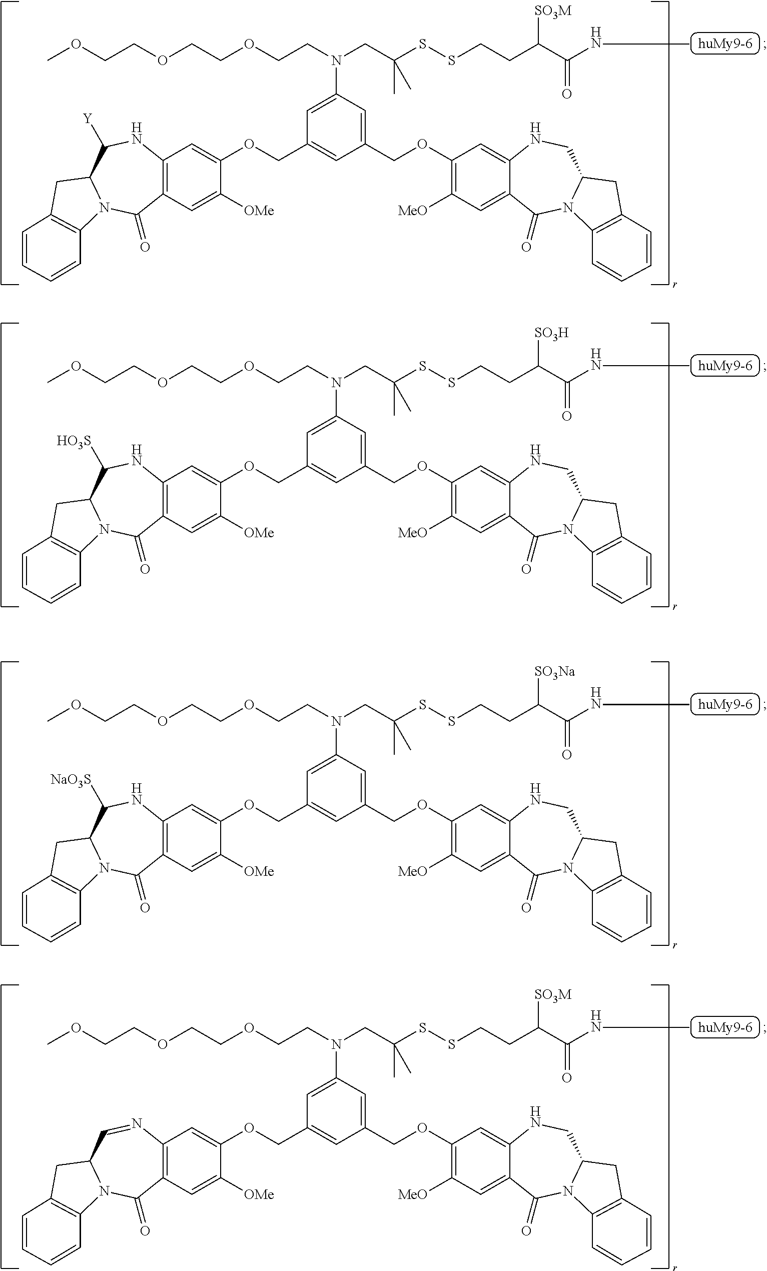 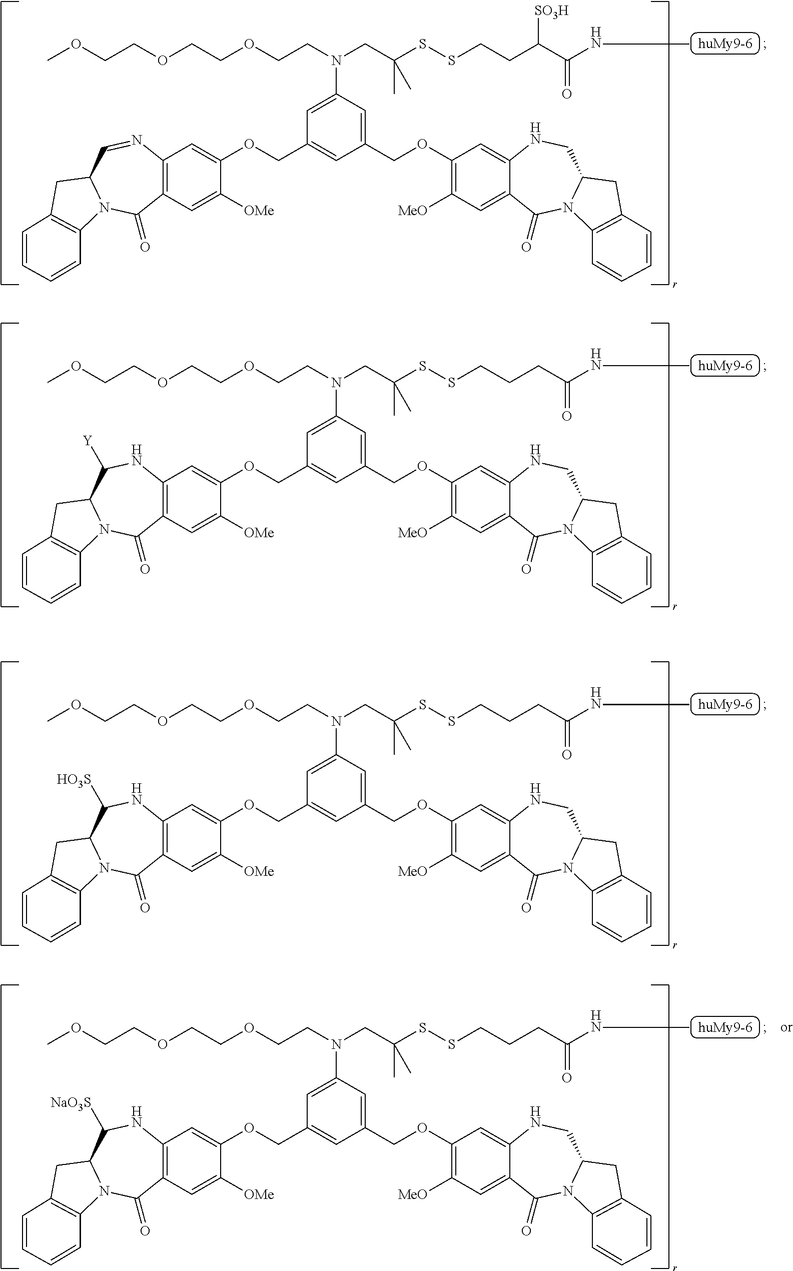 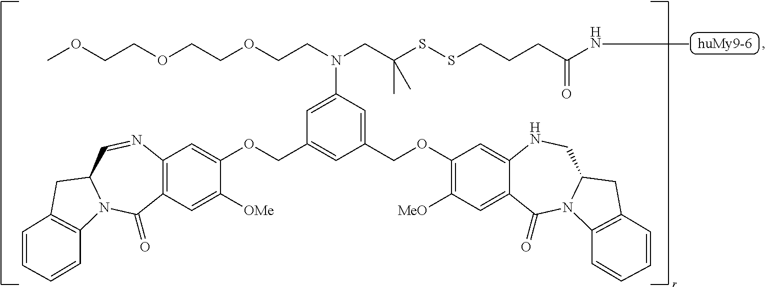
wherein r is an integer from 1 to 10, Y is —SO₃M and M, for each occurrence, is independently —H or a pharmaceutically acceptable cation.

29. A method of identifying a subject as being responsive to treatment with an immunoconjugate, the method comprising:
(a) detecting FLT3-ITD in a biological sample from said subject, and
(b) correlating the detection of FLT3-ITD with responsiveness of the subject to treatment, wherein the presence of FLT3-ITD in the biological sample identifies the subject as responsive to treatment with said immunoconjugate,
wherein the immunoconjugate comprises a humanized or chimeric antibody or fragment thereof conjugated to a cytotoxic benzodiazepine dimer compound via a cleavable disulfide linker represented by the following structural formula: 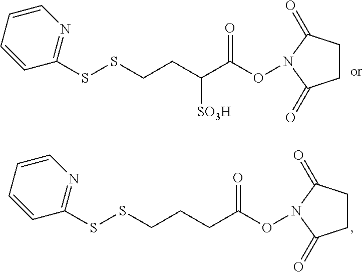
wherein the antibody comprises a heavy chain variable region comprising one or more complementarity determining regions selected from the group consisting of SEQ ID NOs: 1-3; and/or a light chain variable region comprising one or more complementarity determining regions selected from the group consisting of SEQ ID NOs: 4-6; and the cytotoxic benzodiazepine dimer compound represented by one of the following structural formulas or a pharmaceutically acceptable salt thereof: 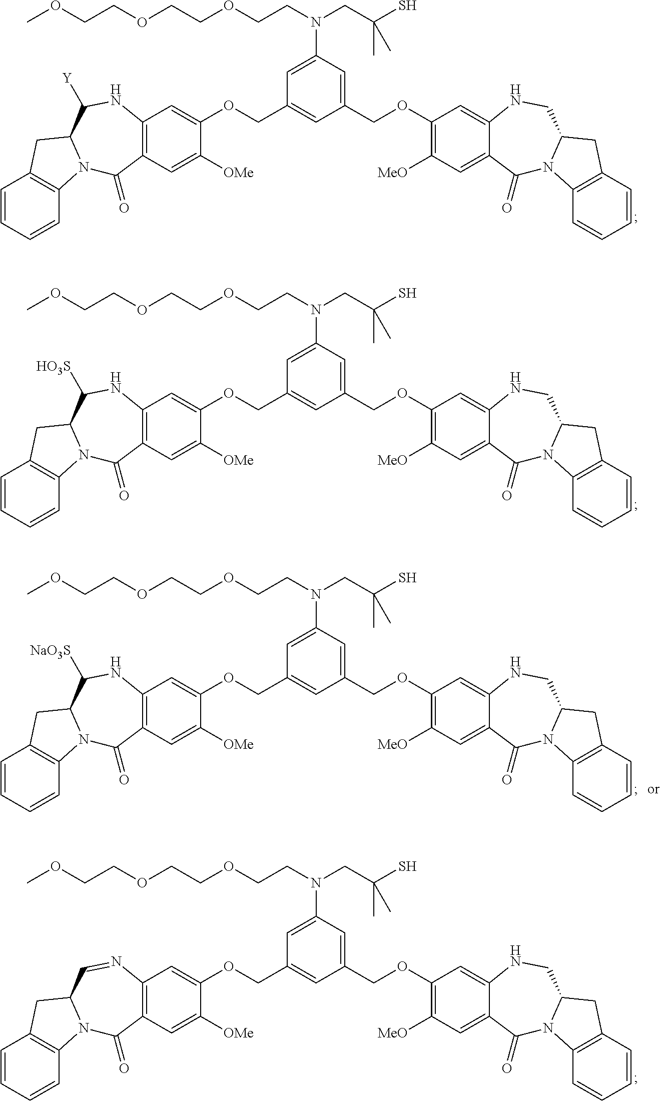
wherein Y is —SO₃M and M is H or a pharmaceutically acceptable cation.

30. The method of claim 29, wherein said method further comprises detecting CD33 levels in a cell of said subject.

31. The method of claim 30, wherein detecting at least about 1,000 CD33 antigens per cell identifies the subject as responsive to treatment with the immunoconjugate.

32. The method of claim 31, wherein detecting at least about 3,000 CD33 antigens per cell identifies the subject as responsive to treatment with the immunoconjugate.

33. The method of claim 32, wherein detecting at least about 5,000 CD33 antigens per cell identifies the subject as responsive to treatment with the immunoconjugate.

34. The method of any one of claims 15-33, wherein the subject is newly diagnosed with acute myeloid leukemia, identified as having acute myeloid leukemia relapse, or identified as having refractory acute myeloid leukemia.

35. The method of any one of claims 15-34, wherein the subject having FLT3-ITD positive acute myeloid leukemia is diagnosed with acute myeloid leukemia relapse and has not received prior treatment with a tyrosine kinase inhibitor.

36. The method of claim 35, wherein the tyrosine kinase inhibitor is a FLT3 tyrosine kinase inhibitor.

37. The method of any one of claims 15-34, wherein the subject having FLT3-ITD positive acute myeloid leukemia is diagnosed with acute myeloid leukemia relapse after receiving prior treatment with a tyrosine kinase inhibitor.

38. The method of claim 37, wherein the tyrosine kinase inhibitor is a FLT3 tyrosine kinase inhibitor.

39. The method of any one of claims 15-34, wherein the subject having FLT3-ITD positive acute myeloid leukemia is diagnosed with refractory acute myeloid leukemia and has not received prior treatment with a tyrosine kinase inhibitor.

40. The method of claim 39, wherein the tyrosine kinase inhibitor is a FLT3 tyrosine kinase inhibitor.

41. The method of any one of claims 15-34, wherein the subject having FLT3-ITD positive acute myeloid leukemia is diagnosed with refractory acute myeloid leukemia after receiving prior treatment with a tyrosine kinase inhibitor.

42. The method of claim 41, wherein the tyrosine kinase inhibitor is a FLT3 tyrosine kinase inhibitor.

43. A method for treating or preventing acute myeloid leukemia relapse in a subject, the method comprising administering an effective amount of an immunoconjugate to a pre-selected subject determined to have FLT3-ITD positive acute myeloid leukemia and that has not received prior treatment with a tyrosine kinase inhibitor, wherein the immunoconjugate comprises a humanized or chimeric antibody or fragment thereof conjugated to a cytotoxic benzodiazepine dimer compound via a cleavable disulfide linker represented by the following structural formula: 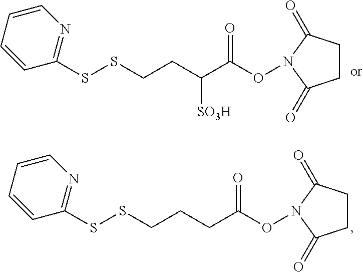
wherein the antibody comprises a heavy chain variable region comprising one or more complementarity determining regions selected from the group consisting of SEQ ID NOs: 1-3; and/or a light chain variable region comprising one or more complementarity determining regions selected from the group consisting of SEQ ID NOs: 4-6; and the cytotoxic benzodiazepine dimer compound represented by one of the following structural formulas or a pharmaceutically acceptable salt thereof: 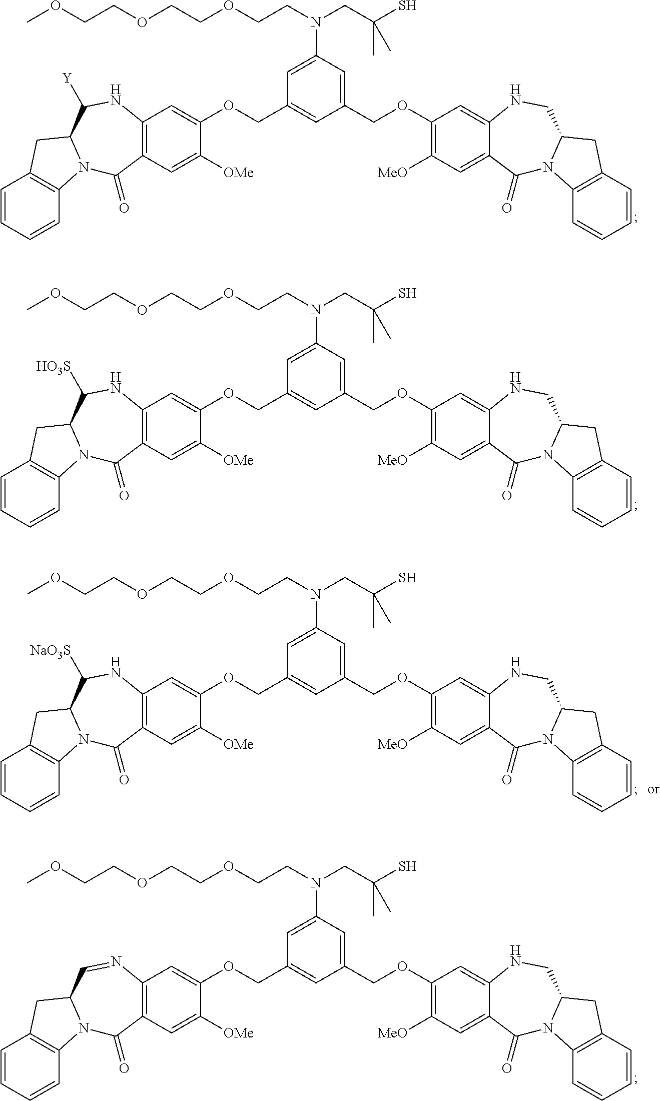
wherein Y is —SO₃M and M is H or a pharmaceutically acceptable cation.

44. The method of claim 43, wherein the tyrosine kinase inhibitor is a FLT3 tyrosine kinase inhibitor.

45. A method for treating or preventing acute myeloid leukemia relapse in a subject, the method comprising administering an effective amount of an immunoconjugate to a pre-selected subject determined to have FLT3-ITD positive acute myeloid leukemia and that has received prior treatment with a tyrosine kinase inhibitor wherein the immunoconjugate comprises a humanized or chimeric antibody or fragment thereof conjugated to a cytotoxic benzodiazepine dimer compound via a cleavable disulfide linker represented by the following structural formula: 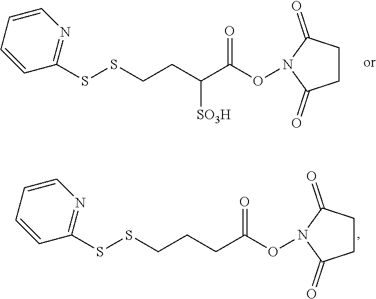 [SMILES: O=C(ON1C(=O)CCC1=O)C(CCSSc1ccccn1)S(=O)(=O)O ; O=C(CCCSSc1ccccn1)ON1C(=O)CCC1=O]
wherein the antibody comprises a heavy chain variable region comprising one or more complementarity determining regions selected from the group consisting of SEQ ID NOs: 1-3; and/or a light chain variable region comprising one or more complementarity determining regions selected from the group consisting of SEQ ID NOs: 4-6; and the cytotoxic benzodiazepine dimer compound represented by one of the following structural formulas or a pharmaceutically acceptable salt thereof: 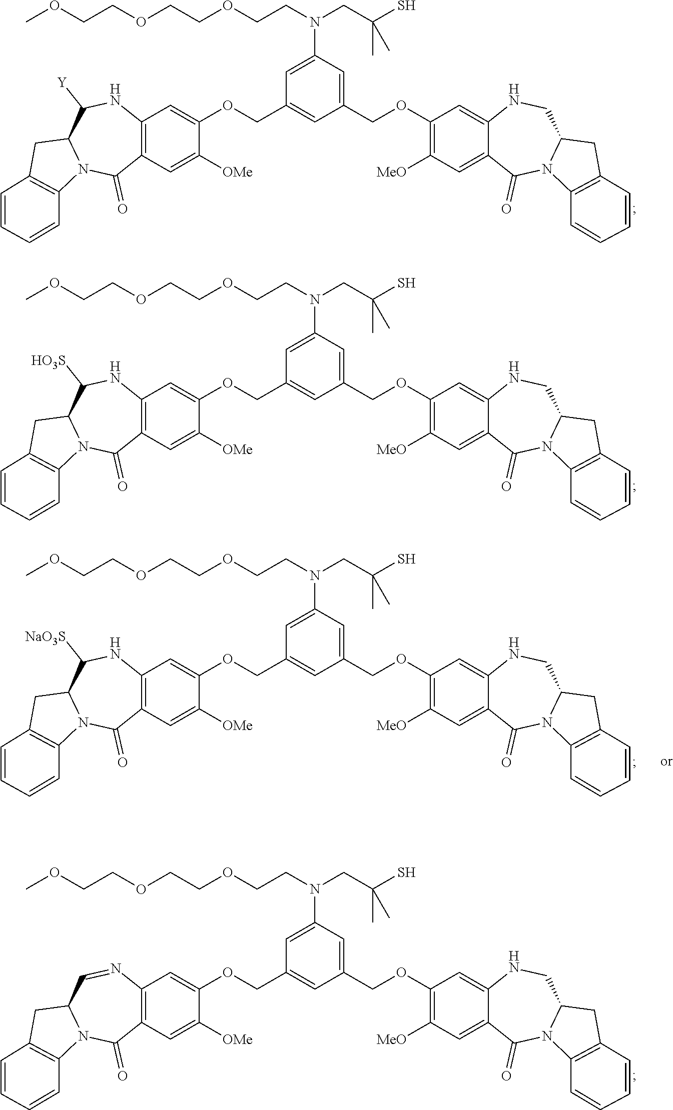
wherein Y is —SO₃M and M is H or a pharmaceutically acceptable cation.

46. The method of claim 45, wherein the tyrosine kinase inhibitor is a FLT3 tyrosine kinase inhibitor.

47. A method for treating a subject having multi-drug resistant acute myeloid leukemia, the method comprising administering an effective amount of an immunoconjugate to a subject, wherein the immunoconjugate comprises a humanized or chimeric antibody or fragment conjugated to a cytotoxic benzodiazepine dimer compound via a cleavable disulfide linker represented by the following structural formula: 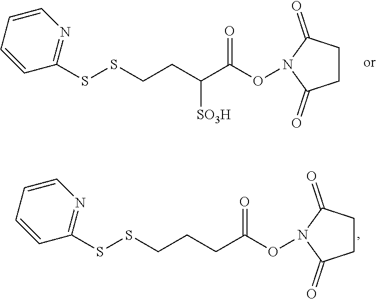 [SMILES: O=C(ON1C(=O)CCC1=O)C(CCSSc1ccccn1)S(=O)(=O)O ; O=C(CCCSSc1ccccn1)ON1C(=O)CCC1=O]
wherein the antibody comprises a heavy chain variable region comprising one or more complementarity determining regions selected from the group consisting of SEQ ID NOs: 1-3; and/or a light chain variable region comprising one or more complementarity determining regions selected from the group consisting of SEQ ID NOs: 4-6; and the cytotoxic benzodiazepine dimer compound represented by one of the following structural formulas or a pharmaceutically acceptable salt thereof: 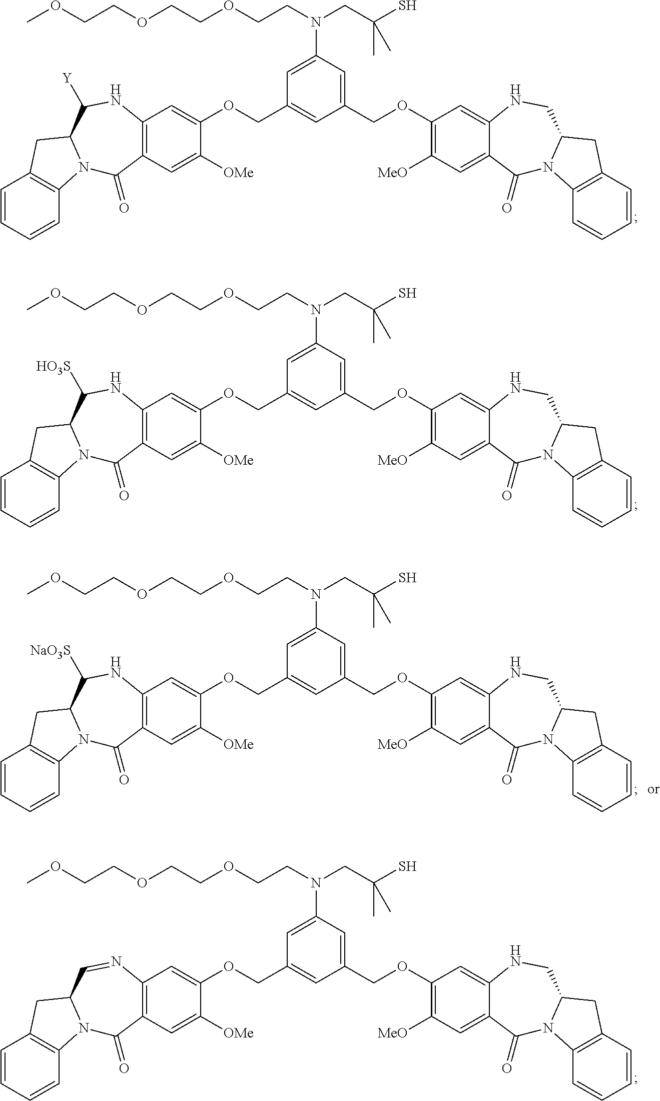
wherein Y is —SO₃M and M is H or a pharmaceutically acceptable cation, thereby treating the multi-drug resistant acute myeloid leukemia.

48. The method of any one of claims 43-47, wherein the subject is identified as having multi-drug resistant leukemia.

49. The method of any one of claims 43-47, wherein the heavy chain variable region comprises an amino acid sequence having at least 95% identity to the amino acid sequence of SEQ ID NO: 7 or 9.

50. The method of any one of claims 43-47, wherein the light chain variable region comprises an amino acid sequence having at least 95% identity to the amino acid sequence of SEQ ID NO: 8 or 10.

51. The method of any one of claims 43-47, wherein the antibody is a humanized My9-6 antibody.

52. The method of claim 51, wherein the humanized antibody is a chimeric or re-surfaced antibody.

53. The method of any one of claims 43-47, wherein the immunoconjugate comprises a humanized My9-6 antibody conjugated to a cytotoxic benzodiazepine dimer compound via N-succinimidyl-4-(2-pyridyldithio)-2-sulfobutanoate, wherein the immunoconjugate is represented by one of the following structural formulas or a pharmaceutically acceptable salt thereof: 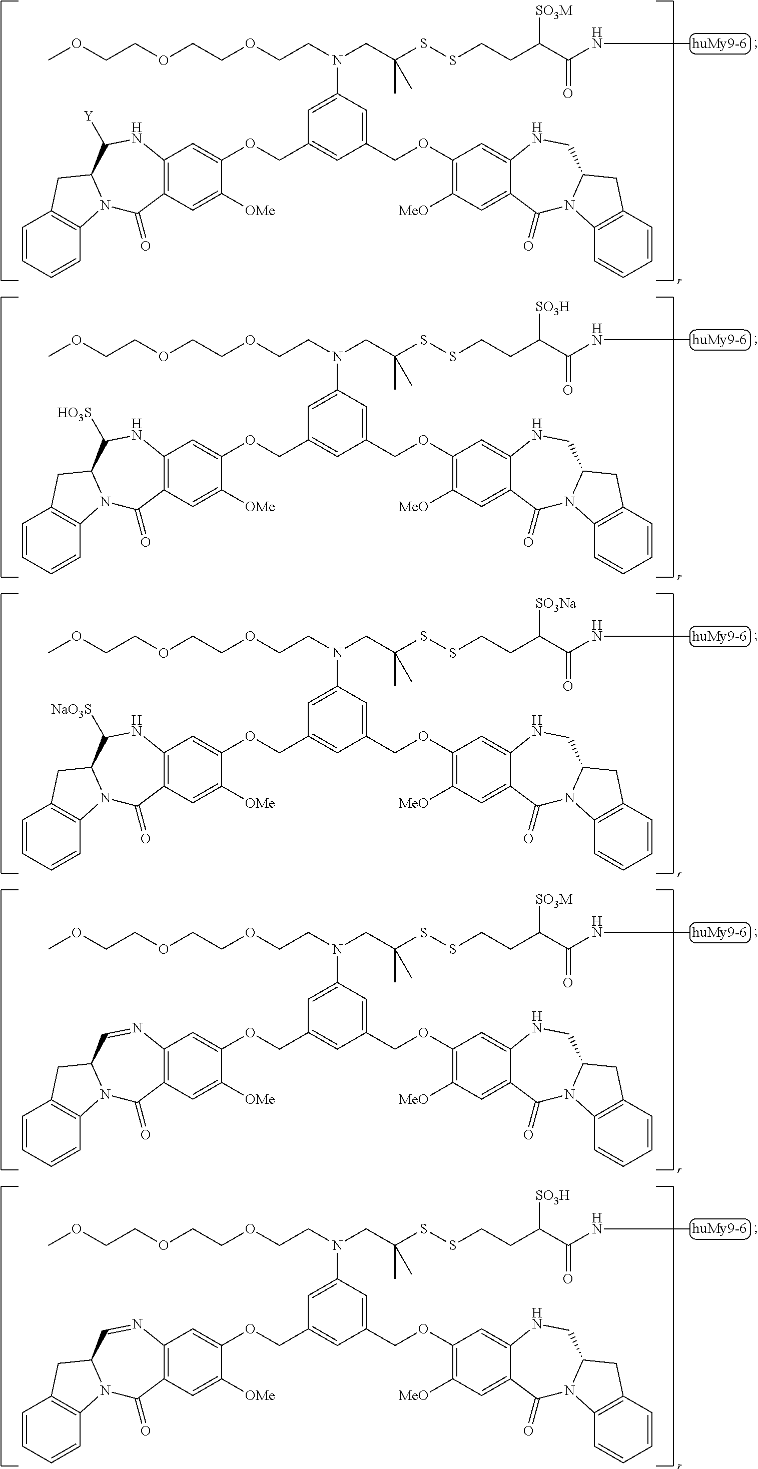 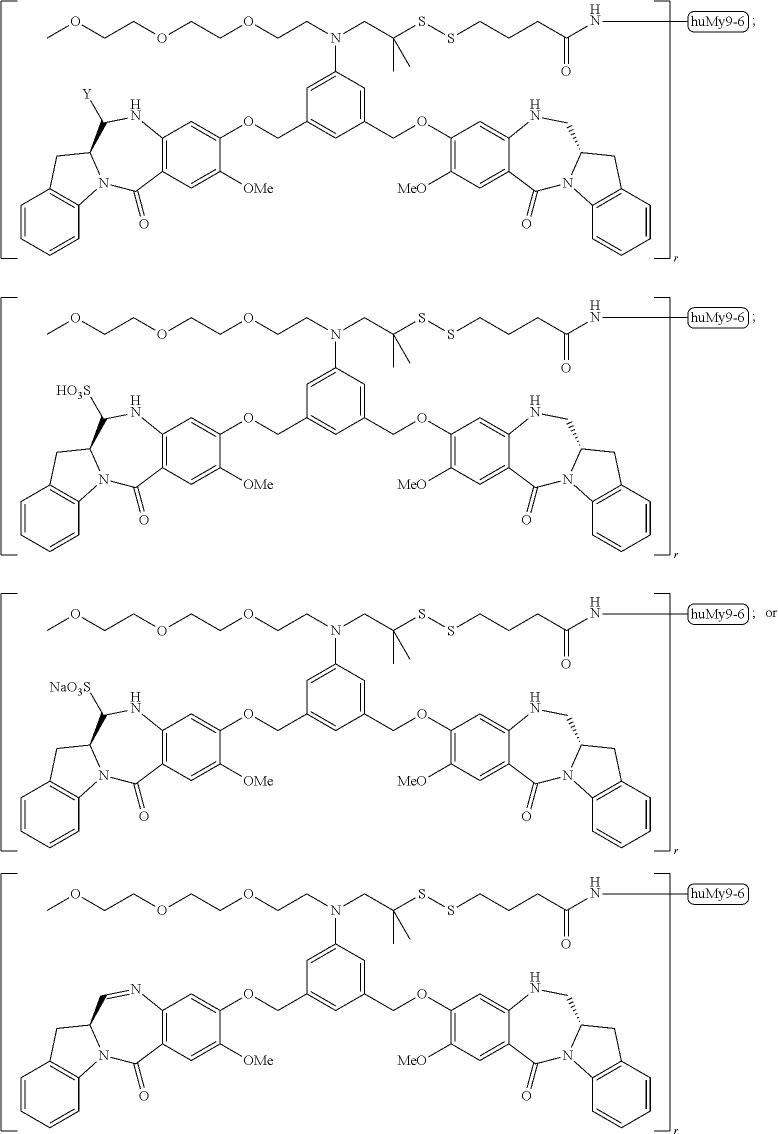
wherein r is an integer from 1 to 10, Y is —SO₃M and M, for each occurrence, is independently —H or a pharmaceutically acceptable cation;

54. The method of any one of claims 43-47, wherein the subject is identified as having multi-drug resistant leukemia by detecting the presence of P-glycoprotein expression in a peripheral blood or bone marrow sample of the subject.

55. The method of claim 54, further comprising detecting the presence of CD33 expression in a peripheral blood or bone marrow sample of the subject.

56. The method of claim 55, wherein a level greater than about 1,000, 3,000, or 5,000 CD33 antigens per cell identifies the AML as responsive to treatment with the immunoconjugate.

57. A method for treating or preventing acute myeloid leukemia relapse in a subject, comprising administering an effective amount of an immunoconjugate to the subject, wherein the immunoconjugate comprises a humanized or chimeric antibody or fragment conjugated to a cytotoxic benzodiazepine dimer compound via a cleavable disulfide linker represented by the following structural formula: 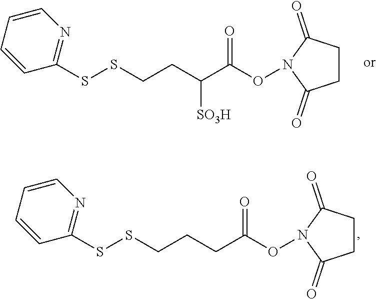 [SMILES: O=C(ON1C(=O)CCC1=O)C(CCSSc1ccccn1)S(=O)(=O)O ; O=C(CCCSSc1ccccn1)ON1C(=O)CCC1=O]
wherein the antibody comprises a heavy chain variable region comprising one or more complementarity determining regions selected from the group consisting of SEQ ID NOs: 1-3; and/or a light chain variable region comprising one or more complementarity determining regions selected from the group consisting of SEQ ID NOs: 4-6; and the cytotoxic benzodiazepine dimer compound represented by one of the following structural formulas or a pharmaceutically acceptable salt thereof: 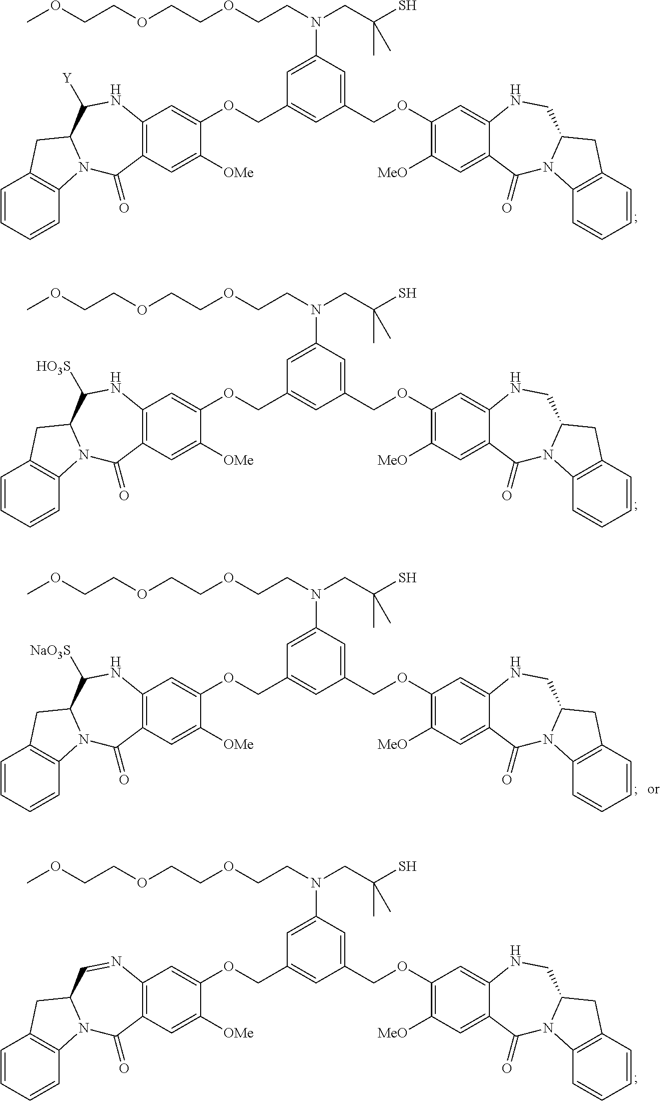
wherein Y is —SO₃M and M is H or a pharmaceutically acceptable cation, thereby treating the acute myeloid leukemia relapse.

58. The method of claim 57, wherein the heavy chain variable region comprises an amino acid sequence having at least 95% identity to the amino acid sequence of SEQ ID NO:7 or 9.

59. The method of claim 57, wherein the light chain variable region comprises an amino acid sequence having at least 95% identity to the amino acid sequence of SEQ ID NO: 8 or 10.

60. The method of claim 57, wherein the antibody is a humanized My9-6 antibody.

61. The method of claim 57, wherein the humanized antibody is a re-surfaced or CDR-grafted antibody.

62. The method of claim 57, wherein the immunoconjugate comprises a humanized My9-6 antibody conjugated to a cytotoxic benzodiazepine dimer compound via N-succinimidyl-4-(2-pyridyldithio)-2-sulfobutanoate, wherein the immunoconjugate is represented by one of the following structural formulas or a pharmaceutically acceptable salt thereof: 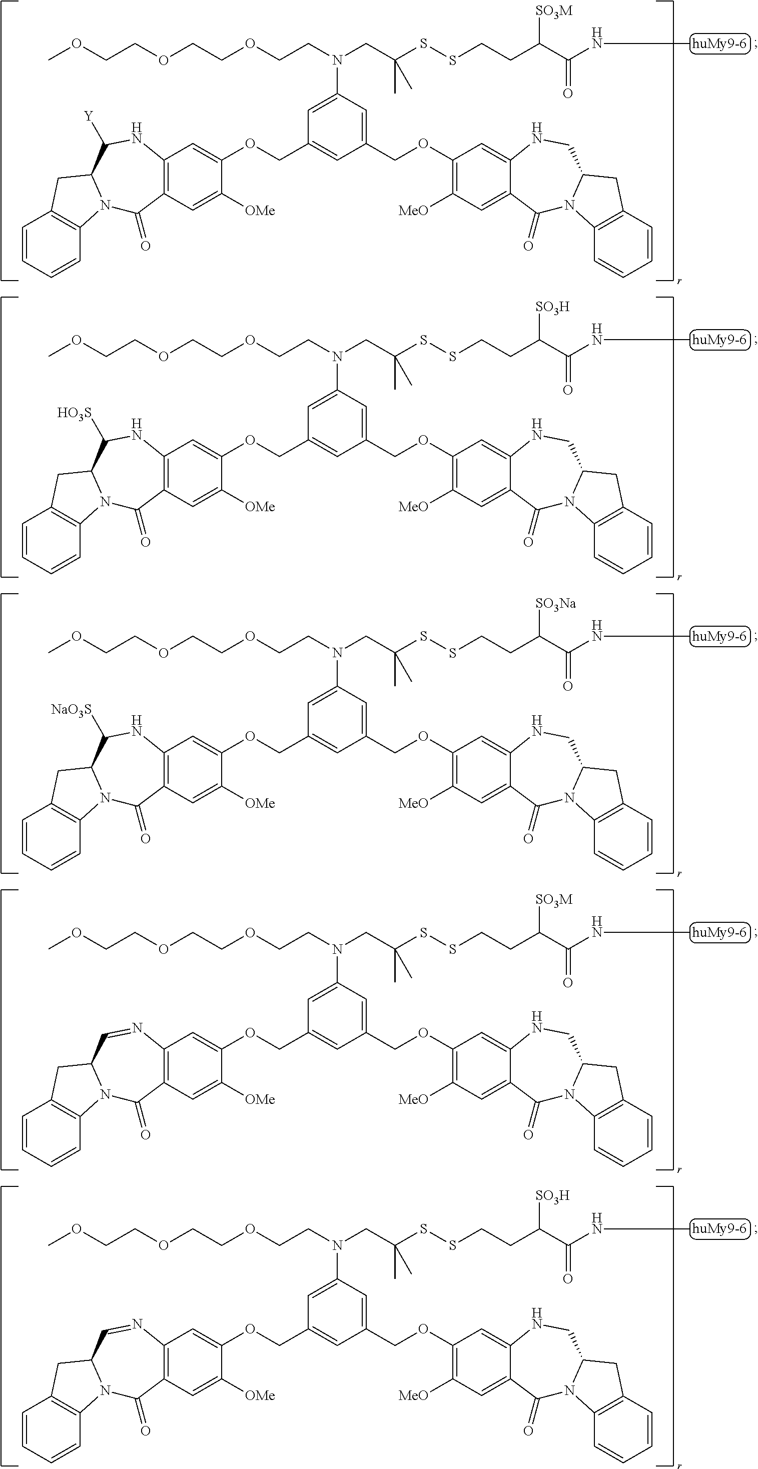 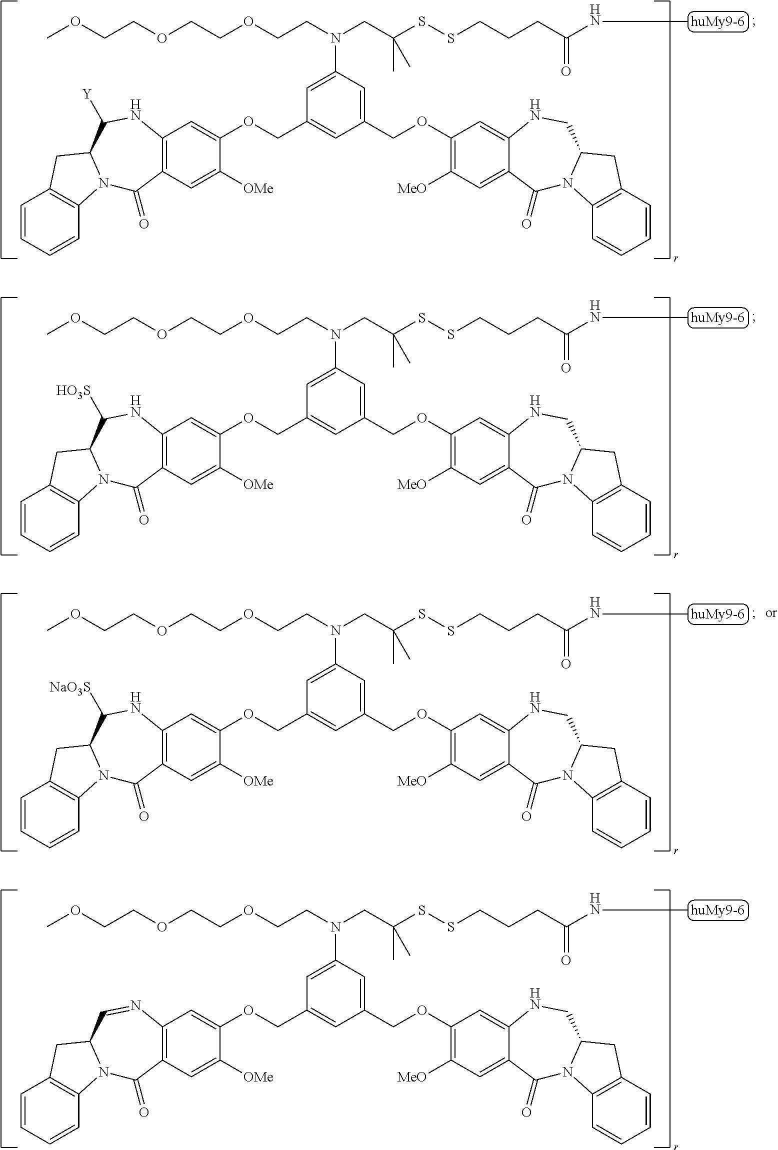
wherein r is an integer from 1 to 10, Y is —SO₃M and M, for each occurrence, is independently —H or a pharmaceutically acceptable cation.

63. The method of claim 54, wherein the method prevents, reduces, or eliminates minimal residual disease.

64. The method of claim 54, wherein the antibody specifically binds a CD33-expressing leukemic progenitor and/or leukemic stem cell.

65. The method of claim 54, wherein the method spares normal hematopoietic stem cells.

66. A method for inducing cell death in a leukemic stem cell, the method comprising contacting the leukemic stem cell with an effective amount of an immunoconjugate comprising a humanized or chimeric antibody or fragment conjugated to a cytotoxic benzodiazepine dimer compound via a cleavable disulfide linker represented by the following structural formula: 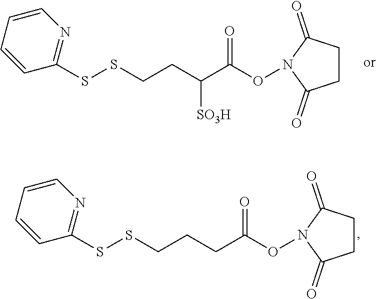 [SMILES: O=C(ON1C(=O)CCC1=O)C(CCSSc1ccccn1)S(=O)(=O)O ; O=C(CCCSSc1ccccn1)ON1C(=O)CCC1=O]
wherein the antibody comprises a heavy chain variable region comprising one or more complementarity determining regions selected from the group consisting of SEQ ID NOs: 1-3; and/or a light chain variable region comprising one or more complementarity determining regions selected from the group consisting of SEQ ID NOs: 4-6; and the cytotoxic benzodiazepine dimer compound represented by one of the following structural formulas or a pharmaceutically acceptable salt thereof: 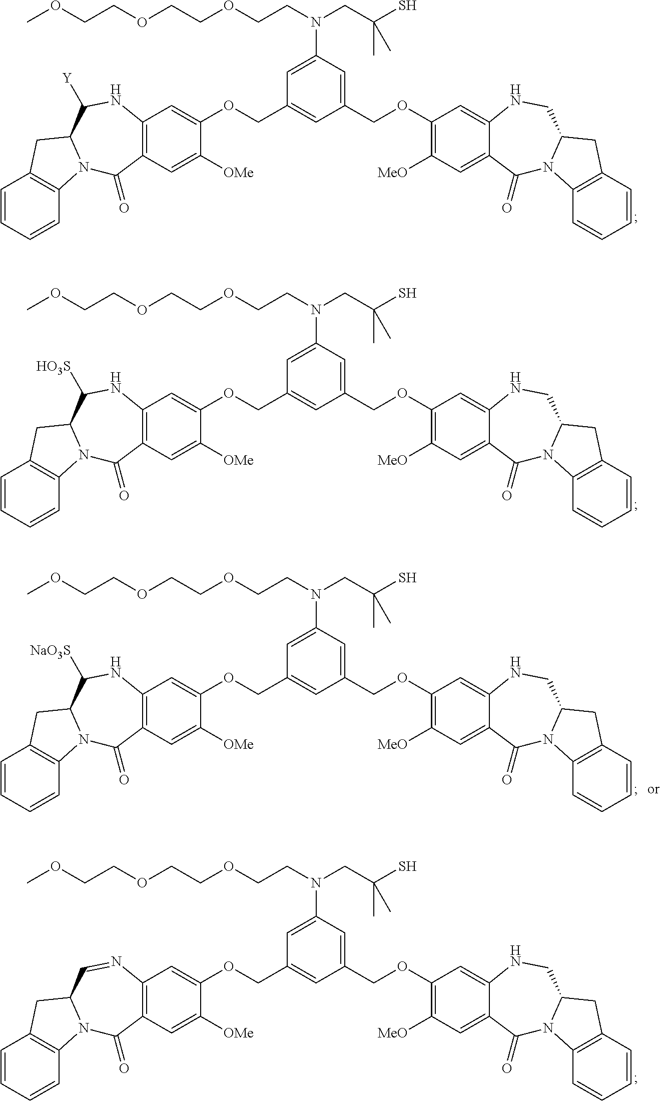
wherein Y is —SO₃M and M is H or a pharmaceutically acceptable cation, thereby inducing cell death in the leukemic stem cell.

67. A method for inducing cell death in a FLT3-ITD positive leukemic cell, the method comprising contacting the leukemic stem cell with an effective amount of an immunoconjugate comprising a humanized or chimeric antibody or fragment conjugated to a cytotoxic benzodiazepine dimer compound via a cleavable disulfide linker represented by the following structural formula: 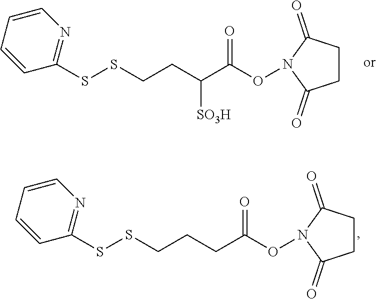 [SMILES: O=C(ON1C(=O)CCC1=O)C(CCSSc1ccccn1)S(=O)(=O)O ; O=C(CCCSSc1ccccn1)ON1C(=O)CCC1=O]
wherein the antibody comprises a heavy chain variable region comprising one or more complementarity determining regions selected from the group consisting of SEQ ID NOs: 1-3; and/or a light chain variable region comprising one or more complementarity determining regions selected from the group consisting of SEQ ID NOs: 4-6; and the cytotoxic benzodiazepine dimer compound represented by one of the following structural formulas or a pharmaceutically acceptable salt thereof: 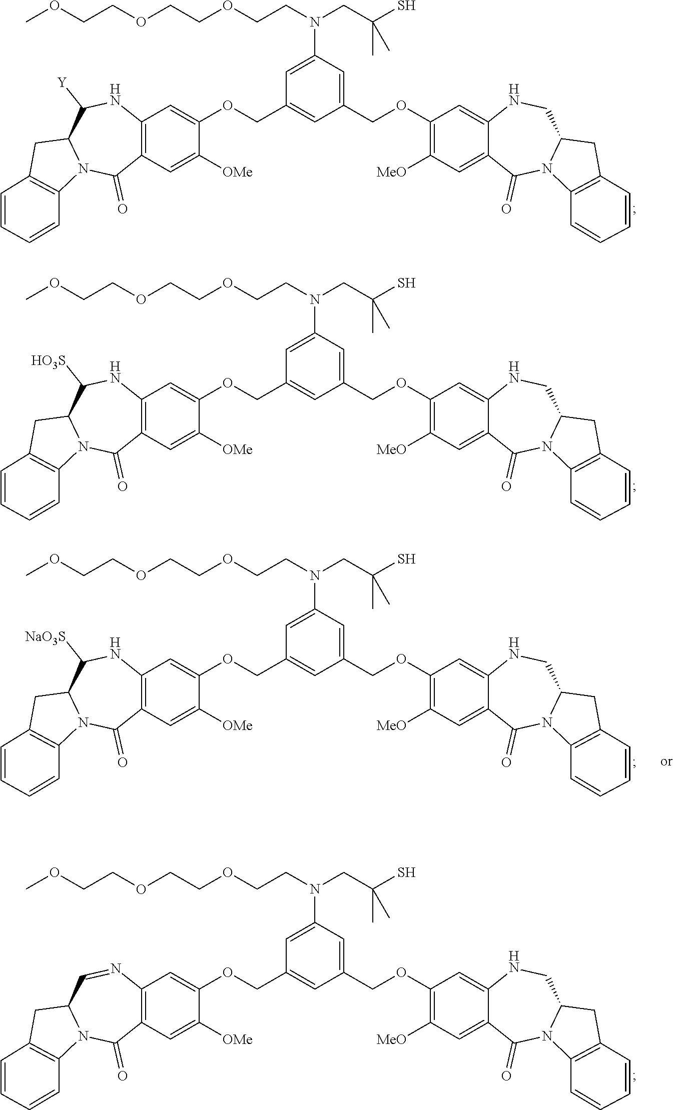
wherein Y is —SO₃M and M is H or a pharmaceutically acceptable cation, thereby inducing cell death in the FLT3-ITD positive leukemic cell.

68. The method of claim 66 or 67, wherein the heavy chain variable region comprises an amino acid sequence having at least 95% identity to the amino acid sequence of SEQ ID NO:7 or 9.

69. The method of claim 66 or 67, wherein the light chain variable region comprises an amino acid sequence having at least 95% identity to the amino acid sequence of SEQ ID NO: 8 or 10.

70. The method of claim 66 or 67, wherein the antibody is a humanized My9-6 antibody.

71. The method of claim 70, wherein the humanized antibody is a re-surfaced or CDR-grafted antibody.

72. The method of claim 66 or 67, wherein the immunoconjugate comprises a humanized My9-6 antibody conjugated to a cytotoxic benzodiazepine dimer compound via N-succinimidyl-4-(2-pyridyldithio)-2-sulfobutanoate, wherein the immunoconjugate is represented by one of the following structural formulas or a pharmaceutically acceptable salt thereof: 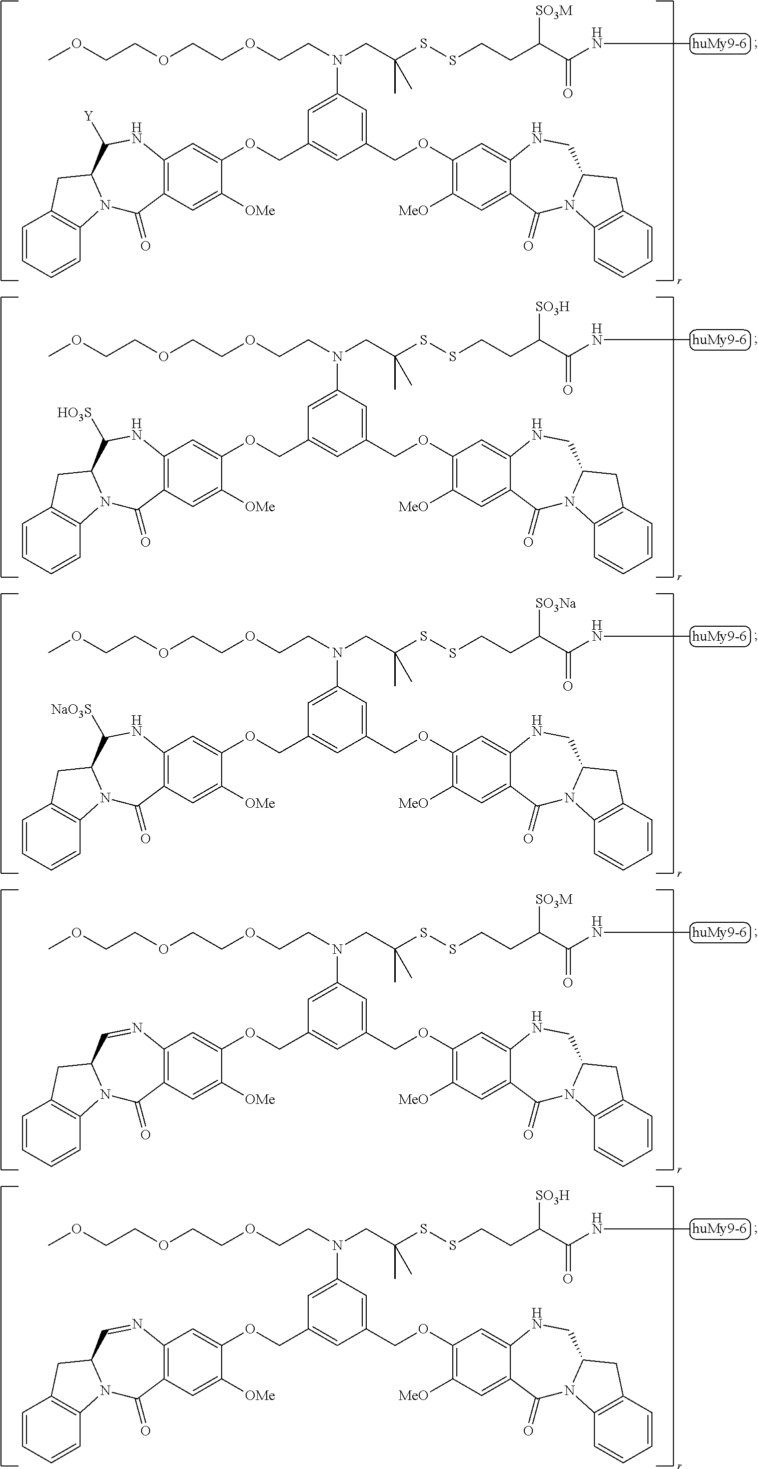 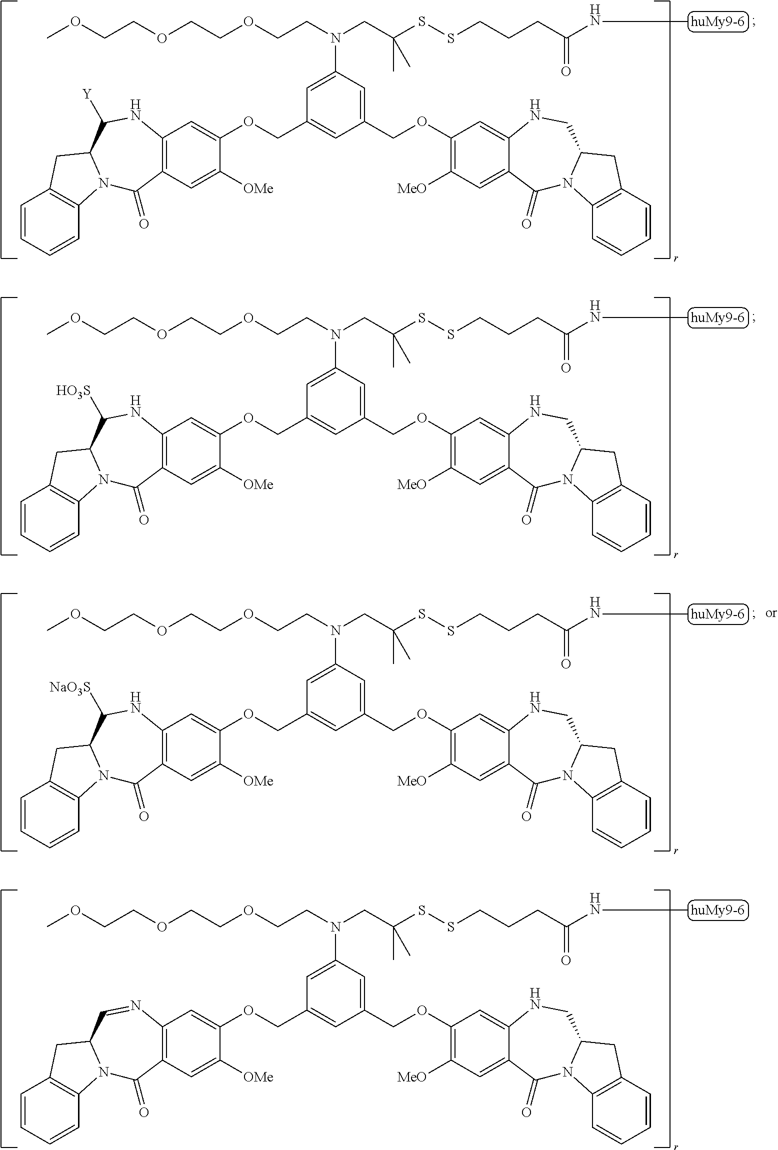
wherein r is an integer from 1 to 10, Y is —SO₃M and M, for each occurrence, is independently —H or a pharmaceutically acceptable cation.

73. The method of claim 66 or 67, wherein the method does not induce cell death in a normal hematopoietic stem cell.

74. The method of claim 66 or 67, wherein the contacting is in vitro or in vivo.

75. The method of claim 66 or 67, wherein the leukemic stem cell is in a subject newly diagnosed with acute myeloid leukemia, in a subject identified as having a relapse associated with the growth or proliferation of a leukemic stem cell, or in a subject identified as having refractory acute myeloid leukemia.

76. The method of any one of claims 1-75, wherein the immunoconjugate has an IC₅₀ value from about 10 pM to about 2 nM.

77. The method of claim 76, wherein the immunoconjugate has an IC₅₀ value from about 11 pM to about 1.6 nM.

78. The method of any one of claims 1-77, wherein the method preferentially kills leukemic stem cells.

79. The method of any one of claims 1-78, wherein the antibody comprises at least one heavy chain variable region or fragment thereof and at least one light chain variable region or fragment thereof, wherein said at least one heavy chain variable region or fragment thereof comprises three sequential complementarity-determining regions having amino acid sequences set forth in SEQ ID NOs:1-3, respectively, and wherein said at least one light chain variable region or fragment thereof comprises three sequential complementarity-determining regions having amino acid sequences set forth in SEQ ID NOs:4-6, respectively.

80. The method of any one of claims 1-79, wherein the antibody or fragment thereof comprises: a heavy chain variable region CDR1 having the amino acid sequence of SEQ ID NO:1; a heavy chain variable region CDR2 having the amino acid sequence of SEQ ID NO:2; a heavy chain variable region CDR3 having the amino acid sequence of SEQ ID NO:3; a light chain variable region CDR1 having the amino acid sequence of SEQ ID NO:4; a light chain variable region CDR2 having the amino acid sequence of SEQ ID NO:5; and a light chain variable region CDR3 having the amino acid sequence of SEQ ID NO:6.

81. A kit comprising an anti-CD33 antibody and a therapeutic composition comprising an effective amount of an immunoconjugate comprising a humanized My9-6 antibody linked by N-succinimidyl-4-(2-pyridyldithio)-2-sulfobutanoate to a cytotoxic benzodiazepine dimer compound, wherein the immunoconjugate is represented by one of the following structural formulas or a pharmaceutically acceptable salt thereof: 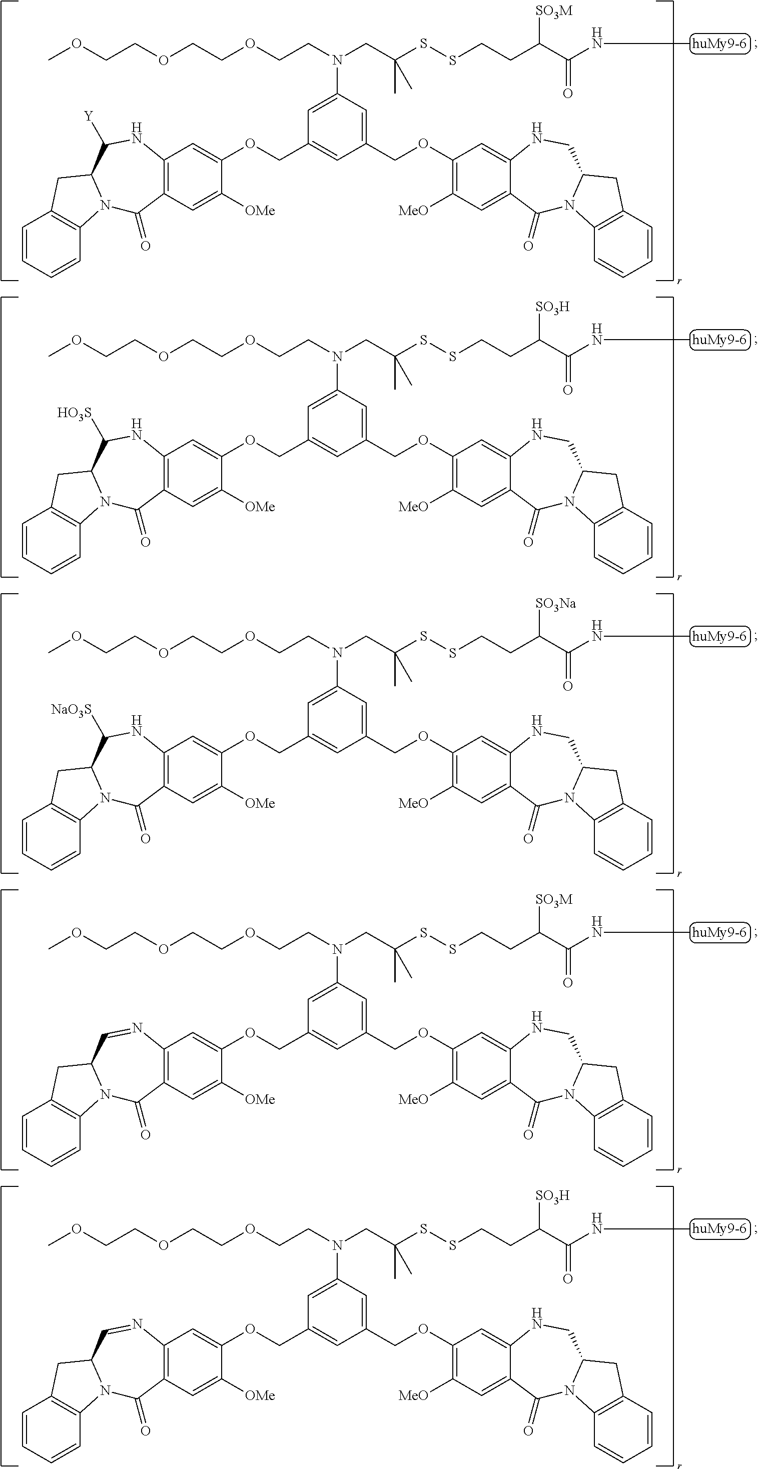 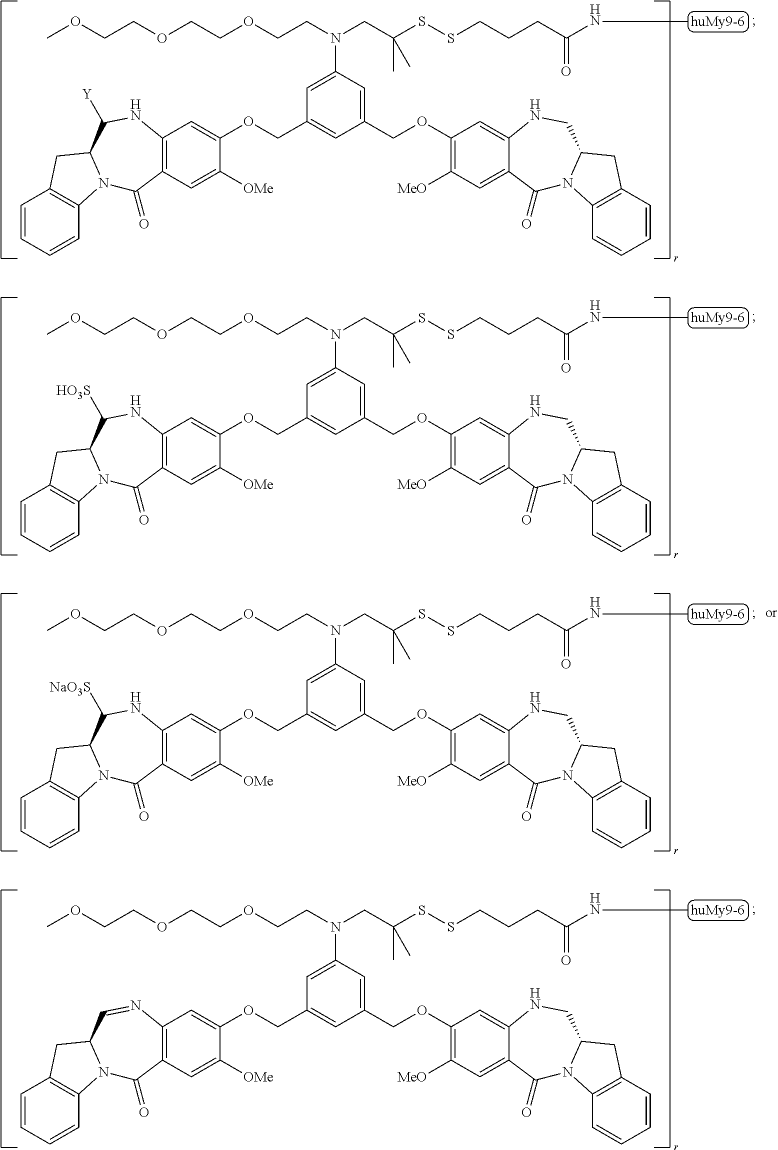
wherein r is an integer from 1 to 10, Y is —SO₃M and M, for each occurrence, is independently —H or a pharmaceutically acceptable cation.

82. The kit of claim 81, wherein the kit further comprises directions for detecting the level of CD33 expression in a sample from a subject using the anti-CD33 antibody.

83. The kit of claim 81, further comprising instructions for administering the immunoconjugate to a subject identified as having at least about 1,000 antigens per cell.

84. The kit of claim 83, wherein the subject is identified as having at least about 3,000 or 5,000 antigens per cell.
